(19) United States
(12) Patent Application Publication
(10) Pub. No.: US 2024/0101630 A1
(43) Pub. Date: Mar. 28, 2024

(54) BI-FUNCTIONAL LINEAR FUSION COLLAGEN-LOCALIZED IMMUNOMODULATORY MOLECULES AND METHODS THEREOF

(71) Applicant: CULLINAN AMBER CORP., Cambridge, MA (US)

(72) Inventors: Naveen Mehta, Cambridge, MA (US); Jennifer Michaelson, Cambridge, MA (US); Patrick Baeuerle, Cambridge, MA (US); Li Bochong, Cambridge, MA (US); Dane K. Wittrup, Cambridge, MA (US)

(21) Appl. No.: 18/257,177

(22) PCT Filed: Dec. 17, 2021
(86) PCT No.: PCT/US2021/064224

§ 371 Date: Jun. 13, 2023

Related U.S. Application Data
(60) Provisional application No. 63/127,995, filed on Dec. 18, 2020.

(51) Int. Cl. C07K 14/55; C07K 14/54

(52) U.S. Cl. CPC C07K 14/55; C07K 14/5434; A61K 38/00; C07K 2319/31

(57) ABSTRACT

Disclosed herein are immunomodulatory fusion proteins comprising an IL-2; an IL-12, a collagen-binding domain, and a linear polypeptide spacer, methods of making and using the same. The immunomodulatory fusion proteins disclosed herein are useful for the treatment of cancer.

### CROSS-REFERENCE TO RELATED APPLICATION

The present application claims the benefit of and priority to U.S. Provisional Patent Application No. 63/127,995, filed Dec. 18, 2020, the disclosures of which are hereby incorporated by reference in their entireties for all purposes.

### BACKGROUND

While immunotherapy has transformed oncology with durable curative responses in a minority of patients, immune-related adverse events (irAEs) limit its broadest application (Michot et al. 2016, Eur J Cancer, 54: 139-148). It is desired to restrict the most potent immune activation events to tumor tissue, while sparing non-tumor healthy tissue. Various tumor-localization approaches have been proposed: linking immunomodulatory agents to tumor-targeting modules in immunocytokines (Hutmacher and Neri 2018, Adv Drug Deliv Rev); masking agent activity systemically, with tumor-localized proteolytic activation (Thomas and Daugherty 2009, Protein Sci 18:2053-2059); intratumoral injection of the agents (Singh and Overwijk 2015, Nat Commun 8:1447; Ager et al. 2017, Cancer Immunol Res 5:676-684; Bommareddy et al. 2017, Cancer J 23:40-47; Milling et al. 2017, Adv Drug Deliv Rev 114:79-101; Singh et al. 2017, Nat Commun 8:1447; Sagiv-Barfi et al. 2018, Sci Transl Med 10:eaan4488); peritumoral injection of a solid biomaterial to entrap the agent (Park et al. 2018, Sci Transl Med, 10:eaar1916); conjugation to a solid particle (Kwong et al. 2013, Cancer Res 73:1547-1558) or conjugation of basic charged peptides to drive some nonspecific sticking of the agent to tumor extracellular matrix (Ishihara et al. 2017, Sci Transl Med 9:eaan0401; Ishihara et al. 2018, Mal Cancer Ther 17:2399-2411). A related but distinct approach is to localize growth factors in tissue to drive tissue regeneration (Nishi et al. 1998, Proc Natl Acad Sci 95:7018-7023; Martino et al. 2014, Science 343:885-888; Mitchell et al. 2016, Acta Biomater 30:1-12).

Significant problems exist with each of the current approaches above. Immunocytokines systemically expose immune cells to the immunomodulatory agent (Tzeng et al. 2015, Proc Natl Acad Sci 112:3320-3325). Masking agents may be unmasked outside target tissues, and the masking agent may complicate manufacturing and immunogenicity. Intratumoral injection often leads to rapid diffusion out of the tumor compartment. Conjugation of peptides at random sites is difficult to reproduce, can negatively impact specific activity, doesn't fully prevent tumor exit, and creates significant CMC issues due to the heterogeneous products of random conjugation methods.

Accordingly, there remains a need for novel immunotherapy approaches to promote tumor-localization and increase efficacy, while preventing systemic toxicity.

### SUMMARY

Described herein are compounds, compositions, and methods for treating cancer. The compounds include a fusion protein including each of an IL-2, an IL-12, a collagen-binding domain, and a linear polypeptide spacer. When administered to a subject, the compounds have a favorable residence time in the tumor and can provide in some embodiments treatments with acceptable toxicity an enhanced therapeutic index. In some embodiments, the collagen-binding domain binds to the collagen in the tumor to maintain localization of the compound in the tumor for an extended period of time.

Disclosed herein is an immunomodulatory fusion protein comprising: (i) an IL-2; (ii) an IL-12; (iii) a collagen-binding domain, and (iv) a linear polypeptide spacer.

In various embodiments, the immunomodulatory fusion protein is linear. In various embodiments, the immunomodulatory fusion protein is a continuous chain. In various embodiments, the immunomodulatory fusion protein is a continuous polypeptide chain.

In various embodiments, the IL-2 is at the N-terminus of the immunomodulatory fusion protein. In various embodiments, the IL-12 is at the C-terminus of the immunomodulatory fusion protein. In various embodiments, the IL-2 is at the N-terminus of the immunomodulatory fusion protein and the IL-12 is at the C-terminus of the immunomodulatory fusion protein.

In various embodiments, the linear polypeptide spacer is positioned in between the IL-2 and the collagen-binding domain. In various embodiments, the collagen-binding domain is positioned in between the IL-12 and the linear polypeptide spacer.

In various embodiments, the C-terminus of the IL-2 is operably linked to the N-terminus of the linear polypeptide spacer. In various embodiments, the C-terminus of the IL-2 is operably linked by a linker to the N-terminus of the linear polypeptide spacer.

In various embodiments, the C-terminus of the linear polypeptide spacer is operably linked to the N-terminus of the collagen-binding domain. In various embodiments, the C-terminus of the linear polypeptide spacer is operably linked by a linker to the N-terminus of the collagen-binding domain.

In various embodiments, the C-terminus of the collagen-binding domain is operably linked to the N-terminus of the IL-12. In various embodiments, the C-terminus of the collagen-binding domain is operably linked by a linker to the N-terminus of the IL-12.

In various embodiments, the collagen-binding domain is positioned in between the IL-2 and the linear polypeptide spacer. In various embodiments, the linear polypeptide spacer is positioned in between the IL-12 and the collagen-binding domain. In various embodiments, the C-terminus of the IL-2 is operably linked to the N-terminus of the collagen-binding domain.

In various embodiments, the C-terminus of the IL-2 is operably linked by a linker to the N-terminus of the collagen-binding domain. In various embodiments, the C-terminus of the collagen-binding domain is operably linked to the N-terminus of the linear polypeptide spacer.

In various embodiments, the C-terminus of the collagen-binding domain is operably linked by a linker to the N-terminus of the linear polypeptide spacer. In various embodiments, the C-terminus of the linear polypeptide spacer is operably linked to the N-terminus of the IL-12. In various embodiments, the C-terminus of the linear polypeptide spacer is operably linked by a linker to the N-terminus of the IL-12.

In various embodiments, the IL-2 is at the C-terminus of the immunomodulatory fusion protein. In various embodiments, the IL-12 is at the N-terminus of the immunomodulatory fusion protein. In various embodiments, the IL-2 is at the C-terminus and the IL-12 is at the N-terminus of the immunomodulatory fusion protein.

In various embodiments, the N-terminus of the IL-2 is operably linked to the C-terminus of the linear polypeptide spacer. In various embodiments, the N-terminus of the IL-2 is operably linked by a linker to the C-terminus of the linear polypeptide spacer.

In various embodiments, the N-terminus of the linear polypeptide spacer is operably linked to the C-terminus of the collagen-binding domain. In various embodiments, the N-terminus of the linear polypeptide spacer is operably linked by a linker to the C-terminus of the collagen-binding domain.

In various embodiments, the N-terminus of the collagen-binding domain is operably linked to the C-terminus of the IL-12. In various embodiments, the N-terminus of the collagen-binding domain is operably linked by a linker to the C-terminus of the IL-12.

In various embodiments, the collagen-binding domain is positioned in between the IL-2 and the linear polypeptide spacer. In various embodiments, the linear polypeptide spacer is positioned in between the IL-12 and the collagen-binding domain.

In various embodiments, the N-terminus of the IL-2 is operably linked to the C-terminus of the collagen-binding domain. In various embodiments, the N-terminus of the IL-2 is operably linked by a linker to the C-terminus of the collagen-binding domain.

In various embodiments, the N-terminus of the collagen-binding domain is operably linked to the C-terminus of the linear polypeptide spacer. In various embodiments, the N-terminus of the collagen-binding domain is operably linked by a linker to the C-terminus of the linear polypeptide spacer.

In various embodiments, the N-terminus of the linear polypeptide spacer is operably linked to the C-terminus of the IL-12. In various embodiments, the N-terminus of the linear polypeptide spacer is operably linked by a linker to the C-terminus of the IL-12.

In various embodiments, one or more of the linkers are the same. In various embodiments, one or more of the linkers are the different.

In various embodiments, the IL-12 is at the C terminus of the immodulatory fusion protein and is operably linked to the collagen binding domain, which is operably linked to a linear polypeptide spacer, which is operably linked to the IL-2 at the N terminus of the protein, and wherein the protein is linear.

In various embodiments, the IL-12 is at the N terminus of the immodulatory fusion protein and is operably linked to the collagen binding domain, which is operably linked to a linear polypeptide spacer, which is operably linked to the IL-2 at the C terminus of the protein, and wherein the protein is linear.

In various embodiments, the IL-12 is at the C terminus of the immodulatory fusion protein and is operably linked to the linear polypeptide spacer, which is operably linked to collagen binding domain, which is operably linked to the IL-2 at the N terminus of the protein, and wherein the protein is linear.

In various embodiments, the IL-12 is at the N terminus of the immodulatory fusion protein and is operably linked to the linear polypeptide spacer, which is operably linked to collagen binding domain, which is operably linked to the IL-2 at the C terminus of the protein, and wherein the protein is linear.

In various embodiments, the immodulatory fusion protein further comprises a second linear polypeptide spacer.

In various embodiments, the IL-12 is at the N terminus of the immodulatory fusion protein and is operably linked to the first linear polypeptide spacer, which is operably linked to the collagen binding domain, which is operably linked to the second linear polypeptide spacer, which is operably linked to the IL-2 at the C terminus of the protein, and wherein the protein is linear.

In various embodiments, the IL-12 is at the C terminus of the immodulatory fusion protein and is operably linked to the first linear polypeptide spacer, which is operably linked to the collagen binding domain, which is operably linked to the second linear polypeptide spacer, which is operably linked to the IL-2 at the N terminus of the protein, and wherein the protein is linear.

In various embodiments, the immodulatory fusion protein is a continuous chain. In various embodiments, the immodulatory fusion protein is a continuous polypeptide chain.

In various embodiments, the collagen-binding domain comprises (i) a leucine-rich repeat from a human proteoglycan Class II member of the small leucine-rich proteoglycan (SLRP) family which comprises lumican; or (ii) a human type I glycoprotein having an Ig-like domain selected from LAIR1 and LAIR2.

In various embodiments, the collagen-binding domain comprises lumican. In various embodiments, the lumican comprises at least about 80% sequence identity to the amino acid sequence as set forth in SEQ ID NO: 11.

In various embodiments, the collagen-binding domain comprises LAIR 1. In various embodiments, the LAIR1 comprises at least about 80% sequence identity to the amino acid sequence as set forth in SEQ ID NO: 13. In various embodiments, the LAIR1 comprises at least 80% identity to the amino acid as set forth in SEQ ID NO: 14.

In various embodiments, the collagen-binding domain comprises LAIR 2. In various embodiments, the LAIR2 comprises at least 80% identity to the amino acid sequence as set forth in SEQ ID NO: 15.

In various embodiments, the IL-2 comprises human IL-2. In various embodiments, the IL-2 comprises human wild-type IL-2. In various embodiments, the IL-2 comprises at least about 80% sequence identity to the amino acid sequence as set forth in SEQ ID NO: 1. In various embodiments, the IL-2 comprises at least about 80% sequence identity to the amino acid sequence as set forth in SEQ ID NO: 2.

In various embodiments, the IL-12 comprises human IL-12. In various embodiments, the IL-12 comprises human wild-type IL-12. In various embodiments, the IL-12 comprises at least about 80% sequence identity to the amino acid sequence as set forth in SEQ ID NO: 5. In various embodiments, the IL-12 comprises at least about 80% sequence identity to the amino acid sequence as set forth in SEQ ID NO: 6.

In various embodiments, the linear polypeptide spacer is an albumin. In various embodiments, the linear polypeptide spacer is an albumin binding domain. In various embodiments, the albumin comprises human albumin.

In various embodiments, the albumin comprises at least about 80% sequence identity to the amino acid sequence as set forth in SEQ ID NOs: 16-18. In various embodiments, the albumin binding domain comprises at least about 80% sequence identity to the amino acid sequence as set forth in SEQ ID NO: 19.

In various embodiments, the immunomodulatory fusion protein molecular weight is at least about 100-1000 kDa.

Additionally disclosed herein is an pharmaceutical composition comprising an immunomodulatory fusion protein of any one of the immunomodulatory fusion proteins disclosed herein, and a pharmaceutically acceptable carrier.

Additionally disclosed herein is a method for activating, enhancing or promoting a response by an immune cell in a subject or inhibiting, reducing or suppressing a response by an immune cell in a subject, comprising administering to a subject in need thereof, an effective amount of the pharmaceutical composition of any one of the pharmaceutical composition disclosed herein.

Additionally disclosed herein is a method for treating cancer, or reducing or inhibiting tumor growth, comprising administering to a subject in need thereof, an effective amount of the pharmaceutical composition of any one of the pharmaceutical composition disclosed herein.

In various embodiments, the subject has at least one tumor. In various embodiments, the composition is administered intratumorally (i.tu) or peritumorally (peri.tu) to the at least one tumor. In various embodiments, the at least one tumor size is reduced or substantially identical to a reference standard. In various embodiments, the reference standard is the size of the tumor prior to administration.

In various embodiments, the composition is administered by injection.

In various embodiments, the composition has an intratumoral retention ti/2 of more than 24 hours.

In various embodiments, twelve hours after intratumoral injection less then 25% of the injected dose is detected in the serum.

In various embodiments, the at least one tumor has stromal CD8+ cytotoxic T cells (CTL)≤50 cells/mm2. In various embodiments, the at least one tumor has stromal CD8+ cytotoxic T cells (CTL)≥50 cells/mm2 and intraepithelial compartment CD8+ cytotoxic T cells (CTL)≤500 cells/mm2. In various embodiments, the at least one tumor has intraepithelial compartment CD8+ cytotoxic T cells (CTL)≥500 cells/mm2.

In various embodiments, the method does not result in cytokine release syndrome in the subject. In various embodiments, the subject does not experience grade 4 cytokine release syndrome.

Additionally disclosed herein is a method for reducing or inhibiting tumor growth or treating cancer in a subject, the method comprising administering to a subject in need thereof, an effective amount of the pharmaceutical composition of any one of the pharmaceutical composition disclosed herein, and an effective amount of a second composition comprising (i) a tumor antigen-targeting antibody, (ii) a cancer vaccine, (iii) an immune checkpoint inhibitor, or (iv) an adoptive cell therapy, thereby reducing or inhibiting tumor growth or treating cancer in the subject.

In various embodiments, the tumor antigen is a tumor-associated antigen (TAA), a tumor specific antigen (TSA), or a tumor neoantigen and/or wherein the tumor antigen-targeting antibody specifically binds human HER-2/neu, EGFR, VEGFR, CD20, CD33, CD38 or antigen-binding fragment thereof. In various embodiments, the cancer vaccine is a peptide comprising one or more tumor-associated antigens, or a population of cells immunized in vitro with a tumor antigen and administered to the subject. In various embodiments, the immune checkpoint inhibitor is an antibody or antigen binding fragment thereof which binds PD-1, PD-L1, CTLA-4, LAG3, or TIM3. In various embodiments, the immune effector cell comprises a chimeric antigen receptor (CAR) molecule which binds to a tumor antigen.

In certain aspects, described herein is an immunomodulatory fusion protein comprising: an IL-2; an IL-12; a LAIR2 collagen-binding domain, wherein LAIR2 comprises at least 80% identity to the amino acid sequence as set forth in SEQ ID NO: 15; and an albumin; wherein the albumin comprises at least about 80% sequence identity to the amino acid sequence as set forth in SEQ ID NO: 16-18.

### BRIEF DESCRIPTION OF THE DRAWINGS

FIG. 1 illustrates exemplary bi-functional linear fusion collagen-localized immunomodulatory constructs comprising an IL-12, a collagen-binding domain, an albumin, and an IL-2.

FIGS. 2A-2F are graphs showing recombinant proteins purified with NiNTA resin and evaluated for product quality using analytical size exclusion chromatography (SEC). FIG. 2A shows the SEC profile of the 12-MSA-Lum-MSA-2 construct (HMW=19%; Main=79%; LMW=2%). FIG. 2B shows the SEC profile of the 12-Lum-MSA-2 construct (HMW=9%; Main=90%; LMW=1%). FIG. 2C shows the SEC profile of the 12-MSA-Lum-2 construct (HMW=34%; Main=64%; LMW=2%). FIG. 2D shows the SEC profile of the 12-MSA-LAIR-MSA-2 construct (HMW=11%; Main=89%; LMW=0%). FIG. 2E shows the SEC profile of the 12-LAIR-MSA-2 construct (HMW=11%; Main=89%; LMW=1%). FIG. 2F shows the SEC profile of the 12-MSA-LAIR-2 construct (HMW=16%; Main=84%; LMW=0%).

FIG. 3 is a bar graph showing the production yield and product quality of various constructs. The production yield and product quality (percentage of the Main peak) are highest when only a single MSA is present in the construct and such MSA was placed between the collagen binding domain (Lumican or LAIR) and IL-2

FIGS. 4A-4B are graphs showing binding of bi-functional linear fusion immunomodulatory constructs comprising a collagen-binding domain to collagen as a function of concentration. Binding was determined by ELISA. FIG. 4A shows a construct comprising LAIR (e.g., 12-MSA-LAIR-MSA-2 construct) effected higher affinity binding to collagen compared to a construct comprising Lumican (e.g., 12-MSA-Lum-MSA-2 construct). Furthermore, placing Lumican between MSA and IL-2 (e.g., 12-MSA-Lum-2 construct) enabled higher affinity binding to collagen than placing Lumican between MSA and IL-2. FIG. 4B shows three constructs comprising LAIR, each comprising a different spacer between LAIR and IL-2, MSA, ABD, and MSA_Mut1-2, effected comparable level of collagen binding. The MSA_Mut1-2 comprises an H464Q mutation which abrogates FcRn binding.

FIGS. 5A-5B are graphs showing IL-2 cytokine activity of various constructs is maintained in the presence of collagen. IL-2 bioactivity was measured for: (1) IL-2 alone, (2) IL-12 alone, (3) a combination of an IL-2 mono-functional linear construct comprising a collagen-binding domain and an IL-12 mono-functional linear construct comprising a collagen-binding domain, and (4) two bi-functional linear constructs each comprising a collagen-binding domain: 12-Lum-MSA-2 and 12-LAIR-MSA-2. FIG. 5A shows absorbance readings of the constructs on normal tissue-culture plates. FIG. 5B shows absorbance readings of the constructs on collagen I (Corning) coated plates.

FIGS. 5C-5D are graphs showing IL-2 cytokine activity of various constructs is maintained in the presence of collagen. IL-2 bioactivity was measured for three bi-functional linear constructs comprising a collagen-binding domain: (1) 12-LAIR-MSA-2, (2) 12-LAIR-MSA-2, and (3) 12-LAIR-MSA H464Q-2 which comprises an H464Q mutation which abrogates FcRn binding. FIG. 5C shows absorbance readings of the constructs on normal tissue-culture plates. FIG. 5D shows absorbance readings of the constructs on collagen I (Corning) coated plates.

FIGS. 6A-6B are graphs showing IL-12 activity of various constructs is maintained in the presence of collagen. IL-12 bioactivity was measured for: (1) IL-2 alone, (2) IL-12 alone, (3) a combination of an IL-2 mono-functional linear construct comprising a collagen-binding domain and an IL-12 mono-functional linear construct comprising a collagen-binding domain, and (4) two bi-functional linear constructs each comprising a collagen-binding domain: 12-Lum-MSA-2 and 12-LAIR-MSA-2. FIG. 6A shows absorbance readings of the constructs on normal tissue-culture plates. FIG. 6B shows absorbance readings of the constructs on collagen I (Corning) coated plates.

FIG. 7A is a graph of a tumor growth curve (mean tumor volume over time) showing the tumor volume growth of C57BL/6 mice inoculated with B16F10 cells and subsequently treated with intratumoral injections on days 0 and 6 with 100 pmol of: (1) PBS, (2) a combination of an IL-2 mono-functional linear construct comprising an MSA (MSA-2) and an IL-12 mono-functional linear construct comprising an MSA (12-MSA), (3) a combination of an IL-2 mono-functional linear construct comprising an MSA and a collagen-binding domain (LAIR-MSA-2) and an IL-12 mono-functional linear construct comprising an MSA and collagen-binding domain (12-MSA-LAIR), (4) a bi-functional linear constructs comprising MSA and a collagen-binding domain 12-Lum-MSA-2, and (5) a bi-functional linear constructs comprising MSA and a collagen-binding domain 12-LAIR-MSA-2.

FIG. 7B is a graph showing the percent change in body weight of C57BL/6 mice inoculated with B16F10 cells and subsequently treated with intratumoral injections on days 0 and 6 with 100 pmol of: (1) PBS, (2) a combination of an IL-2 mono-functional linear construct comprising an MSA (MSA-2) and an IL-12 mono-functional linear construct comprising an MSA (12-MSA), (3) a combination of an IL-2 mono-functional linear construct comprising an MSA and a collagen-binding domain (LAIR-MSA-2) and an IL-12 mono-functional linear construct comprising an MSA and collagen-binding domain (12-MSA-LAIR), (4) a bi-functional linear constructs comprising MSA and a collagen-binding domain 12-Lum-MSA-2, and (5) a bi-functional linear constructs comprising MSA and a collagen-binding domain 12-LAIR-MSA-2.

FIGS. 8A-8B is a graph of a tumor growth curve (mean tumor volume over time) showing the dose-response therapeutic efficacy of a bi-functional linear constructs comprising MSA and a collagen-binding domain, 12-LAIR-MSA-2, in a dual-flank inoculated subcutaneous Bl6F10 melanoma syngeneic model in C57BL/6 mice. The bi-functional linear constructs 12-LAIR-MSA-2, at all dose levels tested, effected significantly tumor growth inhibition, both in the treated tumor (FIG. 8A) and the untreated tumor (FIG. 8B), demonstrating abscopal effect.

FIGS. 9A-9C show the efficacy and toxicity various bi-functional constructs in the Bl6F10 mouse model. C57BL/6 mice were inoculated with Bl6F10 cells and treated with intratumoral injections of 400 pmol of (1) PBS control, (2) 12-LAIR-MSA-2, (3) 12-LAIR-MSA H464Q-2, (4) 12-LAIR-ABD-2, and (5) 12-Lum-MSA-2. FIG. 9A is a graph of a tumor growth curve (mean tumor volume over time) showing all bi-functional constructs tested effected significant tumor growth inhibition compared to the PBS control group. FIG. 9B is a graph of survival showing extended survival of the animals treated with intratumoral injections of bi-functional constructs compared to PBS control group. FIG. 9C is a graph of percent change in body weight showing all bi-functional constructs tested demonstrated good safety profile reflected by the lack of body weight loss.

FIGS. 10A-10C show the efficacy and toxicity of 12-LAIR-MSA-2 in combination with checkpoint inhibitors anti-PD1 or anti-CTLA. C57BL/6 mice were inoculated with Bl6F10 cells and treated with intratumoral (IT) injections of PBS or 400 pmol of 12-LAIR-MSA-2 and intraperitoneal (IP) injections of isotype control (Rat IgG2a), anti-PD1 (clone RMP1-14), or anti-CTLA4 (9D9) as indicated. FIGS. 10A-10B show treatment with either anti-PD1 or anti-CTLA4 alone did not affect tumor growth inhibition, treatment with bi-functional construct 12-LAIR-MSA-2 alone resulted in significant tumor growth inhibition, and the anti-tumor activity of 12-LAIR-MSA-2 was further enhanced by the combination with either anti-PD1 or anti-CTLA4. FIG. 10C shows the addition of either anti-PD1 or anti-CTLA4 to bi-functional construct 12-LAIR-MSA-2 did not result in additional weight loss compared to treatment with 12-LAIR-MSA-2 alone.

FIG. 11A is a graph of a tumor growth curve (mean tumor volume over time) showing the tumor volume growth of C57BL/6 mice inoculated with MC38 cells and subsequently treated with intratumoral injections on days 0 and 6 with PBS or 12-LAIR-MSA-2.

FIG. 11B is a graph showing the percent change in body weight of C57BL/6 mice inoculated with MC38 cells and subsequently treated with intratumoral injections on days 0 and 6 with PBS or 12-LAIR-MSA-2.

FIG. 12A is a graph of a tumor growth curve (mean tumor volume over time) showing the tumor volume growth of C57BL/6 mice inoculated with MC38 cells and subsequently treated with intratumoral injections on days 0 and 6 with intratumoral injections of indicated doses of test articles (PBS, 12-LAIR-MSA-2, 12-LAIR-ABD-2, and 12-Lum-MSA-2) on days 0 and 6. Mice were treated with intraperitoneal injections of isotype control (Rat IgG2a) or anti-PD1 (clone RMP1-14) BIW for three weeks if indicated.

FIG. 12B is a graph showing the percent change in body weight of C57BL/6 mice inoculated with MC38 cells and subsequently treated with intratumoral injections on days 0 and 6 intratumoral injections of indicated doses of test articles (PBS, 12-LAIR-MSA-2, 12-LAIR-ABD-2, and 12-Lum-MSA-2) on days 0 and 6. Mice were treated with intraperitoneal injections of isotype control (Rat IgG2a) or anti-PD1 (clone RMP1-14) BIW for three weeks if indicated.

FIG. 13A is a graph of a tumor growth curve (mean tumor volume over time) showing the tumor volume growth of BALB/c mice inoculated with CT6 cells and subsequently treated with intratumoral injections on days 0 and 6 with PBS or 12-LAIR-MSA-2.

FIG. 13B is a graph showing the percent change in body weight of BALB/c mice inoculated with CT26 cells and subsequently treated with intratumoral injections on days 0 and 6 with PBS or 12-LAIR-MSA-2.

FIG. 14A is a graph of a tumor growth curve (mean tumor volume over time) showing the tumor volume growth of BALB/c mice inoculated with CT26 cells and subsequently treated with intratumoral injections on days 0 and 6 with intratumoral injections of indicated doses of test articles (PBS, 12-LAIR-MSA-2, 12-LAIR-ABD-2, and 12-Lum-MSA-2) on days 0 and 6. Mice were treated with intraperitoneal injections of isotype control (Rat IgG2a) or anti-PD1 (clone RMP1-14) BIW for three weeks if indicated.

FIG. 14B is a graph showing the percent change in body weight of BALB/c mice inoculated with CT26 cells and subsequently treated with intratumoral injections on days 0 and 6 intratumoral injections of indicated doses of test articles (PBS, 12-LAIR-MSA-2, 12-LAIR-ABD-2, and 12-Lum-MSA-2) on days 0 and 6. Mice were treated with intraperitoneal injections of isotype control (Rat IgG2a) or anti-PD1 (clone RMP1-14) BIW for three weeks if indicated.

FIG. 15A is a graph showing levels of 12-LAIR-MSA-2 in the serum of C57BL/6 mice that were inoculated on the right rear flank with Bl6F10 cells. 7 days post-inoculation (day 0), mice were randomized into treatment groups (n=10), and the mice were treated with either intravenous or intratumoral injections of 400 pmol of PBS control or 12-LAIR-MSA-2. Two hours or 24 hours after administration, the amount of 12-LAIR-MSA-2 in the serum was measured.

FIG. 15B is a graph showing levels of interferon gamma (INF-γ) either 2 h or 24 h after administration of the 12-LAIR-MSA-2 fusion protein by IT or IV administration.

FIG. 15C is a graph showing levels of IP-10 either 2 h or 24 h after administration of the 12-LAIR-MSA-2 fusion protein by IT or IV administration.

FIG. 15D is a graph showing levels of MCP-1 either 2 h or 24 h after administration of the 12-LAIR-MSA-2 fusion protein by IT or IV administration.

FIG. 15E Is a graph showing the efficacy of treatment, as measured by survival, in mice that were administered the 12-LAIR-MSA-2 fusion protein by IT administration as compared to IV administration.

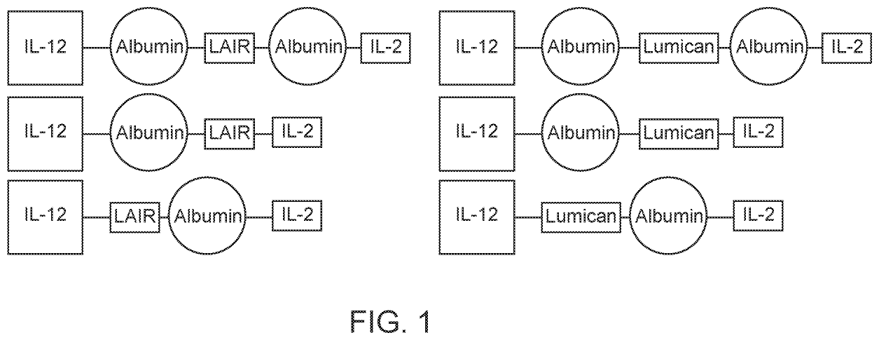

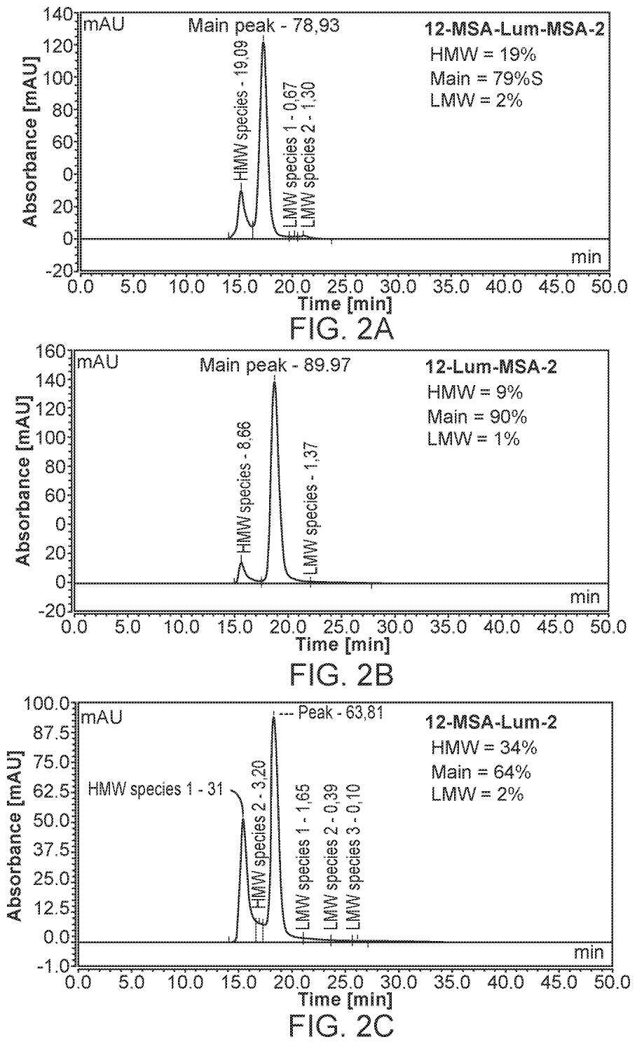

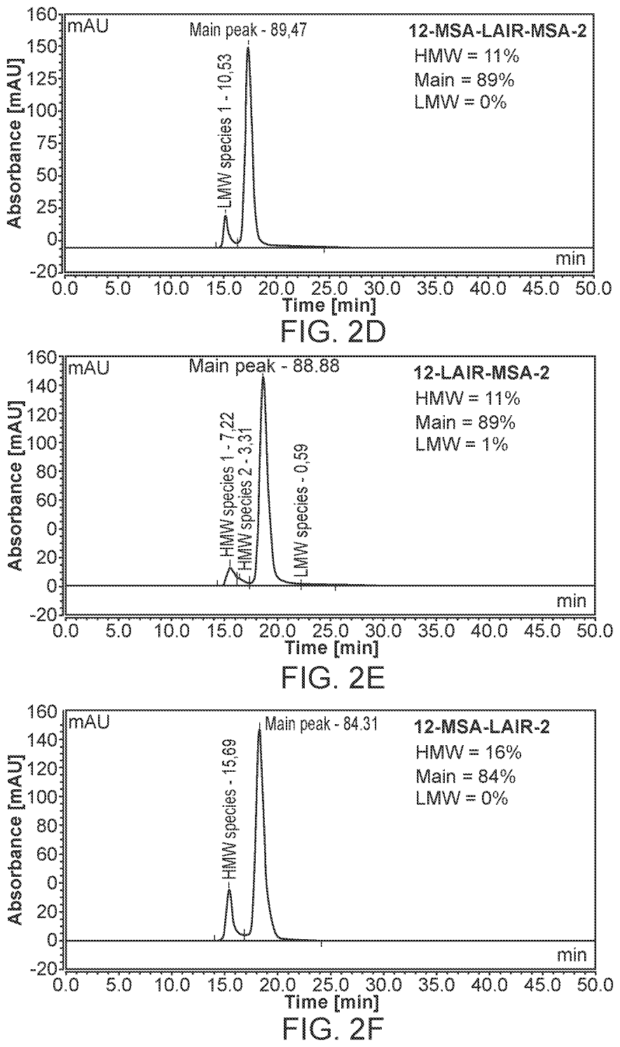

### DETAILED DESCRIPTION

Cytokines that amplify and coordinate immune cell responses for tumor control can robustly synergize with other immunotherapies. Two such cytokines are interleukin-2 (IL-2) and IL-12, which expand and stimulate T cells and natural killer (NK) cells to mediate antitumor immunity. Despite their promising therapeutic effects, in some embodiments, dose-limiting toxicity curbs the efficacy and the clinical translation of these cytokine therapies.

Ultimately, a cytokine's therapeutic index could be improved by localizing its effects to the tumor and away from healthy tissue. However, even when administered directly into a tumor, cytokines rapidly escape and enter systemic circulations, thus failing to fully address issues of toxicity and limited efficacy. The compounds described herein, in some embodiments, when injected into the tumor imiting systemic dissemination while prolonging and localizing their therapeutic antitumor activity, thereby improving efficacy while improving safety profile. In some embodiments, the compounds bind to the collagen, which is abundantly expressed and present in many tumor types.

Bi-Functional Linear Fusion Construct

To devise collagen-binding cytokines, IL-2 and IL-12 were combined in a single fusion protein with a collagen-binding protein.

When administered intratumorally, bi-functional linear immodulatory fusion proteins with a collagen-binding domain, an IL-2, and an IL-12 demonstrated reduced systemic exposure and improved therapeutic index compared to the administration of either a linear immodulatory fusion proteins with a collagen-binding domain and an IL-2, or a linear immodulatory fusion proteins with a collagen-binding domain and an IL-12. In some embodiments, the reduced systemic exposure results in a reduced toxicity or an improved therapeutic index. When administered intratumorally, bi-functional linear immodulatory fusion proteins with a collagen-binding domain, an IL-2, and an IL-12 demonstrated reduced systemic exposure compared to the combined administration of either immodulatory fusion proteins with a collagen-binding domain and a IL-2, and immodulatory fusion proteins with a collagen-binding domain and an IL-12. In some embodiments, the reduced systemic exposure results in a reduced toxicity or an improved therapeutic index.

Intratumoral Retention

Several factors dictate the intratumoral retention of a cytokine fusion protein: collagen-binding affinity, collagen concentration, size-dependent escape by diffusion or convection, and cytokine receptor-mediated consumption. Affinity to collagen and increased molecular weight contribute the intratumoral retention and systemic distribution of collagen binding fusion proteins. In some embodiments, increasing the affinity to collagen or increasing the molecular weight of a collagen-binding immunomodulatory molecule will increase intratumoral retention and decrease systemic distribution, thereby providing a therapeutic effect of a composition comprising the immunomodulatory fusion protein administered to a subject.

Accordingly, provided herein are immunomodulatory fusions to domains with specific affinity for collagen, in some embodiments, leading to greater retention within the particular collagen-rich tumors. In some aspects described herein, the immunomodulatory fusion proteins comprise an IL-2, an IL-12, and a collagen-binding domain, wherein the collagen-binding domain increases tumor retention and reduces systemic exposure to IL-2 and IL-12 following intratumoral administration in a subject, thereby reducing treatment-related toxicity.

Unless the context indicates otherwise, it is specifically intended that the various features described herein can be used in any combination. Moreover in some embodiments, any feature or combination of features set forth herein can be excluded or omitted. To illustrate, if the specification states that a complex comprises components A, B, and C, it is specifically intended that any of A, B, or C, or a combination thereof, can be omitted and disclaimed singularly or in any combination.

### Definitions

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise.

As used herein, “and/or” reder to and encompasses any and all possible combinations of one or more of the associated listed items, as well as the lack of combinations when interpreted in the alternative (“or”). Moreover, any feature or combination of features set forth herein can be excluded or omitted.

Ther term “about” as used herein when referring to a measurable value such as an amount of a compound or agent, dose, time, temperature, and the like, is meant to encompass variations of ±10%, ±5%, ±1%, ±0.5%, or even ±0.1% of the specified amount.

The terms “polypeptide,” “protein” or “peptide” refer to any chain of amino acid residues, regardless of its length or post-translational modification (e.g., glycosylation or phosphorylation).

The term “fusion protein” as used herein, refers to a protein that is created by joining two or more elements, components, or domains and/or polypeptides to create a larger polypeptide. As used herein, the terms “linked,” “operably linked,” “fused” or “fusion”, are used interchangeably, and refers to the joining together of two or more elements, components, domains and/or polypeptides within a fusion protein that allow for at least one element, component, domain and/or polypeptide to have at least a portion of the biological function or cellular activity when expressed in the fusion protein as when expressed in its natural state and/or without the linkage. The joining together of the two more elements or components or domains can be performed by whatever means known in the art including chemical conjugation, noncovalent complex formation or recombinant means. Methods of chemical conjugation (e.g., using heterobifunctional crosslinking agents) are known in the art. Thus, the elements, components, domains and/or polypeptides can be joined by covalent bonds (e.g., peptide bonds) or non-covalent bonds. The elements, components, domains and/or polypeptides can be joined by peptide bond formation in the ribosome during translation or post-translationally.

Unless otherwise defined, all technical and scientific terms used herein have the same meaning as commonly understood by one of ordinary skill in the art. The terminology used in the description herein is for the purpose of describing particular embodiments only and is not intended to be limiting.

Immunomodulatory Fusion Protein

As used herein, the term “immunomodulatory fusion protein” refers to a polypeptide comprising a collagen-binding domain operably linked to an IL-2 and IL-12. In some embodiments, the collagen binding domain is operably linked to the IL-2 and IL-12 by a linear polypeptide spacer. In some embodiments, the collagen binding domain is operably linked to the IL-2 and IL-12 by a linear polypeptide spacer. In some embodiments, the collagen binding domain is operably linked to the IL-2 and IL-12 by a linker.

In some aspects, the disclosure provides an immunomodulatory fusion protein comprising a collagen-binding domain is operably linked to an IL-2 and IL-12. In some aspects, the disclosure provides an immunomodulatory fusion protein comprising a collagen-binding domain is operably linked to an IL-2 and IL-12 by a linear polypeptide spacer. In some embodiments, the immunomodulatory fusion protein further comprises a linker. In some embodiments, the immunomodulatory fusion protein further comprises a plurality of linkers.

I. Collagen-Binding Domains

In some embodiments, the disclosure provides immunomodulatory fusion proteins comprising a collagen-binding domain. In some embodiments, the collagen-binding domain has a MW of about 5-1,000 kD, about 5-100 kDa, about 10-80 kDa, about 20-60 kDa, about 30-50 kDa, or about 10 kDa, about 20 kDa, about 30 kDa, about 40 kDa, about 50 kDa, about 60 kDa, about 70 kDa, about 80 kDa, about 90 kDa or about 100 kDa. In some embodiments, the collagen-binding domain is about 5 kDa, about 10 kDa, about 20 kDa, about 30 kDa, about 40 kDa, about 50 kDa, about 60 kDa, about 70 kDa, about 80 kDa, about 90 kDa, about 100 kDa, about 150 kDa, about 200 kDa, about 300 kDA, about 400 kDa, about 500 kDa, about 600 kDa, about 700 kDa, about 800 kDa, about 900 kDa or about 1,000 kDa. In some embodiments, the collagen-binding domain is about 30 kDa. In some embodiments, the collagen-binding domain is about 40 kDa.

In some embodiments, the collagen-binding domain is about 10-350, about 10-300, about 10-250, about 10-200, about 10-150, about 10-100, about 10-50, or about 10-20 amino acids in length. In some embodiments, the collagen-binding domain is about 10 amino acids in length. In some embodiments, the collagen-binding domain is about 15 amino acids in length. In some embodiments, the collagen-binding domain is about 20 amino acids in length. In some embodiments, the collagen-binding domain is about 30 amino acids in length. In some embodiments, the collagen-binding domain is about 40 amino acids in length. In some embodiments, the collagen-binding domain is about 50 amino acids in length. In some embodiments, the collagen-binding domain is about 60 amino acids in length. In some embodiments, the collagen-binding domain is about 70 amino acids in length. In some embodiments, the collagen-binding domain is about 80 amino acids in length. In some embodiments, the collagen-binding domain is about 90 amino acids in length. In some embodiments, the collagen-binding domain is about 100 amino acids in length. In some embodiments, the collagen-binding domain is about 120 amino acids in length. In some embodiments, the collagen-binding domain is about 150 amino acids in length. In some embodiments, the collagen-binding domain is about 200 amino acids in length. In some embodiments, the collagen-binding domain is about 250 amino acids in length. In some embodiments, the collagen-binding domain is about 300 amino acids in length. In some embodiments, the collagen-binding domain is about 350 amino acids in length.

In some embodiments, the collagen-binding domain comprises one or more (e.g., two, three, four, five, six, seven, eight, nine, ten or more) leucine-rich repeats which bind collagen. In some embodiments, the collagen-binding domain comprises a proteoglycan. In some embodiments, the collagen-binding domain comprises a proteoglycan, wherein the proteoglycan is selected from the group consisting of: decorin, biglycan, testican, bikunin, fibromodulin, lumican, chondroadherin, keratin, ECM2, epiphycan, asporin, PRELP, keratocan, osteoadherin, opticin, osteoglycan, nyctalopin, Tsukushi, podocan, podocan-like protein 1 versican, perlecan, nidogen, neurocan, aggrecan, and brevican.

In some embodiments, the collagen-binding domain comprises a class I small leucine-rich proteoglycan (SLRP). In some embodiments, the collagen-binding domain comprises a class II SLRP. In some embodiments, the collagen-binding domain comprises a class III SLRP. In some embodiments, the collagen-binding domain comprises a class IV SLRP. In some embodiments, the collagen-binding domain comprises a class V SLRP. Further description of SLRP classes is disclosed in Schaefer & Iozzo (2008) J Biol Chem 283(31):21305-21309, which is incorporated herein by reference it its entirety.

In some embodiments, the collagen-binding domain comprises one or more leucine-rich repeats from a human proteoglycan Class II member of the small leucinerich proteoglycan (SLRP) family. In some embodiments, the SLRP is selected from lumican, decorin, biglycan, fibromodulin, keratin, epiphycan, asporin and osteoglycin.

The term “k_{d}” (sec⁻¹), as used herein, refers to the dissociation rate constant of a particular protein-protein interaction. This value is also referred to as the k_{off} value.

The term “kₐ” (M⁻¹×sec⁻¹), as used herein, refers to the association rate constant of a particular protein-protein interaction. This value is also referred to as the kₒₙ value.

The term “K_{D}” (M), as used herein, refers to the dissociation equilibrium constant of a particular protein-protein interaction. K_{D}=k_{d}/kₐ. In some embodiments, the affinity of a protein (e.g., binding domain) is described in terms of the K_{D} for an interaction between two proteins. For clarity, as known in the art, a smaller K_{D} value indicates a higher affinity interaction, while a larger K_{D} value indicates a lower affinity interaction.

In some embodiments, the collagen-binding domain binds collagen (e.g., collagen type 1 or type 3) with a binding affinity K_{D} value of 0.1-1,000 nM as measured by a suitable method known in the art for determining protein binding affinity, e.g., by ELISA, surface plasmon resonance (BIAcore), FACS analysis, etc. In some embodiments, the collagen-binding domain binds collagen with a binding affinity K_{D} value of 0.1-1.0 nM, 1.0-10 nM, 10-20 nM, 20-30 nM, 30-40 mM, 40-50 nM, 50-60 nM, 70-80 nM, 90-100 nM, 10-50 nM, 50-100 nM, 100-1,000, or 1,000-10,000 nM as determined by a suitable method known in the art. In some embodiments, the immunomodulatory fusion protein binds collagen with a binding affinity K_{D} value of 0.1-1.0 nM, 1.0-10 nM, 10-20 nM, 20-30 nM, 30-40 mM, 40-50 nM, 50-60 nM, 70-80 nM, 90-100 nM, 10-50 nM, 50-100 nM, 100-1,000, or 1,000-10,000 nM as determined by a suitable method known in the art. In some embodiments, the collagen-binding domain binds trimeric peptides containing repeated GPO triplets. In some embodiments, the collagen-binding domain binds common collagen motifs in a hydroxyproline-dependent manner.

### A. Lumican

Lumican, also known as LUM, is an extracellular matrix protein that, in humans, is encoded by the LUM gene on chromosome 12 (Chakravarti et al., (1995) Genomics 27(3):481-488). Lumican is a proteoglycan Class II member of the small leucine-rich proteoglycan (SLRP) family that includes decorin, biglycan, fibromodulin, keratocan, epiphycan, and osteoglycin (Iozzo & Schaefer (2015) Matrix Biology 42: 11-55). Lumican is a stable protein that binds specifically to collagen types I and IV.

Lumican has a molecular weight of about 40 kDa and has four major intramolecular domains: 1) a signal peptide of 16 amino acid residues, 2) a negatively-charged N-terminal domain containing sulfated tyrosine and disulfide bond(s), 3) ten tandem leucine-rich repeats allowing lumican to bind to collagen, and 4) a carboxyl terminal domain of 50 amino acid residues containing two conserved cysteines 32 residues apart. Kao et al., (2006) Experimental Eye Research 82(1):3-4). There are four N-linked sites within the leucine-rich repeat domain of the protein core that can be substituted with keratan sulfate. The core protein of lumican (like decorin and fibromodulin) is horseshoe shaped. This enables it bind to collagen molecules within a collagen fibril, thus helping keep adjacent fibrils apart Scott (1996) Biochemistry 35(27): 8795-8799.

In some embodiments, the collagen-binding domain comprises a class II small leucine-rich proteoglycan (SLRP). Further description of SLRP classes is disclosed in Schaefer & Iozzo (2008) J Biol Chem 283(31):21305-21309, which is incorporated herein by reference it its entirety. In some embodiments, the collagen-binding domain comprises one or more leucine-rich repeats from a human proteoglycan Class II member of the small leucinerich proteoglycan (SLRP) family. In some embodiments, the SLRP is lumican. In some embodiments, the lumican is human lumican. In some embodiments, lumican comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 11, or a portion thereof.

In some embodiments, the lumican is a variant comprising one or more amino acid substitutions, additions or deletions, optionally two, three, four, five, six, seven, eight, nine, ten or more amino acid substitutions, additions or deletions relative to a lumican protein comprising the amino acid sequence of SEQ ID NO: 11. In some embodiments, the lumican variant has increased binding affinity to collagen relative to a collagen binding affinity of a lumican protein comprising the amino acid sequence of SEQ ID NO: 11. In some embodiments, the lumican variant has decreased binding affinity to collagen relative to a collagen binding affinity of a lumican protein comprising the amino acid sequence of SEQ ID NO: 11.

### B. LAIR1 and LAIR2

Leukocyte-Associated Immunoglobulin-Like Receptors (LAIR- and LAIR-2) Leukocyte-associated lg-like receptor (LAIR)-1 is a collagen-receptor that inhibits immune cell function upon collagen binding. Next to LAIR-I, the human genome encodes LAIR-2, a soluble homolog. Human (h) LAIR-I is expressed on the majority of PBMC and thymocytes (Maasho et al., (2005) Mal Immunol 42: 1521-1530). Cross-linking of LAIR-1 by mAbs in vitro delivers a potent inhibitory signal that is capable of inhibiting immune cell function. Collagens are known to be natural, high-affinity ligands for the LAIR molecules. Interaction of hLAIR-1 with collagens directly inhibits immune cell activation in vitro (Meyaard et al., (1997) Immunity 7:283-290; Poggi (1998) Eur J Immunol 28:2086-2091; Van der Vuurst de Vries et al., (1999) Eur J Immunol 29:3160-3167; Lebbink et al., (2006) J Exp Med 203:1419-1425).

In some embodiments, the collagen-binding domain comprises a human type I glycoprotein having an Ig-like domain, or an extracellular portion thereof which binds collagen. In some embodiments, the type I glycoprotein competes with lumican for binding for binding to collagen type I. In some embodiments, the human type I glycoprotein is selected from LAIR, LAIR1, and LAIR2.

In some embodiments, the human type I glycoprotein is LAIR1. In some embodiments, the LAIR1 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 13, or a portion thereof. In some embodiments, the human type I glycoprotein is LAIR1 and the collagen-binding domain comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to amino acid residues 22-122 of the amino acid sequence as set forth in SEQ ID NO: 13, or a portion thereof.

In some embodiments, the human type I glycoprotein is LAIR1. In some embodiments, the LAIR1 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 14, or a portion thereof.

In some embodiments, the LAIR comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 12, or a portion thereof.

In some embodiments, the LAIR1 is a variant comprising one or more amino acid substitutions, additions or deletions, optionally two, three, four, five, six, seven, eight, nine, ten or more amino acid substitutions, additions or deletions relative to a LAIR1 protein comprising the amino acid sequence of SEQ ID NO: 13. In some embodiments, the LAIR1 variant has increased binding affinity to collagen relative to a collagen binding affinity of a LAIR1 protein comprising the amino acid sequence of SEQ ID NO: 13. In some embodiments, the LAIR1 variant has decreased binding affinity to collagen relative to a collagen binding affinity of a LAIR1 protein comprising the amino acid sequence of SEQ ID NO: 13.

In some embodiments, the LAIR1 is a variant comprising one or more amino acid substitutions, additions or deletions, optionally two, three, four, five, six, seven, eight, nine, ten or more amino acid substitutions, additions or deletions relative to a LAIR1 protein comprising the amino acid sequence of SEQ ID NO: 14. In some embodiments, the LAIR1 variant has increased binding affinity to collagen relative to a collagen binding affinity of a LAIR1 protein comprising the amino acid sequence of SEQ ID NO: 14. In some embodiments, the LAIR1 variant has decreased binding affinity to collagen relative to a collagen binding affinity of a LAIR1 protein comprising the amino acid sequence of SEQ ID NO: 14.

In some embodiments, the LAIR is a variant comprising one or more amino acid substitutions, additions or deletions, optionally two, three, four, five, six, seven, eight, nine, ten or more amino acid substitutions, additions or deletions relative to a LAIR protein comprising the amino acid sequence of SEQ ID NO: 12. In some embodiments, the LAIR1 variant has increased binding affinity to collagen relative to a collagen binding affinity of a LAIR protein comprising the amino acid sequence of SEQ ID NO: 12. In some embodiments, the LAIR variant has decreased binding affinity to collagen relative to a collagen binding affinity of a LAIR protein comprising the amino acid sequence of SEQ ID NO: 12.

In some embodiments, the human type I glycoprotein is LAIR2. In some embodiments, the LAIR2 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NO: 15, or a portion thereof.

In some embodiments, the LAIR2 is a variant comprising one or more amino acid substitutions, additions or deletions, optionally two, three, four, five, six, seven, eight, nine, ten or more amino acid substitutions, additions or deletions relative to a LAIR2 protein comprising the amino acid sequence of SEQ ID NO: 15. In some embodiments, the LAIR2 variant has increased binding affinity to collagen relative to a collagen binding affinity of a LAIR2 protein comprising the amino acid sequence of SEQ ID NO: 15. In some embodiments, the LAIR2 variant has decreased binding affinity to collagen relative to a collagen binding affinity of a LAIR2 protein comprising the amino acid sequence of SEQ ID NO: 15.

TABLE 1
Exemplary sequences for collagen-binding domains

| SEQ ID | | |
|---|---|---|
| NO: | Description | Sequence |
| 11 | Lumican | MSLSAFTLFLALIGGTSGQYYDYDFPLSIYGQSSPNCAPECNCP |
| | | ESYPSAMYCDELKLKSVPMVPPGIKYLYLRNNQIDHIDEKAFE |
| | | NVTDLQWLILDHNLLENSKIKGRVFSKLKQLKKLHINHNNLT |
| | | ESVGPLPKSLEDLQLTHNKITKLGSFEGLVNLTFIHLQHNRLKE |
| | | DAVSAAFKGLKSLEYLDLSFNQIARLPSGLPVSLLTLYLDNNK |
| | | ISNIPDEYFKRFNALQYLRLSHNELADSGIPGNSFNVSSLVELD |
| | | LSYNKLKNIPTVNENLENYYLEVNQLEKFDIKSFCKILGPLSYS |
| | | KIKHLRLDGNRISETSLPPDMYECLRVANEVTLN |
| 12 | LAIR | MSLHPVILLVLVLCLGWKINTQEGSLPDITIFPNSSLMISQGTFV |
| | (murine) | TVVCSYSDKHDLYNMVRLEKDGSTFMEKSTEPYKTEDEFEIG |
| | | PVNETITGHYSCIYSKGITWSERSKTLELKVIKENVIQTPAPGPT |
| | | SDTSWLKTYSIYIFTVVSVIFLLCLSALLFCFLRHRQKKQGLPN |
| | | NKRQQQRPEERLNLATNGLEMTPDIVADDRLPEDRWTETWTP |
| | | VAGDLQEVTYIQLDHHSLTQRAVGAVTSQSTDMAESSTYAAII |
| | | RH |
| 13 | LAIR1 | MSPHPTALLGLVLCLAQTIHTQEEDLPRPSISAEPGTVIPLGSH |
| | (human) | VTFVCRGPVGVQTFRLERESRSTYNDTEDVSQASPSESEARFRI |
| | | DSVSEGNAGPYRCIYYKPPKWSEQSDYLELLVKETSGGPDSPD |
| | | TEPGSSAGPTQRPSDNSHNEHAPASQGLKAEHLYILIGVSVVF |
| | | LFCLLLLVLFCLHRQNQIKQGPPRSKDEEQKPQQRPDLAVDVL |
| | | ERTADKATVNGLPEKDRETDTSALAAGSSQEVTYAQLDHWA |
| | | LTQRTARAVSPQSTKPMAESITYAAVARH |
| 14 | ECD of | QEEDLPRPSISAEPGTVIPLGSHVTFVCRGPVGVQTFRLERESR |
| | LAIR1 | STYNDTEDVSQASPSESEARFRIDSVSEGNAGPYRCIYYKPPK |
| | | WSEQSDYLELLVKETSGGPDSPDTEPGSSAGPTQRPSDNSHNE |
| | | HAPASQGLKAEHLY |
| 15 | LAIR2 | MSPHLTALLGLVLCLAQTIHTQEGALPRPSISAEPGTVISPGSH |
| | (human) | VTFMCRGPVGVQTFRLEREDRAKYKDSYNVFRLGPSESEARF |
| | | HIDSVSEGNAGLYRCLYYKPPGWSEHSDFLELLVKESSGGPDS |
| | | PDTEPGSSAGTVPGTEASGFDAP |

II. Immunomodulatory Domain

The immunomodulatory fusion proteins disclosed herein comprises at least one IL-2 and at least one IL-12. In certain embodiments, the immunomodulatory fusion proteins disclosed herein comprises an IL-2, an IL-12, and a collagen-binding domain. In certain embodiments, the immunomodulatory fusion proteins disclosed herein comprises an IL-2, an IL-12, a collagen-binding domain, and at least one linear polypeptide spacer. In some embodiments, the IL-2 is operably linked to a collagen-binding domain. In some embodiments, the IL-2 is operably linked to a linear polypeptide spacer. In some embodiments, the IL-12 is operably linked to a collagen-binding domain. In some embodiments, the IL-12 is operably linked to a linear polypeptide spacer.

### A. IL-2

As used herein, “interleukin (IL)-2,” (IL-2) refers to a pleiotropic cytokine that activates and induces proliferation of T cells and natural killer (NK) cells. The biological activity of IL-2 is mediated through a multi-subunit IL-2 receptor complex (IL-2R) of three polypeptide subunits that span the cell membrane: p55 (IL-2Rα, the alpha subunit, also known as CD25 in humans), p75 (IL-2Rβ, the beta subunit, also known as CD122 in humans) and p64 (IL-2Rγ, the gamma subunit, also known as CD132 in humans).

In some embodiments, the immunomodulatory fusion protein comprises an IL-2. In some embodiments, the IL-2 is operably linked to a collagen binding domain. In some embodiments, the immunomodulatory fusion protein comprises a member of the IL-2 family operably linked to a collagen binding domain.

T cell response to IL-2 depends on a variety of factors, including: (1) the concentration of IL-2; (2) the number of IL-2R molecules on the cell surface; and (3) the number of IL-2R occupied by IL-2 (i.e., the affinity of the binding interaction between IL-2 and IL-2R (Smith, “Cell Growth Signal Transduction is Quanta!” In Receptor Activation by Antigens, Cytokines, Hormones, and Growth Factors 766:263-271, 1995)).

In some embodiments, the IL-2 is wild-type IL-2 (e.g., human IL-2 in its precursor form or mature IL-2. In some embodiments, the IL-2 is human IL-2. In some embodiments, the IL-2 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NOs: 1 or 2, or a portion thereof. In some embodiments, the IL-2 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NOs: 3 or 4, or a portion thereof.

In other embodiments, the IL-2 is a mutant human IL-2. The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as an IL-2 mutant peptide sequence, an IL-2 mutant polypeptide, IL-2 mutant protein or IL-2 mutant analog.

In some embodiments, IL-2 mutants comprise an amino acid sequence that is at least 80% identical to SEQ ID NOs: 1 or 2 that bind CD25. For example, some embodiments an IL-2 mutant has at least one mutation (e.g., a deletion, addition, or substitution of 1, 2, 3, 4, 5, 6, 7, 8, 9, 10, 11, 12, 13, 14, 15, 16, 17, 18, 19, 20, or more amino acid residues) that increases the affinity for the alpha subunit of the IL-2 receptor relative to wild-type IL-2. It should be understood that mutations identified in mouse IL-2 may be made at corresponding residues in full length human IL-2 (nucleic acid sequence (accession: NM000586); amino acid sequence (accession: P60568) or human IL-2 without the signal peptide. Accordingly, in some embodiments, the IL-2 is human IL-2. In other embodiments, the IL-2 is a mutant human IL-2. The amino acid sequence of human IL-2 (SEQ ID NO:1; full length) is found in Genbank under accession locator NP_000577.2. The amino acid sequence of mature human IL-2 is depicted in SEQ ID NO:2 (human wild-type mature). The murine (Mus musculus) IL-2 amino acid sequence is found in Genbank under accession locator (SEQ ID NO:3). The amino acid sequence of mature murine IL-2 is depicted in SEQ ID NO:4.

In certain embodiments, IL-2 is mutated such that it has an altered affinity (e.g., a lower affinity) for the IL-2R alpha receptor compared with unmodified IL-2. Site-directed mutagenesis can be used to isolate IL-2 mutants that exhibit decreased affinity binding to CD25, i.e., IL-2Rα, as compared to wild-type IL-2. Increasing the affinity of IL-2 for IL-2Rα at the cell surface will increase receptor occupancy within a limited range of IL-2 concentration, as well as raise the local concentration of IL-2 at the cell surface.

In some embodiments, the amino acid substitutions increasing IL-2Rβ binding affinity include: L80F, R81D, L85V, I86V, and I92F. In some embodiments, the amino acid substitutions that increase IL-2Rβ binding affinity include: L80F, R81D, L85V, I86V, and I92F.

TABLE 2
Exemplary sequences for IL-2

| SEQ ID | | |
|---|---|---|
| NO: | Description | Sequence |
| 1 | Human IL-2 | MYRMQLLSCIALSLALVTNSAPTSSSTKKTQLQLEHLL |
| | | LDLQMILNGINNYKNPKLTRMLTFKFYMPKKATELKH |
| | | LQCLEEELKPLEEVLNLAQSKNFHLRPRDLISNINVIVL |
| | | ELKGSETTFMCEYADETATIVEFLNRWITFCQSIISTLT |
| 2 | Human wild-type | APTSSSTKKTQLQLEHLLLDLQMILNGINNYKNPKLTR |
| | mature IL-2 | MLTFKFYMPKKATELKHLQCLEEELKPLEEVLNLAQS |
| | | KNFHLRPRDLISNINVIVLELKGSETTFMCEYADETATI |
| | | VEFLNRWITFCQSIISTLT |
| 3 | Murine IL-2 | MYSMQLASCVTLTLVLLVNSAPTSSSTSSSTAEAQQQQ |
| | | QQQQQQQQHLEQLLMDLQELLSRMENYRNLKLPRML |
| | | TFKFYLPKQATELKDLQCLEDELGPLRHVLDLTQSKSF |
| | | QLEDAENFISNIRVTVVKLKGSDNTFECQ |
| | | FDDESATVVDFLRRWIAFCQSIISTSPQ |
| 4 | Murine mature | APTSSSTSSSTAEAQQQQQQQQQQQQHLEQLLMDLQE |
| | IL-2 | LLSRMENYRNLKLPRMLTFKFYLPKQATELKDLQCLE |
| | | DELGPLRHVLDLTQSKSFQLEDAENFISNIRVTVVKLK |
| | | GSDNTFECQFDDESATVVDFLRRWIAFCQSIISTSPQ |

### B. IL-12

Interleukin-12 (IL-2) plays an important role in innate and adaptive immunity. Gately, M K et al., Annu Rev Immunol. 16: 495-521 (1998). IL-12 functions primarily as a 70 kDa heterodimeric protein consisting of two disulfide-linked p35 and p40 subunits. The precursor form of the IL-12 p40 subunit (NM 002187; P29460; also referred to as IL-12B, natural killer cell stimulatory factor 2, cytotoxic lymphocyte maturation factor 2) is 328 amino acids in length, while its mature form is 306 amino acids long. The precursor form of the IL-12 p35 subunit (NM 000882; P29459; also referred to as IL-12A, natural killer cell stimulatory factor 1, cytotoxic lymphocyte maturation factor 1) is 219 amino acids in length and the mature form is 197 amino acids long.

In some embodiments, the immunomodulatory fusion protein comprises an IL-12. In some embodiments, the immunomodulatory fusion protein comprises an IL-12 operably linked to a collagen binding domain.

In some embodiments, the IL-12 comprises IL-12A (e.g., SEQ ID NO: 6). In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12A as set forth in SEQ ID NO: 6, or a portion thereof.

In some embodiments, the IL-12 comprises IL-12A (e.g., SEQ ID NO: 8). In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12A as set forth in SEQ ID NO: 8, or a portion thereof.

In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12A as set forth in SEQ ID NO: 10, or a portion thereof.

In some embodiments, the IL-12 comprises IL-12B (e.g., SEQ ID NOs: 5). In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12B as set forth in SEQ ID NO: 5, or a portion thereof.

In some embodiments, the IL-12 comprises IL-12B (e.g., SEQ ID NO: 7). In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12B as set forth in SEQ ID NOs: 7, or a portion thereof. In some embodiments, the IL-12 comprises IL-12B (e.g., SEQ ID NO: 7).

In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12B as set forth in SEQ ID NO: 9, or a portion thereof.

In some embodiments, the IL-12 comprises both IL-12A and IL-12B. In some embodiments, the IL-12 comprises both IL-12A and IL-12B and a linker. In some embodiments, the immunomodulatory fusion protein comprises an IL-12 comprising the amino acid sequences set forth in SEQ ID NOs: 5-10. In some embodiments, the IL-12 comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of IL-12A and IL-12B as set forth in SEQ ID NOs: 5-10, or a portion thereof.

The term “IL-12 mutant” or “mutant IL-12 polypeptide” as used herein is intended to encompass any mutant forms of various forms of the IL-12 molecule including full-length IL-12, truncated forms of IL-12 and forms where IL-12 is linked to another molecule such as by fusion or chemical conjugation. The various forms of IL-12 mutants are characterized in having a at least one amino acid mutation. This mutation may involve substitution, deletion, truncation or modification of the wild-type amino acid residue normally located at that position. Mutants obtained by amino acid substitution are preferred. Unless otherwise indicated, an IL-12 mutant may be referred to herein as an IL-12 mutant peptide sequence, an IL-12 mutant polypeptide, IL-12 mutant protein or IL-12 mutant analog.

TABLE 3
Exemplary sequences for IL-12

| SEQ ID | | |
|---|---|---|
| NO: | Protein | Sequence |
| 5 | Human IL-12 p40 | MHPQQLVVSWFSLVLLASPIVAIWELEKNVYVVELDW |
| | wild-type | YPDAPGETVVLTCDTPEEDGITWTSDQSSEVLGSGKTLT |
| | (IL-12 subunit beta; | IQVKEFGDAGQYTCHKGGEALSRSLLLLHKKEDGIWST |
| | IL-12B) | DILKDQKEPKAKSFLKCEAKDYSGHFTCWWLTAISTDL |
| | | KFSVKSSRGSSDPRGVTCEAASLSAEKVSVDHREYNKY |
| | | TVECQEGSTCPAAEESLLIEVVVEAVHKLKYENYTSSFF |
| | | IRDIIKPDPPKNLQLKPLKNSRQVEVSWEYPDTWSTPHS |
| | | YFSLTFCVQVQGKNKREKKLFMDQTSAKVTCHKDANV |
| | | RVQARDRYYSSFWSEWASVSCS |
| 6 | Human IL-12 p35 | MSPLRKCLLLTSLVLLVSCSLARNLPRASPAPVTEPVQC |
| | wild-type | FNHSQTLLRAVNSELHKAIQMLAVYSCTPEEIDHEDITK |
| | (IL-12 subunit alpha; | DKTSTVKACVPLELVKNESCLASGHISFTTNGSCLASGK |
| | IL-12A) | TSFMMALCLNSIYEDLKLYQLEFKNMNAQLLMDPQRQ |
| | | IFLDQNMLSAIDELIQALNGSDVTVPQKLSLEEPDFYKIK |
| | | MKLCILLHAFRIRAVTIDRVMSYLTSS |
| 7 | Human IL-12B | IWELKKDVYVVELDWYPDAPGEMVVLTCDTPEEDGIT |
| | without signal peptide | WTLDQSSEVLGSGKTLTIQVKEFGDAGQYTCHKGGEV |
| | | LSHSLLLLHKKEDGIWSTDILKDQKEPKNKTFLRCEAK |
| | | NYSGRFTCWWLTTISTDLTFSVKSSRGSSDPQGVTCGA |
| | | ATLSAERVRGDNKEYEYSVECQEDSACPAAEESLPIEV |
| | | MVDAVHKLKYENYTSSFFIRDIIKPDPPKNLQLKPLKNS |
| | | RQVEVSWEYPDTWSTPHSYFSLTFCVQVQGKSKREKK |
| | | DRVFTDKTSATVICRKNASISVRAQDRYYSSSWSEWAS |
| | | VPCS |
| 8 | Human IL-12A | RNLPVATPDPGMFPCLHHSQNLLRAVSNMLQKARQTL |
| | without signal peptide | EFYPCTSEEIDHEDITKDKTSTVEACLPLELTKNESCLNS |
| | | RETSFITNGSCLASRKTSFMMALCLSSIYEDLKMYQVEF |
| | | KTMNAKLLMDPKRQIFLDQNMLA VIDELMQALNFNSE |
| | | TVPQKSSLEEPDFYKTKIKLCILLHAFRIRAVTIDRVMSY |
| | | LNAS |
| 9 | Murine IL-12 subunit | MWELEKDVYVVEVDWTPDAPGETVNLTCDTPEEDDIT |
| | beta | WTSDQRHGVIGSGKTLTITVKEFLDAGQYTCHKGGETL |
| | | SHSHLLLHKKENGIWSTEILKNFKNKTFLKCEAPNYSGR |
| | | FTCSWLVQRNMDLKFNIKSSSSSPDSRAVTCGMASLSA |
| | | EKVTLDQRDYEKYSVSCQEDVTCPTAEETLPIELALEAR |
| | | QQNKYENYSTSFFIRDIIKPDPPKNLQMKPLKNSQVEVS |
| | | WEYPDSWSTPHSYFSLKFFVRIQRKKEKMKETEEGCNQ |
| | | KGAFLVEKTSTEVQCKGGNVCVQAQDRYYNSSCSKW |
| | | ACVPCRVRS |
| 10 | Murine IL-12 subunit | RVIPVSGPARCLSQSRNLLKTTDDMVKTAREKLKHYSC |
| | alpha | TAEDIDHEDITRDQTSTLKTCLPLELHKNESCLATRETSS |
| | | TTRGSCLPPQKTSLMMTLCLGSIYEDLKMYQTEFQAIN |
| | | AALQNHNHQQIILDKGMLVAIDELMQSLNHNGETLRQ |
| | | KPPVGEADPYRVKMKLCILLHAFSTRVVTINRVMGYLS |
| | | SA |

III. Linear Polypeptide Spacer

In some embodiments, the linear polypeptide spacer is a polypeptide comprising “N” amino acids in length, wherein N=1-1000, 50-800, 100-600, or 200-500. In some embodiments, the linear polypeptide spacer comprises about 1 to about 100 amino acid residues. In some embodiments, the linear polypeptide space comprises more than 100 amino acid residues. In certain embodiments, the linear polypeptide spacer comprises about 1 to about 100 amino acid residues.

In some embodiments, the linear polypeptide spacer is a soluble polypeptide. In some embodiments, the linear polypeptide spacer has a molecular weight between 1 and 200 kDa. In some embodiments, the linear polypeptide spacer has a molecular weight 1-10 kDa, 10-20 kDa, 20-30 kDa, 30-40 kDa, 40-50 kDa, 50-60 kDa, 60-70 kDa, 70-80 kDa, 80-90 kDa, 90-100 kDa, 100-110 kDa, 110-120 kDa, 120-130 kDa, 130-140 kDa, 140-150 kDa, 150-160 kDa, 160-170 kDa, 170-180 kDa, 180-190 kDa, 190-200 kDa, 10-100, 100-200 kDa, 200-300 kDa, 300-400 kDa, 400-500 kDA, 500-1,000 kDa, or 100-1,000 kDa.

In certain embodiments, the linear polypeptide spacer provides a steric separation between one element of the fusion protein to another. In certain embodiments, the linear polypeptide spacer provides a steric separation between one domain of the fusion protein to another. In some embodiments, the linear polypeptide spacer between the IL-2 and the collagen-binding protein provides a steric separation such that the IL-2 retains its activity (e.g., promote receptor/ligand engagement). In some embodiments, the linear polypeptide spacer between the IL-12 and the collagen-binding protein provides a steric separation such that the IL-12 retains its activity (e.g., promote receptor/ligand engagement). In certain embodiments, the linear polypeptide spacer between the IL-2 and the collagen-binding protein and/or the IL-12 and the collagen-binding protein provides a steric separation such that the IL-2 and/or the IL-12 binds to to receptors on the same cell. In certain embodiments, the linear polypeptide spacer between the IL-2 and the collagen-binding protein and/or the IL-12 and the collagen-binding protein provides a steric separation such that the IL-2 and/or the IL-12 binds to receptors on different cells.

In some embodiments, the linear polypeptide spacer between IL-2 and the collagen-binding protein is of sufficient length or mass to reduce adsorption of the immunomodulatory domain onto collagen fibrils. In some embodiments, the linear polypeptide spacer between IL-12 and the collagen-binding protein is of sufficient length or mass to reduce adsorption of the immunomodulatory domain onto collagen fibrils. Methods for measuring adsorption are known to those of skill in the art. For example, adsorption can be measured by ellipsometry (ELM), surface plasmon resonance (SPR), optical waveguide lightmode spectroscopy (OWLS), attenuated total internal reflectance-infrared spectroscopy (ATR-IR), circular dichroism spectroscopy (CD), total internal reflectance infrared spectroscopy (TIRF), and other high resolution microscopy techniques. In some embodiments, these methods show the spatial arrangement between the domains of the immunomodulatory fusion protein.

In certain embodiments, the linear polypeptide spacer provides one of several functional benefits, including but not limited to: i) separation of IL2 and IL12 to allow both cytokines to access their receptors either on the same cell or separate cells; ii) separation of collagen from IL2 to improve the geometries of their interactions in vivo; iii) increased hydrodynamic radius of the fusion construct, thereby utilizing size exclusion to slow down the rate of burst release upon administration; and/or iv) stabilization and/or improved solubilization of domains that are relatively insoluble. In certain embodiments, the linear polypeptide spacer improves retention of the fusion product at the target tissue when administered to a subject.

In some embodiments, the linear polypeptide spacer between IL-2 and the collagen-binding protein provides sufficient molecular weight to slow or reduce diffusion from the tissue. In some embodiments, the linear polypeptide spacer between IL-12 and the collagen-binding protein provides sufficient molecular weight to slow or reduce diffusion from the tissue. Methods for measuring diffusion from the tissue are known to those of skill in the art. For example, diffusion can be measured by in vivo imagining, or via microscopy of tissue sections over time. Exemplary methods are described in at least Schmidt & Wittrup, Mol. Canc. Ther. 2009′ and Wittrup et al., Methods in Enzymol 2012, each of which is herein incorporated by reference in their entirety.

### Albumin

The term “albumin” refers to a protein having the same, or very similar three dimensional structure as human albumin (SEQ ID NO: 16) and having a long serum half-life. Exemplary albumin proteins include human serum albumin (HSA; SEQ ID NOs: 17 and 18), primate serum albumin (such as chimpanzee serum albumin), gorilla serum albumin or macaque serum albumin, rodent serum albumin (such as hamster serum albumin), guinea pig serum albumin, mouse serum albumin and rat serum albumin, bovine serum albumin (such as cow serum albumin), equine serum albumin (such as horse serum albumin or donkey serum albumin), rabbit serum albumin, goat serum albumin, sheep serum albumin, dog serum albumin, chicken serum albumin and pig serum albumin.

In some embodiments, the linear polypeptide spacer is an albumin, an albumin binder, an albumin binding domain, or an albumin mutation. In some embodiments, the linear polypeptide spacer comprises albumin, or fragments thereof. In some embodiments, the linear polypeptide spacer is human albumin. In some embodiments, the albumin is a serum albumin, for example, a human serum albumin (SEQ ID NO: 17). In some embodiments, the linear polypeptide spacer is an albumin binding domain.

In some embodiments, the linear polypeptide spacer comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of human albumin as set forth in SEQ ID NO: 16, or a portion thereof.

In some embodiments, the linear polypeptide spacer comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of human serum albumin as set forth in SEQ ID NO: 17, or a portion thereof.

In some embodiments, the linear polypeptide spacer comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of human serum albumin as set forth in SEQ ID NO: 18, or a portion thereof.

In some embodiments, the albumin is a variant comprising one or more amino acid substitutions, additions or deletions, optionally two, three, four, five, six, seven, eight, nine, ten or more amino acid substitutions, additions or deletions relative to an albumin protein comprising the amino acid sequence of SEQ ID NOs: 16-18. In some embodiments, the albumin mutation comprises at least one amino acid mutation compared to wild-type albumin. This mutation may involve substitution, deletion, truncation or modification of the wild-type amino acid residue normally located at that position.

In certain embodiments, the linear polypeptide is a serum protein binding domain. In some embodiments, the linear polypeptide spacer is an albumin binding domain. In some embodiments, the linear polypeptide spacer comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of albumin binding domain as set forth in SEQ ID NO: 19, or a portion thereof. In some embodiments, the albumin binding domain non-covalently binds to serum albumin once administered to a subject. In some embodiments, the albumin binding domain demonstrates a non-covalent means of enhancing the hydrodynamic radius of the fusion construct in situ. In certain embodiments, the albumin binding domain improves retention of the fusion construct at the target tissue when administered to a subject.

TABLE 4
Exemplary Sequences for Albumin

| SEQ ID | | |
|---|---|---|
| NO: | Description | Sequence |
| 16 | Human | MKWVTFISLLFLFSSAYSRGVFRRDAHKSEVAHRFKDLGEEN |
| | albumin | FKALVLIAFAQYLQQCPFEDHVKLVNEVTEFAKTCVADESAE |
| | | NCDKSLHTLFGDKLCTVATLRETYGEMADCCAKQEPERNECF |
| | | LQHKDDNPNLPRLVRPEVDVMCTAFHDNEETFLKKYLYEIAR |
| | | RHPYFYAPELLFFAKRYKAAFTECCQAADKAACLLPKLDELR |
| | | DEGKASSAKQRLKCASLQKFGERAFKAWAVARLSQRFPKAE |
| | | FAEVSKLVTDLTKVHTECCHGDLLECADDRADLAKYICENQD |
| | | SISSKLKECCEKPLLEKSHCIAEVENDEMPADLPSLAADFVESK |
| | | DVCKNYAEAKDVFLGMFLYEYARRHPDYSVVLLLRLAKTYE |
| | | TTLEKCCAAADPHECYAKVFDEFKPLVEEPQNLIKQNCELFEQ |
| | | LGEYKFQNALLVRYTKKVPQVSTPTLVEVSRNLGKVGSKCCK |
| | | HPEAKRMPCAEDYLSVVLNQLCVLHEKTPVSDRVTKCCTESL |
| | | VNRRPCFSALEVDETYVPKEFNAETFTFHADICTLSEKERQIKK |
| | | QTALVELVKHKPKATKEQLKAVMDDFAAFVEKCCKADDKET |
| | | CFAEEGKKLVAASQAALGL |
| 17 | Human serum | MDMRVPAQLLGLLLLWLPGARCADAHKSEVAHRFKDLGEEN |
| | albumin | FKALVLIAFAQYLQQCPFEDHVKLVNEVGEFAKTCVADESAE |
| | | NCDKSLHTLFGDKLCTVATLRETYGEMADCCAKQEPERNECF |
| | | LQHKDDNPNLPRLVRPEVDVMCTAFHDNEETFLKKYLYEIAR |
| | | HPYFYAPELLFFAKRYKAAFTECCQAADKAACLLPKLDELRD |
| | | EGKASSAKQRLKCASLQKFGERAFKAWAVARLSQRFPKAEF |
| | | AEVSKLVTDLTKVHTECCHGDLLECADDRADLAKYICENQDS |
| | | ISSKLKECCEKPLLEKSHCIAEVENDEMPADLPSLAADFVESK |
| | | DVCKNYAEAKDVFLGMFLYEYARRHPDYSWLLLRLAKTYET |
| | | TLEKCCAAADPHECYAKVFDEFKPLVEEPQNLIKQNCELFEQL |
| | | GEYKFQNALLVRYTKKVPQVSTPTLVEVSRNLGKVGSKCCK |
| | | HPEAKRMPCAEDYLSWLNQLCVLHEKTPVSDRVTKCCTESLV |
| | | NRRPCFSALEVDEGYVPKEFNAETFTFHADICTLSEKERQIKK |
| | | QTALVELVKHKPKATKEQLKAVMDDFAAFVEKCCKADDKET |
| | | CFAEEGKKLVAASQAALGLGGGSAPTSSSTKKTQLQLEHLLL |
| | | DLQMILNGINNYKNPKLTRMLTFKFYMPKKATELKHLQCLEE |
| | | ELKPLEEVLNLAQSKNFHLRPRDLISNINVIVLELKGSETTFMC |
| | | EYADETATIVEFLNRWITFCQSIISTLGGGGS |
| 18 | Mature HSA | DAHKSEVAHRFKDLGEENFKALVLIAFAQYLQQCPFEDHVKL |
| | | VNEVTEFAKTCVADESAENCDKSLHTLFGDKLCTVATLRETY |
| | | GEMADCCAKQEPERNECFLQHKDDNPNLPRLVRPEVDVMCT |
| | | AFHDNEETFLKKYLYEIARRHPYFYAPELLFFAKRYKAAFTEC |
| | | CQAADKAACLLPKLDELRDEGKASSAKQRLKCASLQKFGER |
| | | AFKAWAVARLSQRFPKAEFAEVSKLVTDLTKVHTECCHGDL |
| | | LECADDRADLAKYICENQDSISSKLKECCEKPLLEKSHCIAEV |
| | | ENDEMPADLPSLAADFVESKDVCKNYAEAKDVFLGMFLYEY |
| | | ARRHPDYSVVLLLRLAKTYETTLEKCCAAADPHECYAKVFDE |
| | | FKPLVEEPQNLIKQNCELFEQLGEYKFQNALLVRYTKKVPQVS |
| | | TPTLVEVSRNLGKVGSKCCKHPEAKRMPCAEDYLSVVLNQLC |
| | | VLHEKTPVSDRVTKCCTESLVNRRPCFSALEVDETYVPKEFNA |
| | | ETFTFHADICTLSEKERQIKKQTALVELVKHKPKATKEQLKAV |
| | | MDDFAAFVEKCCKADDKETCFAEEGKKLVAASQAALGLGGG |
| | | SAPTSSSTKKTQLQLEHLLLDLQMILNGINNYKNPKLTRMLTF |
| | | KFYMPKKATELKHLQCLEEELKPLEEVLNLAQSKNFHLRPRD |
| | | LISNINVIVLELKGSETTFMCEYADETATIVEFLNRWITFCQSIIS |
| | | TLTGGGS |
| 19 | Albumin | KVQLVESGGGLVQPGGSLRLSCAASGFTFSSFGMTWVRQAPG |
| | binding domain | KGLEWVSSISGSGSDTLYADSVRGRFTISRDNSKNTLYLQMNS |
| | | LRAEDTAVYYCTIGGSLSPSSQGTLVTVSS |
| 20 | HSA domain I | DAHKSEVAHRFKDLGEENFKALVLIAFAQYLQQCPFEDHVKL |
| | | VNEVTEFAKTCVADESAENCDKSLHTLFGDKLCTVATLRETY |
| | | GEMADCCAKQEPERNECFLQHKDDNPNLPRLVRPEVDVMCT |
| | | AFHDNEETFLKKYLYEIARRHPYFYAPELLFFAKRYKAAFTEC |
| | | CQAADKAACLLPKLDELRDEGKASSAKQR |
| 21 | HSA domain II | GKASSAKQRLKCASLQKFGERAFKAWAVARLSQRFPKAEFA |
| | | EVSKLVTDLTKVHTECCHGDLLECADDRADLAKYICENQDSI |
| | | SSKLKECCEKPLLEKSHCIAEVENDEMPADLPSLAADFVESKD |
| | | VCKNYAEAKDVFLGMFLYEYARRHPDYSWLLLRLAKTYETT |
| | | LEKCCAAADPHECYAKVFDEFKPLVEEPQ |
| 22 | HSA domain | NLIKQNCELFEQLGEYKFQNALLVRYTKKVPQVSTPTLVEVSR |
| | III | NLGKVGSKCCKHPEAKRMPCAEDYLSVVLNQLCVLHEKTPV |
| | | SDRVTKCCTESLVNRRPCFSALEVDETYVPKEFNAETFTFHAD |
| | | ICTLSEKERQIKKQTALVELVKHKPKATKEQLKAVMDDFAAF |
| | | VEKCCKADDKETCFAEEGKKLVAASQAALGL |

IV. Linkers

In certain embodiments, the fusion proteins described herein comprise one or more linkers. In certain embodiments, the linker connects one element of the fusion protein to another. In certain embodiments, the linker connects one domain of the fusion protein to another. In certain embodiments, the fusion proteins described herein comprise one, two, three, four, five or more linkers. In some embodiments, the linker is “short,” e.g., consists of 0, 1, 2, 3, 4, 5, 6, 7, 8, 9, 10, 11 or 12 amino acid residues. Thus, in certain instances, the linker consist of about 12 or fewer amino acid residues. In the case of 0 amino acid residues, the linker is a peptide bond. In some embodiments, the linker consists of about 3 to about 50, for example 8, 9 or 10 contiguous amino acid residues. In some embodiments, the linker comprises 0 to about 100 amino acid residues. In some embodiments, the linker comprises about 5 to about 50 amino acid residues. In some embodiments, the linker comprises about 5 to about 15 amino acid residues. In certain embodiments, the linker is a non-peptide linker. In certain embodiments, the linker connects one element of the fusion protein to another via a covalent bond. In certain embodiments, the linker connects one element of the fusion protein to another via a non-covalent bond. In certain embodiments, the fusion proteins described herein comprise more than one type of linker, and/or more than one linker of the same or different lengths (e.g., number of amino acid residues).

### Peptide Linkers

Exemplary linkers include gly-ser polypeptide linkers, glycine-praline polypeptide linkers, and praline-alanine polypeptide linkers. In certain embodiments, the linear polypeptide spacers is a gly-ser polypeptide linker, i.e., a peptide that consists of glycine and serine residues.

In some embodiments, the linker is a peptide linker comprising one or more amino acids, typically about 2-20 amino acids, that are described herein or are known in the art. Suitable, non-immunogenic linker peptides include, for example, (G₄S)ₙ, (SG₄)ₙ or G₄(SG₄)ₙ linker peptides, wherein n is generally a number between 1 and 10, typically between 2 and 4.

Exemplary gly-ser polypeptide linkers comprise the amino acid sequence Ser(Gly₄Ser)ₙ. In certain embodiments, n=1. In certain embodiments, n=2. In certain embodiments, n=3, i.e., Ser(Gly₄Ser)₃. In certain embodiments, n=4, i.e., Ser(Gly₄Ser)₄. In certain embodiments, n=5. In certain embodiments, n=6. In certain embodiments, n=7. In certain embodiments, n=8. In certain embodiments, n=9. In certain embodiments, n=10. Another exemplary gly-ser polypeptide linker comprises the amino acid sequence Ser(Gly₄Ser)ₙ. In certain embodiments, n=1. In certain embodiments, n=2. In certain embodiments, n=3. In certain embodiments, n=4. In certain embodiments, n=5. In certain embodiments, n=6. Another exemplary gly-ser polypeptide linker comprises (Gly₄Ser)ₙ. In certain embodiments, n=1. In certain embodiments, n=2. In certain embodiments, n=3. In certain embodiments, n=4. In certain embodiments, n=5. In certain embodiments, n=6. Another exemplary gly-ser polypeptide linker comprises (Gly₃Ser)ₙ. In certain embodiments, n=1. In certain embodiments, n=2. In certain embodiments, n=3. In certain embodiments, n=4. In certain embodiments, n=5. In certain embodiments n=6.

In some embodiments, the IL-2 is operably linked to a collagen-binding domain by a linker, e.g., a gly-ser linker. In some embodiments, the IL-2 is operably linked to a linear peptide spacer by a linker, e.g., a gly-ser linker. In some embodiments, the IL-12 is operably linked to a collagen-binding domain by a linker, e.g., a gly-ser linker. In some embodiments, the IL-12 is operably linked to a linear polypeptide spacer by a linker, e.g., a gly-ser linker. In some embodiments, the collagen-binding domain is operably linked to a linear polypeptide spacer by a linker, e.g., a gly-ser linker.

V. Exemplary Immunomodulatory Fusion Proteins

The disclosure provides immunomodulatory fusion proteins comprising an immunomodulatory domain and a collagen-binding domain. The immunomodulatory fusion proteins of the disclosure are modular and can be configured to incorporate various individual domains.

### A. IL-2 and IL-12 Fusion Proteins

In some embodiments, the immunomodulatory fusion protein comprises IL-2, IL-12, lumican and a linear polypeptide space, wherein IL-2 is operably linked to lumican. In some embodiments, the immunomodulatory fusion protein comprises IL-2, IL-12, lumican and a linear polypeptide space, wherein IL-2 is operably linked to the linear polypeptide spacer. In some embodiments, the immunomodulatory fusion protein comprises IL-2, IL-12, lumican, and a linear polypeptide spacer wherein IL-12 is operably linked to lumican. In some embodiments, the immunomodulatory fusion protein comprises IL-2, IL-12, lumican, and a linear polypeptide spacer wherein IL-12 is operably linked to the linear polypeptide spacer.

In some embodiments, the immunomodulatory fusion protein comprises IL-2, IL-12, LAIR1 and a linear polypeptide space, wherein IL-2 is operably linked to LAIR1. In some embodiments, the immunomodulatory fusion protein comprises IL-2, IL-12, LAIR1 and a linear polypeptide space, wherein IL-2 is operably linked to the linear polypeptide spacer. In some embodiments, the immunomodulatory fusion protein comprises IL-2, IL-12, LAIR1, and a linear polypeptide spacer wherein IL-12 is operably linked to LAIR1. In some embodiments, the immunomodulatory fusion protein comprises IL-2, IL-12, LAIR1, and a linear polypeptide spacer wherein IL-12 is operably linked to the linear polypeptide spacer.

In some embodiments, the immunomodulatory fusion protein comprises IL-2, IL-12, LAIR2 and a linear polypeptide space, wherein IL-2 is operably linked to LAIR2. In some embodiments, the immunomodulatory fusion protein comprises IL-2, IL-12, LAIR2 and a linear polypeptide space, wherein IL-2 is operably linked to the linear polypeptide spacer. In some embodiments, the immunomodulatory fusion protein comprises IL-2, IL-12, LAIR2, and a linear polypeptide spacer wherein IL-12 is operably linked to LAIR2. In some embodiments, the immunomodulatory fusion protein comprises IL-2, IL-12, LAIR2, and a linear polypeptide spacer wherein IL-12 is operably linked to the linear polypeptide spacer.

In some embodiments, the immunomodulatory fusion protein comprises an amino acid sequence having at least 80%, at least 81%, at least 82%, at least 83%, at least 84%, at least 85%, at least 86, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence as set forth in SEQ ID NOs: 23-70, or a portion thereof.

In some embodiments, the immunomodulatory fusion protein comprises an amino acid sequence having a leader sequence as set forth in SEQ ID NO: 71: MRVPAQLLGLLLLWLPGARCA.

In some embodiments, the immunomodulatory fusion protein comprises an amino acid sequence having a His tag sequence as set forth in SEQ ID NO: 72: HEIHHHEIHHHH.

In some embodiments, the immunomodulatory fusion protein comprises an amino acid sequence having at least 80% identity to the amino acid sequence set forth in SEQ ID NOs: 23-70 or a portion thereof, wherein the immunomodulatory fusion protein excludes the leader sequence of SEQ ID NO: 71: MRVPAQLLGLLLLWLPGARCA.

In some embodiments, the immunomodulatory fusion protein comprises an amino acid sequence having at least 80% identity to the amino acid sequence set forth in SEQ ID NOs: 23-70 or a portion thereof, wherein the immunomodulatory fusion protein excludes the His tag sequence of SEQ ID NO: 72: HHHHHHHHHH.

In some embodiments, the immunomodulatory fusion protein comprises an amino acid sequence having at least 80% identity to the amino acid sequence set forth in SEQ ID NOs: 23-70 or a portion thereof, wherein the immunomodulatory fusion protein excludes the leader sequence of SEQ ID NO: 71: MRVPAQLLGLLLLWLPGARCA. and the His tag sequence of SEQ ID NO: 72: HEIREIHHHHHH.

In some embodiments, the immunomodulatory fusion protein comprises an amino acid sequence having at least 80% identity to a portion of the amino acid sequence set forth in SEQ ID NOs: 23-70, wherein the portion excludes the leader sequence having an amino acid sequence set forth in SEQ ID NO: 71.

In some embodiments, the immunomodulatory fusion protein comprises an amino acid sequence having at least 80% identity to a portion of the amino acid sequence set forth in SEQ ID NOs: 23-70, wherein the portion excludes the His tag sequence having an amino acid sequence set forth in SEQ ID NO: 72.

In some embodiments, the immunomodulatory fusion protein comprises an amino acid sequence having at least 80% identity to a portion of the amino acid sequence set forth in SEQ ID NOs: 23-70, wherein the portion excludes the leader sequence having an amino acid sequence set forth in SEQ ID NO: 71 and the portion further excludes the His tag sequence having an amino acid sequence set forth in SEQ ID NO: 72.

In some embodiments, the immunomodulatory fusion protein comprises an amino acid sequence having at least 80% identity to a portion of the amino acid sequence set forth in SEQ ID NO: 73. In some embodiments, the immunomodulatory fusion protein comprises an amino acid sequence having at least 85%, at least 90%, at least 91%, at least 92%, at least 93%, at least 94%, at least 95%, at least 96%, at least 97%, at least 98%, at least 99%, or 100% identity to the amino acid sequence set forth in SEQ ID NO: 73.

TABLE 5
Exemplary murine bi-functional linear constructs

| SEQ ID | Construct | |
|---|---|---|
| NO: | Name | Sequence |
| 23 | 12-MSA- | MWELEKDVYVVEVDWTPDAPGETVNLTCDTPEEDDITWTSDQ |
| | Lum-MSA-2 | RHGVIGSGKTLTITVKEFLDAGQYTCHKGGETLSHSHLLLHKK |
| | | ENGIWSTEILKNFKNKTFLKCEAPNYSGRFTCSWLVQRNMDLK |
| | | FNIKSSSSSPDSRAVTCGMASLSAEKVTLDQRDYEKYSVSCQE |
| | | DVTCPTAEETLPIELALEARQQNKYENYSTSFFIRDIIKPDPPKN |
| | | LQMKPLKNSQVEVSWEYPDSWSTPHSYFSLKFFVRIQRKKEK |
| | | MKETEEGCNQKGAFLVEKTSTEVQCKGGNVCVQAQDRYYNS |
| | | SCSKWACVPCRVRSGGSGGGSGGGSGGGSRVIPVSGPARCLSQ |
| | | SRNLLKTTDDMVKTAREKLKHYSCTAEDIDHEDITRDQTSTLK |
| | | TCLPLELHKNESCLATRETSSTTRGSCLPPQKTSLMMTLCLGSI |
| | | YEDLKMYQTEFQAINAALQNHNHQQIILDKGMLVAIDELMQS |
| | | LNHNGETLRQKPPVGEADPYRVKMKLCILLHAFSTRVVTINRV |
| | | MGYLSSAGSGGGSEAHKSEIAHRYNDLGEQHFKGLVLIAFSQY |
| | | LQKCSYDEHAKLVQEVTDFAKTCVADESAANCDKSLHTLFGD |
| | | KLCAIPNLRENYGELADCCTKQEPERNECFLQHKDDNPSLPPFE |
| | | RPEAEAMCTSFKENPTTFMGHYLHEVARRHPYFYAPELLYYAE |
| | | QYNEILTQCCAEADKESCLTPKLDGVKEKALVSSVRQRMKCSS |
| | | MQKFGERAFKAWAVARLSQTFPNADFAEITKLATDLTKVNKE |
| | | CCHGDLLECADDRAELAKYMCENQATISSKLQTCCDKPLLKK |
| | | AHCLSEVEHDTMPADLPAIAADFVEDQEVCKNYAEAKDVFLG |
| | | TFLYEYSRRHPDYSVSLLLRLAKKYEATLEKCCAEANPPACYG |
| | | TVLAEFQPLVEEPKNLVKTNCDLYEKLGEYGFQNAILVRYTQK |
| | | APQVSTPTLVEAARNLGRVGTKCCTLPEDQRLPCVEDYLSAIL |
| | | NRVCLLHEKTPVSEHVTKCCSGSLVERRPCFSALTVDETYVPK |
| | | EFKAETFTFHSDICTLPEKEKQIKKQTALAELVKHKPKATAEQL |
| | | KTVMDDFAQFLDTCCKAADKDTCFSTEGPNLVTRCKDALAGG |
| | | GSGGGSSQYYDYDIPLFMYGQISPNCAPECNCPHSYPTAMYCD |
| | | DLKLKSVPMVPPGIKYLYLRNNQIDHIDEKAFENVTDLQWLIL |
| | | DHNLLENSKIKGKVFSKLKQLKKLHINYNNLTESVGPLPKSLQ |
| | | DLQLTNNKISKLGSFDGLVNLTFIYLQHNQLKEDAVSASLKGL |
| | | KSLEYLDLSFNQMSKLPAGLPTSLLTLYLDNNKISNIPDEYFKR |
| | | FTGLQYLRLSHNELADSGVPGNSFNISSLLELDLSYNKLKSIPTV |
| | | NENLENYYLEVNELEKFDVKSFCKILGPLSYSKIKHLRLDGNPL |
| | | TQSSLPPDMYECLRVANEITVNGGGSGGGSEAHKSEIAHRYND |
| | | LGEQHFKGLVLIAFSQYLQKCSYDEHAKLVQEVTDFAKTCVA |
| | | DESAANCDKSLHTLFGDKLCAIPNLRENYGELADCCTKQEPER |
| | | NECFLQHKDDNPSLPPFERPEAEAMCTSFKENPTTFMGHYLHE |
| | | VARRHPYFYAPELLYYAEQYNEILTQCCAEADKESCLTPKLDG |
| | | VKEKALVSSVRQRMKCSSMQKFGERAFKAWAVARLSQTFPN |
| | | ADFAEITKLATDLTKVNKECCHGDLLECADDRAELAKYMCEN |
| | | QATISSKLQTCCDKPLLKKAHCLSEVEHDTMPADLPAIAADFV |
| | | EDQEVCKNYAEAKDVFLGTFLYEYSRRHPDYSVSLLLRLAKK |
| | | YEATLEKCCAEANPPACYGTVLAEFQPLVEEPKNLVKTNCDLY |
| | | EKLGEYGFQNAILVRYTQKAPQVSTPTLVEAARNLGRVGTKCC |
| | | TLPEDQRLPCVEDYLSAILNRVCLLHEKTPVSEHVTKCCSGSLV |
| | | ERRPCFSALTVDETYVPKEFKAETFTFHSDICTLPEKEKQIKKQT |
| | | ALAELVKHKPKATAEQLKTVMDDFAQFLDTCCKAADKDTCFS |
| | | TEGPNLVTRCKDALAGGGSAPTSSSTSSSTAEAQQQQQQQQQQ |
| | | QQHLEQLLMDLQELLSRMENYRNLKLPRMLTFKFYLPKQATE |
| | | LKDLQCLEDELGPLRHVLDLTQSKSFQLEDAENFISNIRVTVVK |
| | | LKGSDNTFECQFDDESATVVDFLRRWIAFCQSIISTSPQHHHHH |
| | | HHHHHH |
| 24 | 12-Lum- | MWELEKDVYVVEVDWTPDAPGETVNLTCDTPEEDDITWTSDQ |
| | MSA-2 | RHGVIGSGKTLTITVKEFLDAGQYTCHKGGETLSHSHLLLHKK |
| | | ENGIWSTEILKNFKNKTFLKCEAPNYSGRFTCSWLVQRNMDLK |
| | | FNIKSSSSSPDSRAVTCGMASLSAEKVTLDQRDYEKYSVSCQE |
| | | DVTCPTAEETLPIELALEARQQNKYENYSTSFFIRDIIKPDPPKN |
| | | LQMKPLKNSQVEVSWEYPDSWSTPHSYFSLKFFVRIQRKKEK |
| | | MKETEEGCNQKGAFLVEKTSTEVQCKGGNVCVQAQDRYYNS |
| | | SCSKWACVPCRVRSGGSGGGSGGGSGGGSRVIPVSGPARCLSQ |
| | | SRNLLKTTDDMVKTAREKLKHYSCTAEDIDHEDITRDQTSTLK |
| | | TCLPLELHKNESCLATRETSSTTRGSCLPPQKTSLMMTLCLGSI |
| | | YEDLKMYQTEFQAINAALQNHNHQQIILDKGMLVAIDELMQS |
| | | LNHNGETLRQKPPVGEADPYRVKMKLCILLHAFSTRVVTINRV |
| | | MGYLSSAGGGSGGGSSQYYDYDIPLFMYGQISPNCAPECNCPH |
| | | SYPTAMYCDDLKLKSVPMVPPGIKYLYLRNNQIDHIDEKAFEN |
| | | VTDLQWLILDHNLLENSKIKGKVFSKLKQLKKLHINYNNLTES |
| | | VGPLPKSLQDLQLTNNKISKLGSFDGLVNLTFIYLQHNQLKEDA |
| | | VSASLKGLKSLEYLDLSFNQMSKLPAGLPTSLLTLYLDNNKISN |
| | | IPDEYFKRFTGLQYLRLSHNELADSGVPGNSFNISSLLELDLSYN |
| | | KLKSIPTVNENLENYYLEVNELEKFDVKSFCKILGPLSYSKIKHL |
| | | RLDGNPLTQSSLPPDMYECLRVANEITVNGGGSGGGSEAHKSE |
| | | IAHRYNDLGEQHFKGLVLIAFSQYLQKCSYDEHAKLVQEVTDF |
| | | AKTCVADESAANCDKSLHTLFGDKLCAIPNLRENYGELADCCT |
| | | KQEPERNECFLQHKDDNPSLPPFERPEAEAMCTSFKENPTTFM |
| | | GHYLHEVARRHPYFYAPELLYYAEQYNEILTQCCAEADKESCL |
| | | TPKLDGVKEKALVSSVRQRMKCSSMQKFGERAFKAWAVARL |
| | | SQTFPNADFAEITKLATDLTKVNKECCHGDLLECADDRAELAK |
| | | YMCENQATISSKLQTCCDKPLLKKAHCLSEVEHDTMPADLPAI |
| | | AADFVEDQEVCKNYAEAKDVFLGTFLYEYSRRHPDYSVSLLL |
| | | RLAKKYEATLEKCCAEANPPACYGTVLAEFQPLVEEPKNLVKT |
| | | NCDLYEKLGEYGFQNAILVRYTQKAPQVSTPTLVEAARNLGR |
| | | VGTKCCTLPEDQRLPCVEDYLSAILNRVCLLHEKTPVSEHVTK |
| | | CCSGSLVERRPCFSALTVDETYVPKEFKAETFTFHSDICTLPEKE |
| | | KQIKKQTALAELVKHKPKATAEQLKTVMDDFAQFLDTCCKAA |
| | | DKDTCFSTEGPNLVTRCKDALAGGGSAPTSSSTSSSTAEAQQQ |
| | | QQQQQQQQQHLEQLLMDLQELLSRMENYRNLKLPRMLTFKF |
| | | YLPKQATELKDLQCLEDELGPLRHVLDLTQSKSFQLEDAENFIS |
| | | NIRVTVVKLKGSDNTFECQFDDESATVVDFLRRWIAFCQSIISTS |
| | | PQHHHHHHHHHH |
| 25 | 12-MSA- | MWELEKDVYVVEVDWTPDAPGETVNLTCDTPEEDDITWTSDQ |
| | Lum-2 | RHGVIGSGKTLTITVKEFLDAGQYTCHKGGETLSHSHLLLHKK |
| | | ENGIWSTEILKNFKNKTFLKCEAPNYSGRFTCSWLVQRNMDLK |
| | | FNIKSSSSSPDSRAVTCGMASLSAEKVTLDQRDYEKYSVSCQE |
| | | DVTCPTAEETLPIELALEARQQNKYENYSTSFFIRDIIKPDPPKN |
| | | LQMKPLKNSQVEVSWEYPDSWSTPHSYFSLKFFVRIQRKKEK |
| | | MKETEEGCNQKGAFLVEKTSTEVQCKGGNVCVQAQDRYYNS |
| | | SCSKWACVPCRVRSGGSGGGSGGGSGGGSRVIPVSGPARCLSQ |
| | | SRNLLKTTDDMVKTAREKLKHYSCTAEDIDHEDITRDQTSTLK |
| | | TCLPLELHKNESCLATRETSSTTRGSCLPPQKTSLMMTLCLGSI |
| | | YEDLKMYQTEFQAINAALQNHNHQQIILDKGMLVAIDELMQS |
| | | LNHNGETLRQKPPVGEADPYRVKMKLCILLHAFSTRVVTINRV |
| | | MGYLSSAGSGGGSEAHKSEIAHRYNDLGEQHFKGLVLIAFSQY |
| | | LQKCSYDEHAKLVQEVTDFAKTCVADESAANCDKSLHTLFGD |
| | | KLCAIPNLRENYGELADCCTKQEPERNECFLQHKDDNPSLPPFE |
| | | RPEAEAMCTSFKENPTTFMGHYLHEVARRHPYFYAPELLYYAE |
| | | QYNEILTQCCAEADKESCLTPKLDGVKEKALVSSVRQRMKCSS |
| | | MQKFGERAFKAWAVARLSQTFPNADFAEITKLATDLTKVNKE |
| | | CCHGDLLECADDRAELAKYMCENQATISSKLQTCCDKPLLKK |
| | | AHCLSEVEHDTMPADLPAIAADFVEDQEVCKNYAEAKDVFLG |
| | | TFLYEYSRRHPDYSVSLLLRLAKKYEATLEKCCAEANPPACYG |
| | | TVLAEFQPLVEEPKNLVKTNCDLYEKLGEYGFQNAILVRYTQK |
| | | APQVSTPTLVEAARNLGRVGTKCCTLPEDQRLPCVEDYLSAIL |
| | | NRVCLLHEKTPVSEHVTKCCSGSLVERRPCFSALTVDETYVPK |
| | | EFKAETFTFHSDICTLPEKEKQIKKQTALAELVKHKPKATAEQL |
| | | KTVMDDFAQFLDTCCKAADKDTCFSTEGPNLVTRCKDALAGG |
| | | GSGGGSSQYYDYDIPLFMYGQISPNCAPECNCPHSYPTAMYCD |
| | | DLKLKSVPMVPPGIKYLYLRNNQIDHIDEKAFENVTDLQWLIL |
| | | DHNLLENSKIKGKVFSKLKQLKKLHINYNNLTESVGPLPKSLQ |
| | | DLQLTNNKISKLGSFDGLVNLTFIYLQHNQLKEDAVSASLKGL |
| | | KSLEYLDLSFNQMSKLPAGLPTSLLTLYLDNNKISNIPDEYFKR |
| | | FTGLQYLRLSHNELADSGVPGNSFNISSLLELDLSYNKLKSIPTV |
| | | NENLENYYLEVNELEKFDVKSFCKILGPLSYSKIKHLRLDGNPL |
| | | TQSSLPPDMYECLRVANEITVNGGGSGGGSAPTSSSTSSSTAEA |
| | | QQQQQQQQQQQQHLEQLLMDLQELLSRMENYRNLKLPRMLT |
| | | FKFYLPKQATELKDLQCLEDELGPLRHVLDLTQSKSFQLEDAE |
| | | NFISNIRVTVVKLKGSDNTFECQFDDESATVVDFLRRWIAFCQS |
| | | IISTSPQHHHHHHHHHH |
| 26 | 12-MSA- | MWELEKDVYVVEVDWTPDAPGETVNLTCDTPEEDDITWTSDQ |
| | LAIR-MSA-2 | RHGVIGSGKTLTITVKEFLDAGQYTCHKGGETLSHSHLLLHKK |
| | | ENGIWSTEILKNFKNKTFLKCEAPNYSGRFTCSWLVQRNMDLK |
| | | FNIKSSSSSPDSRAVTCGMASLSAEKVTLDQRDYEKYSVSCQE |
| | | DVTCPTAEETLPIELALEARQQNKYENYSTSFFIRDIIKPDPPKN |
| | | LQMKPLKNSQVEVSWEYPDSWSTPHSYFSLKFFVRIQRKKEK |
| | | MKETEEGCNQKGAFLVEKTSTEVQCKGGNVCVQAQDRYYNS |
| | | SCSKWACVPCRVRSGGSGGGSGGGSGGGSRVIPVSGPARCLSQ |
| | | SRNLLKTTDDMVKTAREKLKHYSCTAEDIDHEDITRDQTSTLK |
| | | TCLPLELHKNESCLATRETSSTTRGSCLPPQKTSLMMTLCLGSI |
| | | YEDLKMYQTEFQAINAALQNHNHQQIILDKGMLVAIDELMQS |
| | | LNHNGETLRQKPPVGEADPYRVKMKLCILLHAFSTRVVTINRV |
| | | MGYLSSAGSGGGSEAHKSEIAHRYNDLGEQHFKGLVLIAFSQY |
| | | LQKCSYDEHAKLVQEVTDFAKTCVADESAANCDKSLHTLFGD |
| | | KLCAIPNLRENYGELADCCTKQEPERNECFLQHKDDNPSLPPFE |
| | | RPEAEAMCTSFKENPTTFMGHYLHEVARRHPYFYAPELLYYAE |
| | | QYNEILTQCCAEADKESCLTPKLDGVKEKALVSSVRQRMKCSS |
| | | MQKFGERAFKAWAVARLSQTFPNADFAEITKLATDLTKVNKE |
| | | CCHGDLLECADDRAELAKYMCENQATISSKLQTCCDKPLLKK |
| | | AHCLSEVEHDTMPADLPAIAADFVEDQEVCKNYAEAKDVFLG |
| | | TFLYEYSRRHPDYSVSLLLRLAKKYEATLEKCCAEANPPACYG |
| | | TVLAEFQPLVEEPKNLVKTNCDLYEKLGEYGFQNAILVRYTQK |
| | | APQVSTPTLVEAARNLGRVGTKCCTLPEDQRLPCVEDYLSAIL |
| | | NRVCLLHEKTPVSEHVTKCCSGSLVERRPCFSALTVDETYVPK |
| | | EFKAETFTFHSDICTLPEKEKQIKKQTALAELVKHKPKATAEQL |
| | | KTVMDDFAQFLDTCCKAADKDTCFSTEGPNLVTRCKDALAGG |
| | | GSGGGSQEGSLPDITIFPNSSLMISQGTFVTVVCSYSDKHDLYN |
| | | MVRLEKDGSTFMEKSTEPYKTEDEFEIGPVNETITGHYSCIYSK |
| | | GITWSERSKTLELKVIKENVIQTPAPGPTSDTSWLKTYSIYGGGS |
| | | GGGSEAHKSEIAHRYNDLGEQHFKGLVLIAFSQYLQKCSYDEH |
| | | AKLVQEVTDFAKTCVADESAANCDKSLHTLFGDKLCAIPNLRE |
| | | NYGELADCCTKQEPERNECFLQHKDDNPSLPPFERPEAEAMCT |
| | | SFKENPTTFMGHYLHEVARRHPYFYAPELLYYAEQYNEILTQC |
| | | CAEADKESCLTPKLDGVKEKALVSSVRQRMKCSSMQKFGERA |
| | | FKAWAVARLSQTFPNADFAEITKLATDLTKVNKECCHGDLLEC |
| | | ADDRAELAKYMCENQATISSKLQTCCDKPLLKKAHCLSEVEH |
| | | DTMPADLPAIAADFVEDQEVCKNYAEAKDVFLGTFLYEYSRR |
| | | HPDYSVSLLLRLAKKYEATLEKCCAEANPPACYGTVLAEFQPL |
| | | VEEPKNLVKTNCDLYEKLGEYGFQNAILVRYTQKAPQVSTPTL |
| | | VEAARNLGRVGTKCCTLPEDQRLPCVEDYLSAILNRVCLLHEK |
| | | TPVSEHVTKCCSGSLVERRPCFSALTVDETYVPKEFKAETFTFH |
| | | SDICTLPEKEKQIKKQTALAELVKHKPKATAEQLKTVMDDFAQ |
| | | FLDTCCKAADKDTCFSTEGPNLVTRCKDALAGGGSAPTSSSTS |
| | | SSTAEAQQQQQQQQQQQQHLEQLLMDLQELLSRMENYRNLK |
| | | LPRMLTFKFYLPKQATELKDLQCLEDELGPLRHVLDLTQSKSF |
| | | QLEDAENFISNIRVTVVKLKGSDNTFECQFDDESATVVDFLRR |
| | | WIAFCQSIISTSPQHHHHHHHHHH |
| 27 | 12-LAIR- | MWELEKDVYVVEVDWTPDAPGETVNLTCDTPEEDDITWTSDQ |
| | MSA-2 | RHGVIGSGKTLTITVKEFLDAGQYTCHKGGETLSHSHLLLHKK |
| | | ENGIWSTEILKNFKNKTFLKCEAPNYSGRFTCSWLVQRNMDLK |
| | | FNIKSSSSSPDSRAVTCGMASLSAEKVTLDQRDYEKYSVSCQE |
| | | DVTCPTAEETLPIELALEARQQNKYENYSTSFFIRDIIKPDPPKN |
| | | LQMKPLKNSQVEVSWEYPDSWSTPHSYFSLKFFVRIQRKKEK |
| | | MKETEEGCNQKGAFLVEKTSTEVQCKGGNVCVQAQDRYYNS |
| | | SCSKWACVPCRVRSGGSGGGSGGGSGGGSRVIPVSGPARCLSQ |
| | | SRNLLKTTDDMVKTAREKLKHYSCTAEDIDHEDITRDQTSTLK |
| | | TCLPLELHKNESCLATRETSSTTRGSCLPPQKTSLMMTLCLGSI |
| | | YEDLKMYQTEFQAINAALQNHNHQQIILDKGMLVAIDELMQS |
| | | LNHNGETLRQKPPVGEADPYRVKMKLCILLHAFSTRVVTINRV |
| | | MGYLSSAGGGSGGGSQEGSLPDITIFPNSSLMISQGTFVTVVCS |
| | | YSDKHDLYNMVRLEKDGSTFMEKSTEPYKTEDEFEIGPVNETI |
| | | TGHYSCIYSKGITWSERSKTLELKVIKENVIQTPAPGPTSDTSWL |
| | | KTYSIYGGGSGGGSEAHKSEIAHRYNDLGEQHFKGLVLIAFSQ |
| | | YLQKCSYDEHAKLVQEVTDFAKTCVADESAANCDKSLHTLFG |
| | | DKLCAIPNLRENYGELADCCTKQEPERNECFLQHKDDNPSLPPF |
| | | ERPEAEAMCTSFKENPTTFMGHYLHEVARRHPYFYAPELLYYA |
| | | EQYNEILTQCCAEADKESCLTPKLDGVKEKALVSSVRQRMKCS |
| | | SMQKFGERAFKAWAVARLSQTFPNADFAEITKLATDLTKVNK |
| | | ECCHGDLLECADDRAELAKYMCENQATISSKLQTCCDKPLLK |
| | | KAHCLSEVEHDTMPADLPAIAADFVEDQEVCKNYAEAKDVFL |
| | | GTFLYEYSRRHPDYSVSLLLRLAKKYEATLEKCCAEANPPACY |
| | | GTVLAEFQPLVEEPKNLVKTNCDLYEKLGEYGFQNAILVRYTQ |
| | | KAPQVSTPTLVEAARNLGRVGTKCCTLPEDQRLPCVEDYLSAI |
| | | LNRVCLLHEKTPVSEHVTKCCSGSLVERRPCFSALTVDETYVP |
| | | KEFKAETFTFHSDICTLPEKEKQIKKQTALAELVKHKPKATAEQ |
| | | LKTVMDDFAQFLDTCCKAADKDTCFSTEGPNLVTRCKDALAG |
| | | GGSAPTSSSTSSSTAEAQQQQQQQQQQQQHLEQLLMDLQELLS |
| | | RMENYRNLKLPRMLTFKFYLPKQATELKDLQCLEDELGPLRH |
| | | VLDLTQSKSFQLEDAENFISNIRVTVVKLKGSDNTFECQFDDES |
| | | ATVVDFLRRWIAFCQSIISTSPQHHHHHHHHHH |
| 28 | 12-MSA- | MWELEKDVYVVEVDWTPDAPGETVNLTCDTPEEDDITWTSDQ |
| | LAIR-2 | RHGVIGSGKTLTITVKEFLDAGQYTCHKGGETLSHSHLLLHKK |
| | | ENGIWSTEILKNFKNKTFLKCEAPNYSGRFTCSWLVQRNMDLK |
| | | FNIKSSSSSPDSRAVTCGMASLSAEKVTLDQRDYEKYSVSCQE |
| | | DVTCPTAEETLPIELALEARQQNKYENYSTSFFIRDIIKPDPPKN |
| | | LQMKPLKNSQVEVSWEYPDSWSTPHSYFSLKFFVRIQRKKEK |
| | | MKETEEGCNQKGAFLVEKTSTEVQCKGGNVCVQAQDRYYNS |
| | | SCSKWACVPCRVRSGGSGGGSGGGSGGGSRVIPVSGPARCLSQ |
| | | SRNLLKTTDDMVKTAREKLKHYSCTAEDIDHEDITRDQTSTLK |
| | | TCLPLELHKNESCLATRETSSTTRGSCLPPQKTSLMMTLCLGSI |
| | | YEDLKMYQTEFQAINAALQNHNHQQIILDKGMLVAIDELMQS |
| | | LNHNGETLRQKPPVGEADPYRVKMKLCILLHAFSTRVVTINRV |
| | | MGYLSSAGSGGGSEAHKSEIAHRYNDLGEQHFKGLVLIAFSQY |
| | | LQKCSYDEHAKLVQEVTDFAKTCVADESAANCDKSLHTLFGD |
| | | KLCAIPNLRENYGELADCCTKQEPERNECFLQHKDDNPSLPPFE |
| | | RPEAEAMCTSFKENPTTFMGHYLHEVARRHPYFYAPELLYYAE |
| | | QYNEILTQCCAEADKESCLTPKLDGVKEKALVSSVRQRMKCSS |
| | | MQKFGERAFKAWAVARLSQTFPNADFAEITKLATDLTKVNKE |
| | | CCHGDLLECADDRAELAKYMCENQATISSKLQTCCDKPLLKK |
| | | AHCLSEVEHDTMPADLPAIAADFVEDQEVCKNYAEAKDVFLG |
| | | TFLYEYSRRHPDYSVSLLLRLAKKYEATLEKCCAEANPPACYG |
| | | TVLAEFQPLVEEPKNLVKTNCDLYEKLGEYGFQNAILVRYTQK |
| | | APQVSTPTLVEAARNLGRVGTKCCTLPEDQRLPCVEDYLSAIL |
| | | NRVCLLHEKTPVSEHVTKCCSGSLVERRPCFSALTVDETYVPK |
| | | EFKAETFTFHSDICTLPEKEKQIKKQTALAELVKHKPKATAEQL |
| | | KTVMDDFAQFLDTCCKAADKDTCFSTEGPNLVTRCKDALAGG |
| | | GSGGGSQEGSLPDITIFPNSSLMISQGTFVTVVCSYSDKHDLYN |
| | | MVRLEKDGSTFMEKSTEPYKTEDEFEIGPVNETITGHYSCIYSK |
| | | GITWSERSKTLELKVIKENVIQTPAPGPTSDTSWLKTYSIYGGGS |
| | | GGGSAPTSSSTSSSTAEAQQQQQQQQQQQQHLEQLLMDLQEL |
| | | LSRMENYRNLKLPRMLTFKFYLPKQATELKDLQCLEDELGPLR |
| | | HVLDLTQSKSFQLEDAENFISNIRVTVVKLKGSDNTFECQFDDE |
| | | SATVVDFLRRWIAFCQSIISTSPQHHHHHHHHHH |
| 29 | 12-Lum- | MRVPAQLLGLLLLWLPGARCAMWELEKDVYVVEVDWTPDAP |
| | ABD-2 | GETVNLTCDTPEEDDITWTSDQRHGVIGSGKTLTITVKEFLDAG |
| | | QYTCHKGGETLSHSHLLLHKKENGIWSTEILKNFKNKTFLKCE |
| | | APNYSGRFTCSWLVQRNMDLKFNIKSSSSSPDSRAVTCGMASL |
| | | SAEKVTLDQRDYEKYSVSCQEDVTCPTAEETLPIELALEARQQ |
| | | NKYENYSTSFFIRDIIKPDPPKNLQMKPLKNSQVEVSWEYPDSW |
| | | STPHSYFSLKFFVRIQRKKEKMKETEEGCNQKGAFLVEKTSTE |
| | | VQCKGGNVCVQAQDRYYNSSCSKWACVPCRVRSGGSGGGSG |
| | | GGSGGGSRVIPVSGPARCLSQSRNLLKTTDDMVKTAREKLKHY |
| | | SCTAEDIDHEDITRDQTSTLKTCLPLELHKNESCLATRETSSTTR |
| | | GSCLPPQKTSLMMTLCLGSIYEDLKMYQTEFQAINAALQNHNH |
| | | QQIILDKGMLVAIDELMQSLNHNGETLRQKPPVGEADPYRVK |
| | | MKLCILLHAFSTRVVTINRVMGYLSSAGGGSGGGSSQYYDYDI |
| | | PLFMYGQISPNCAPECNCPHSYPTAMYCDDLKLKSVPMVPPGI |
| | | KYLYLRNNQIDHIDEKAFENVTDLQWLILDHNLLENSKIKGKV |
| | | FSKLKQLKKLHINYNNLTESVGPLPKSLQDLQLTNNKISKLGSF |
| | | DGLVNLTFIYLQHNQLKEDAVSASLKGLKSLEYLDLSFNQMSK |
| | | LPAGLPTSLLTLYLDNNKISNIPDEYFKRFTGLQYLRLSHNELA |
| | | DSGVPGNSFNISSLLELDLSYNKLKSIPTVNENLENYYLEVNEL |
| | | EKFDVKSFCKILGPLSYSKIKHLRLDGNPLTQSSLPPDMYECLR |
| | | VANEITVNGGGSGGGSKVQLVESGGGLVQPGGSLRLSCAASGF |
| | | TFSSFGMTWVRQAPGKGLEWVSSISGSGSDTLYADSVRGRFTIS |
| | | RDNSKNTLYLQMNSLRAEDTAVYYCTIGGSLSPSSQGTLVTVS |
| | | SGGGSAPTSSSTSSSTAEAQQQQQQQQQQQQHLEQLLMDLQEL |
| | | LSRMENYRNLKLPRMLTFKFYLPKQATELKDLQCLEDELGPLR |
| | | HVLDLTQSKSFQLEDAENFISNIRVTVVKLKGSDNTFECQFDDE |
| | | SATVVDFLRRWIAFCQSIISTSPQHHHHHHHHHH |
| 30 | 12-LAIR- | MRVPAQLLGLLLLWLPGARCAMWELEKDVYVVEVDWTPDAP |
| | ABD2 | GETVNLTCDTPEEDDITWTSDQRHGVIGSGKTLTITVKEFLDAG |
| | | QYTCHKGGETLSHSHLLLHKKENGIWSTEILKNFKNKTFLKCE |
| | | APNYSGRFTCSWLVQRNMDLKFNIKSSSSSPDSRAVTCGMASL |
| | | SAEKVTLDQRDYEKYSVSCQEDVTCPTAEETLPIELALEARQQ |
| | | NKYENYSTSFFIRDIIKPDPPKNLQMKPLKNSQVEVSWEYPDSW |
| | | STPHSYFSLKFFVRIQRKKEKMKETEEGCNQKGAFLVEKTSTE |
| | | VQCKGGNVCVQAQDRYYNSSCSKWACVPCRVRSGGSGGGSG |
| | | GGSGGGSRVIPVSGPARCLSQSRNLLKTTDDMVKTAREKLKHY |
| | | SCTAEDIDHEDITRDQTSTLKTCLPLELHKNESCLATRETSSTTR |
| | | GSCLPPQKTSLMMTLCLGSIYEDLKMYQTEFQAINAALQNHNH |
| | | QQIILDKGMLVAIDELMQSLNHNGETLRQKPPVGEADPYRVK |
| | | MKLCILLHAFSTRVVTINRVMGYLSSAGGGSGGGSQEGSLPDIT |
| | | IFPNSSLMISQGTFVTVVCSYSDKHDLYNMVRLEKDGSTFMEK |
| | | STEPYKTEDEFEIGPVNETITGHYSCIYSKGITWSERSKTLELKVI |
| | | KENVIQTPAPGPTSDTSWLKTYSIYGGGSGGGSKVQLVESGGG |
| | | LVQPGGSLRLSCAASGFTFSSFGMTWVRQAPGKGLEWVSSISG |
| | | SGSDTLYADSVRGRFTISRDNSKNTLYLQMNSLRAEDTAVYYC |
| | | TIGGSLSPSSQGTLVTVSSGGGSAPTSSSTSSSTAEAQQQQQQQ |
| | | QQQQQHLEQLLMDLQELLSRMENYRNLKLPRMLTFKFYLPKQ |
| | | ATELKDLQCLEDELGPLRHVLDLTQSKSFQLEDAENFISNIRVT |
| | | VVKLKGSDNTFECQFDDESATVVDFLRRWIAFCQSIISTSPQHH |
| | | HHHHHHHH |
| 31 | 12-Lum- | MRVPAQLLGLLLLWLPGARCAMWELEKDVYVVEVDWTPDAP |
| | MSA H464Q- | GETVNLTCDTPEEDDITWTSDQRHGVIGSGKTLTITVKEFLDAG |
| | 2 | QYTCHKGGETLSHSHLLLHKKENGIWSTEILKNFKNKTFLKCE |
| | | APNYSGRFTCSWLVQRNMDLKFNIKSSSSSPDSRAVTCGMASL |
| | | SAEKVTLDQRDYEKYSVSCQEDVTCPTAEETLPIELALEARQQ |
| | | NKYENYSTSFFIRDIIKPDPPKNLQMKPLKNSQVEVSWEYPDSW |
| | | STPHSYFSLKFFVRIQRKKEKMKETEEGCNQKGAFLVEKTSTE |
| | | VQCKGGNVCVQAQDRYYNSSCSKWACVPCRVRSGGSGGGSG |
| | | GGSGGGSRVIPVSGPARCLSQSRNLLKTTDDMVKTAREKLKHY |
| | | SCTAEDIDHEDITRDQTSTLKTCLPLELHKNESCLATRETSSTTR |
| | | GSCLPPQKTSLMMTLCLGSIYEDLKMYQTEFQAINAALQNHNH |
| | | QQIILDKGMLVAIDELMQSLNHNGETLRQKPPVGEADPYRVK |
| | | MKLCILLHAFSTRVVTINRVMGYLSSAGGGSGGGSSQYYDYDI |
| | | PLFMYGQISPNCAPECNCPHSYPTAMYCDDLKLKSVPMVPPGI |
| | | KYLYLRNNQIDHIDEKAFENVTDLQWLILDHNLLENSKIKGKV |
| | | FSKLKQLKKLHINYNNLTESVGPLPKSLQDLQLTNNKISKLGSF |
| | | DGLVNLTFIYLQHNQLKEDAVSASLKGLKSLEYLDLSFNQMSK |
| | | LPAGLPTSLLTLYLDNNKISNIPDEYFKRFTGLQYLRLSHNELA |
| | | DSGVPGNSFNISSLLELDLSYNKLKSIPTVNENLENYYLEVNEL |
| | | EKFDVKSFCKILGPLSYSKIKHLRLDGNPLTQSSLPPDMYECLR |
| | | VANEITVNGGGSGGGSEAHKSEIAHRYNDLGEQHFKGLVLIAF |
| | | SQYLQKCSYDEHAKLVQEVTDFAKTCVADESAANCDKSLHTL |
| | | FGDKLCAIPNLRENYGELADCCTKQEPERNECFLQHKDDNPSL |
| | | PPFERPEAEAMCTSFKENPTTFMGHYLHEVARRHPYFYAPELL |
| | | YYAEQYNEILTQCCAEADKESCLTPKLDGVKEKALVSSVRQR |
| | | MKCSSMQKFGERAFKAWAVARLSQTFPNADFAEITKLATDLT |
| | | KVNKECCHGDLLECADDRAELAKYMCENQATISSKLQTCCDK |
| | | PLLKKAHCLSEVEHDTMPADLPAIAADFVEDQEVCKNYAEAK |
| | | DVFLGTFLYEYSRRHPDYSVSLLLRLAKKYEATLEKCCAEANP |
| | | PACYGTVLAEFQPLVEEPKNLVKTNCDLYEKLGEYGFQNAILV |
| | | RYTQKAPQVSTPTLVEAARNLGRVGTKCCTLPEDQRLPCVEDY |
| | | LSAILNRVCLLQEKTPVSEHVTKCCSGSLVERRPCFSALTVDET |
| | | YVPKEFKAETFTFHSDICTLPEKEKQIKKQTALAELVKHKPKAT |
| | | AEQLKTVMDDFAQFLDTCCKAADKDTCFSTEGPNLVTRCKDA |
| | | LAGGGSAPTSSSTSSSTAEAQQQQQQQQQQQQHLEQLLMDLQ |
| | | ELLSRMENYRNLKLPRMLTFKFYLPKQATELKDLQCLEDELGP |
| | | LRHVLDLTQSKSFQLEDAENFISNIRVTVVKLKGSDNTFECQFD |
| | | DESATVVDFLRRWIAFCQSIISTSPQHHHHHHHHHH |
| 32 | 12-LAIR- | MRVPAQLLGLLLLWLPGARCAMWELEKDVYVVEVDWTPDAP |
| | MSA H464Q- | GETVNLTCDTPEEDDITWTSDQRHGVIGSGKTLTITVKEFLDAG |
| | 2 | QYTCHKGGETLSHSHLLLHKKENGIWSTEILKNFKNKTFLKCE |
| | | APNYSGRFTCSWLVQRNMDLKFNIKSSSSSPDSRAVTCGMASL |
| | | SAEKVTLDQRDYEKYSVSCQEDVTCPTAEETLPIELALEARQQ |
| | | NKYENYSTSFFIRDIIKPDPPKNLQMKPLKNSQVEVSWEYPDSW |
| | | STPHSYFSLKFFVRIQRKKEKMKETEEGCNQKGAFLVEKTSTE |
| | | VQCKGGNVCVQAQDRYYNSSCSKWACVPCRVRSGGSGGGSG |
| | | GGSGGGSRVIPVSGPARCLSQSRNLLKTTDDMVKTAREKLKHY |
| | | SCTAEDIDHEDITRDQTSTLKTCLPLELHKNESCLATRETSSTTR |
| | | GSCLPPQKTSLMMTLCLGSIYEDLKMYQTEFQAINAALQNHNH |
| | | QQIILDKGMLVAIDELMQSLNHNGETLRQKPPVGEADPYRVK |
| | | MKLCILLHAFSTRVVTINRVMGYLSSAGGGSGGGSQEGSLPDIT |
| | | IFPNSSLMISQGTFVTVVCSYSDKHDLYNMVRLEKDGSTFMEK |
| | | STEPYKTEDEFEIGPVNETITGHYSCIYSKGITWSERSKTLELKVI |
| | | KENVIQTPAPGPTSDTSWLKTYSIYGGGSGGGSEAHKSEIAHRY |
| | | NDLGEQHFKGLVLIAFSQYLQKCSYDEHAKLVQEVTDFAKTC |
| | | VADESAANCDKSLHTLFGDKLCAIPNLRENYGELADCCTKQEP |
| | | ERNECFLQHKDDNPSLPPFERPEAEAMCTSFKENPTTFMGHYL |
| | | HEVARRHPYFY APELLYYAEQYNEILTQCCAEADKESCLTPKL |
| | | DGVKEKALVSSVRQRMKCSSMQKFGERAFKAWAVARLSQTF |
| | | PNADFAEITKLATDLTKVNKECCHGDLLECADDRAELAKYMC |
| | | ENQATISSKLQTCCDKPLLKKAHCLSEVEHDTMPADLPAIAAD |
| | | FVEDQEVCKNYAEAKDVFLGTFLYEYSRRHPDYSVSLLLRLAK |
| | | KYEATLEKCCAEANPPACYGTVLAEFQPLVEEPKNLVKTNCDL |
| | | YEKLGEYGFQNAILVRYTQKAPQVSTPTLVEAARNLGRVGTK |
| | | CCTLPEDQRLPCVEDYLSAILNRVCLLQEKTPVSEHVTKCCSGS |
| | | LVERRPCFSALTVDETYVPKEFKAETFTFHSDICTLPEKEKQIKK |
| | | QTALAELVKHKPKATAEQLKTVMDDFAQFLDTCCKAADKDT |
| | | CFSTEGPNLVTRCKDALAGGGSAPTSSSTSSSTAEAQQQQQQQ |
| | | QQQQQHLEQLLMDLQELLSRMENYRNLKLPRMLTFKFYLPKQ |
| | | ATELKDLQCLEDELGPLRHVLDLTQSKSFQLEDAENFISNIRVT |
| | | VVKLKGSDNTFECQFDDESATVVDFLRRWIAFCQSIISTSPQHH |
| | | HHHHHHHH |
| 33 | 2-LAIR- | MRVPAQLLGLLLLWLPGARCAAPTSSSTSSSTAEAQQQQQQQ |
| | MSA-12 | QQQQQHLEQLLMDLQELLSRMENYRNLKLPRMLTFKFYLPKQ |
| | | ATELKDLQCLEDELGPLRHVLDLTQSKSFQLEDAENFISNIRVT |
| | | VVKLKGSDNTFECQFDDESATVVDFLRRWIAFCQSIISTSPQGG |
| | | GSGGGSQEGSLPDITIFPNSSLMISQGTFVTVVCSYSDKHDLYN |
| | | MVRLEKDGSTFMEKSTEPYKTEDEFEIGPVNETITGHYSCIYSK |
| | | GITWSERSKTLELKVIKENVIQTPAPGPTSDTSWLKTYSIYGGGS |
| | | GGGSEAHKSEIAHRYNDLGEQHFKGLVLIAFSQYLQKCSYDEH |
| | | AKLVQEVTDFAKTCVADESAANCDKSLHTLFGDKLCAIPNLRE |
| | | NYGELADCCTKQEPERNECFLQHKDDNPSLPPFERPEAEAMCT |
| | | SFKENPTTFMGHYLHEVARRHPYFYAPELLYYAEQYNEILTQC |
| | | CAEADKESCLTPKLDGVKEKALVSSVRQRMKCSSMQKFGERA |
| | | FKAWAVARLSQTFPNADFAEITKLATDLTKVNKECCHGDLLEC |
| | | ADDRAELAKYMCENQATISSKLQTCCDKPLLKKAHCLSEVEH |
| | | DTMPADLPAIAADFVEDQEVCKNYAEAKDVFLGTFLYEYSRR |
| | | HPDYSVSLLLRLAKKYEATLEKCCAEANPPACYGTVLAEFQPL |
| | | VEEPKNLVKTNCDLYEKLGEYGFQNAILVRYTQKAPQVSTPTL |
| | | VEAARNLGRVGTKCCTLPEDQRLPCVEDYLSAILNRVCLLHEK |
| | | TPVSEHVTKCCSGSLVERRPCFSALTVDETYVPKEFKAETFTFH |
| | | SDICTLPEKEKQIKKQTALAELVKHKPKATAEQLKTVMDDFAQ |
| | | FLDTCCKAADKDTCFSTEGPNLVTRCKDALAGGGSMWELEKD |
| | | VYVVEVDWTPDAPGETVNLTCDTPEEDDITWTSDQRHGVIGS |
| | | GKTLTITVKEFLDAGQYTCHKGGETLSHSHLLLHKKENGIWST |
| | | EILKNFKNKTFLKCEAPNYSGRFTCSWLVQRNMDLKFNIKSSSS |
| | | SPDSRAVTCGMASLSAEKVTLDQRDYEKYSVSCQEDVTCPTAE |
| | | ETLPIELALEARQQNKYENYSTSFFIRDIIKPDPPKNLQMKPLKN |
| | | SQVEVSWEYPDSWSTPHSYFSLKFFVRIQRKKEKMKETEEGCN |
| | | QKGAFLVEKTSTEVQCKGGNVCVQAQDRYYNSSCSKWACVP |
| | | CRVRSGGSGGGSGGGSGGGSRVIPVSGPARCLSQSRNLLKTTD |
| | | DMVKTAREKLKHYSCTAEDIDHEDITRDQTSTLKTCLPLELHK |
| | | NESCLATRETSSTTRGSCLPPQKTSLMMTLCLGSIYEDLKMYQT |
| | | EFQAINAALQNHNHQQIILDKGMLVAIDELMQSLNHNGETLRQ |
| | | KPPVGEADPYRVKMKLCILLHAFSTRVVTINRVMGYLSSAHHH |
| | | HHHHHHH |
| 34 | 2-MSA- | MRVPAQLLGLLLLWLPGARCAAPTSSSTSSSTAEAQQQQQQQ |
| | LAIR-12 | QQQQQHLEQLLMDLQELLSRMENYRNLKLPRMLTFKFYLPKQ |
| | | ATELKDLQCLEDELGPLRHVLDLTQSKSFQLEDAENFISNIRVT |
| | | VVKLKGSDNTFECQFDDESATVVDFLRRWIAFCQSIISTSPQGG |
| | | GSEAHKSEIAHRYNDLGEQHFKGLVLIAFSQYLQKCSYDEHAK |
| | | LVQEVTDFAKTCVADESAANCDKSLHTLFGDKLCAIPNLRENY |
| | | GELADCCTKQEPERNECFLQHKDDNPSLPPFERPEAEAMCTSF |
| | | KENPTTFMGHYLHEVARRHPYFYAPELLYYAEQYNEILTQCCA |
| | | EADKESCLTPKLDGVKEKALVSSVRQRMKCSSMQKFGERAFK |
| | | AWAVARLSQTFPNADFAEITKLATDLTKVNKECCHGDLLECA |
| | | DDRAELAKYMCENQATISSKLQTCCDKPLLKKAHCLSEVEHD |
| | | TMPADLPAIAADFVEDQEVCKNYAEAKDVFLGTFLYEYSRRHP |
| | | DYSVSLLLRLAKKYEATLEKCCAEANPPACYGTVLAEFQPLVE |
| | | EPKNLVKTNCDLYEKLGEYGFQNAILVRYTQKAPQVSTPTLVE |
| | | AARNLGRVGTKCCTLPEDQRLPCVEDYLSAILNRVCLLHEKTP |
| | | VSEHVTKCCSGSLVERRPCFSALTVDETYVPKEFKAETFTFHSD |
| | | ICTLPEKEKQIKKQTALAELVKHKPKATAEQLKTVMDDFAQFL |
| | | DTCCKAADKDTCFSTEGPNLVTRCKDALAGGGSGGGSQEGSL |
| | | PDITIFPNSSLMISQGTFVTVVCSYSDKHDLYNMVRLEKDGSTF |
| | | MEKSTEPYKTEDEFEIGPVNETITGHYSCIYSKGITWSERSKTLE |
| | | LKVIKENVIQTPAPGPTSDTSWLKTYSIYGGGSGGGSMWELEK |
| | | DVYVVEVDWTPDAPGETVNLTCDTPEEDDITWTSDQRHGVIG |
| | | SGKTLTITVKEFLDAGQYTCHKGGETLSHSHLLLHKKENGIWS |
| | | TEILKNFKNKTFLKCEAPNYSGRFTCSWLVQRNMDLKFNIKSSS |
| | | SSPDSRAVTCGMASLSAEKVTLDQRDYEKYSVSCQEDVTCPTA |
| | | EETLPIELALEARQQNKYENYSTSFFIRDIIKPDPPKNLQMKPLK |
| | | NSQVEVSWEYPDSWSTPHSYFSLKFFVRIQRKKEKMKETEEGC |
| | | NQKGAFLVEKTSTEVQCKGGNVCVQAQDRYYNSSCSKWACV |
| | | PCRVRSGGSGGGSGGGSGGGSRVIPVSGPARCLSQSRNLLKTT |
| | | DDMVKTAREKLKHYSCTAEDIDHEDITRDQTSTLKTCLPLELH |
| | | KNESCLATRETSSTTRGSCLPPQKTSLMMTLCLGSIYEDLKMY |
| | | QTEFQAINAALQNHNHQQIILDKGMLVAIDELMQSLNHNGETL |
| | | RQKPPVGEADPYRVKMKLCILLHAFSTRVVTINRVMGYLSSAH |
| | | HHHHHHHHH |
| 35 | 2-LAIR- | MRVPAQLLGLLLLWLPGARCAAPTSSSTSSSTAEAQQQQQQQ |
| | ABD-12 | QQQQQHLEQLLMDLQELLSRMENYRNLKLPRMLTFKFYLPKQ |
| | 2-ABD- | ATELKDLQCLEDELGPLRHVLDLTQSKSFQLEDAENFISNIRVT |
| | | VVKLKGSDNTFECQFDDESATVVDFLRRWIAFCQSIISTSPQGG |
| | | GSGGGSQEGSLPDITIFPNSSLMISQGTFVTVVCSYSDKHDLYN |
| | | MVRLEKDGSTFMEKSTEPYKTEDEFEIGPVNETITGHYSCIYSK |
| | | GITWSERSKTLELKVIKENVIQTPAPGPTSDTSWLKTYSIYGGGS |
| | | GGGSKVQLVESGGGLVQPGGSLRLSCAASGFTFSSFGMTWVR |
| | | QAPGKGLEWVSSISGSGSDTLYADSVRGRFTISRDNSKNTLYLQ |
| | | MNSLRAEDTAVYYCTIGGSLSPSSQGTLVTVSSGGGSMWELEK |
| | | DVYVVEVDWTPDAPGETVNLTCDTPEEDDITWTSDQRHGVIG |
| | | SGKTLTITVKEFLDAGQYTCHKGGETLSHSHLLLHKKENGIWS |
| | | TEILKNFKNKTFLKCEAPNYSGRFTCSWLVQRNMDLKFNIKSSS |
| | | SSPDSRAVTCGMASLSAEKVTLDQRDYEKYSVSCQEDVTCPTA |
| | | EETLPIELALEARQQNKYENYSTSFFIRDIIKPDPPKNLQMKPLK |
| | | NSQVEVSWEYPDSWSTPHSYFSLKFFVRIQRKKEKMKETEEGC |
| | | PCRVRSGGSGGGSGGGSGGGSRVIPVSGPARCLSQSRNLLKTT |
| | | DDMVKTAREKLKHYSCTAEDIDHEDITRDQTSTLKTCLPLELH |
| | | KNESCLATRETSSTTRGSCLPPQKTSLMMTLCLGSIYEDLKMY |
| | | QTEFQAINAALQNHNHQQIILDKGMLVAIDELMQSLNHNGETL |
| | | RQKPPVGEADPYRVKMKLCILLHAFSTRVVTINRVMGYLSSAH |
| | | HHHHHHHHH |
| 36 | LAIR-12 | MRVPAQLLGLLLLWLPGARCAAPTSSSTSSSTAEAQQQQQQQ |
| | | QQQQQHLEQLLMDLQELLSRMENYRNLKLPRMLTFKFYLPKQ |
| | | ATELKDLQCLEDELGPLRHVLDLTQSKSFQLEDAENFISNIRVT |
| | | VVKLKGSDNTFECQFDDESATVVDFLRRWIAFCQSIISTSPQGG |
| | | GSKVQLVESGGGLVQPGGSLRLSCAASGFTFSSFGMTWVRQAP |
| | | GKGLEWVSSISGSGSDTLYADSVRGRFTISRDNSKNTLYLQMN |
| | | SLRAEDTAVYYCTIGGSLSPSSQGTLVTVSSGGGSGGGSQEGSL |
| | | PDITIFPNSSLMISQGTFVTVVCSYSDKHDLYNMVRLEKDGSTF |
| | | MEKSTEPYKTEDEFEIGPVNETITGHYSCIYSKGITWSERSKTLE |
| | | LKVIKENVIQTPAPGPTSDTSWLKTYSIYGGGSGGGSMWELEK |
| | | DVYVVEVDWTPDAPGETVNLTCDTPEEDDITWTSDQRHGVIG |
| | | SGKTLTITVKEFLDAGQYTCHKGGETLSHSHLLLHKKENGIWS |
| | | TEILKNFKNKTFLKCEAPNYSGRFTCSWLVQRNMDLKFNIKSSS |
| | | SSPDSRAVTCGMASLSAEKVTLDQRDYEKYSVSCQEDVTCPTA |
| | | EETLPIELALEARQQNKYENYSTSFFIRDIIKPDPPKNLQMKPLK |
| | | NSQVEVSWEYPDSWSTPHSYFSLKFFVRIQRKKEKMKETEEGC |
| | | NQKGAFLVEKTSTEVQCKGGNVCVQAQDRYYNSSCSKWACV |
| | | PCRVRSGGSGGGSGGGSGGGSRVIPVSGPARCLSQSRNLLKTT |
| | | DDMVKTAREKLKHYSCTAEDIDHEDITRDQTSTLKTCLPLELH |
| | | KNESCLATRETSSTTRGSCLPPQKTSLMMTLCLGSIYEDLKMY |
| | | QTEFQAINAALQNHNHQQIILDKGMLVAIDELMQSLNHNGETL |
| | | RQKPPVGEADPYRVKMKLCILLHAFSTRVVTINRVMGYLSSAH |
| | | HHHHHHHHH |
| 37 | 12-LAIR- | MRVPAQLLGLLLLWLPGARCAMWELEKDVYVVEVDWTPDAP |
| | MSA-2 | GETVNLTCDTPEEDDITWTSDQRHGVIGSGKTLTITVKEFLDAG |
| | (normal | QYTCHKGGETLSHSHLLLHKKENGIWSTEILKNFKNKTFLKCE |
| | linkers) | APNYSGRFTCSWLVQRNMDLKFNIKSSSSSPDSRAVTCGMASL |
| | | SAEKVTLDQRDYEKYSVSCQEDVTCPTAEETLPIELALEARQQ |
| | | NKYENYSTSFFIRDIIKPDPPKNLQMKPLKNSQVEVSWEYPDSW |
| | | STPHSYFSLKFFVRIQRKKEKMKETEEGCNQKGAFLVEKTSTE |
| | | VQCKGGNVCVQAQDRYYNSSCSKWACVPCRVRSGGSGGGSG |
| | | GGSGGGSRVIPVSGPARCLSQSRNLLKTTDDMVKTAREKLKHY |
| | | SCTAEDIDHEDITRDQTSTLKTCLPLELHKNESCLATRETSSTTR |
| | | GSCLPPQKTSLMMTLCLGSIYEDLKMYQTEFQAINAALQNHNH |
| | | QQIILDKGMLVAIDELMQSLNHNGETLRQKPPVGEADPYRVK |
| | | MKLCILLHAFSTRVVTINRVMGYLSSAGGGGSGGGSQEGSLPD |
| | | ITIFPNSSLMISQGTFVTVVCSYSDKHDLYNMVRLEKDGSTFME |
| | | KSTEPYKTEDEFEIGPVNETITGHYSCIYSKGITWSERSKTLELK |
| | | VIKENVIQTPAPGPTSDTSWLKTYSIYGGGGSGGGSEAHKSEIA |
| | | HRYNDLGEQHFKGLVLIAFSQYLQKCSYDEHAKLVQEVTDFA |
| | | KTCVADESAANCDKSLHTLFGDKLCAIPNLRENYGELADCCTK |
| | | QEPERNECFLQHKDDNPSLPPFERPEAEAMCTSFKENPTTFMG |
| | | HYLHEVARRHPYFYAPELLYYAEQYNEILTQCCAEADKESCLT |
| | | PKLDGVKEKALVSSVRQRMKCSSMQKFGERAFKAWAVARLS |
| | | QTFPNADFAEITKLATDLTKVNKECCHGDLLECADDRAELAKY |
| | | MCENQATISSKLQTCCDKPLLKKAHCLSEVEHDTMPADLPAIA |
| | | ADFVEDQEVCKNYAEAKDVFLGTFLYEYSRRHPDYSVSLLLRL |
| | | AKKYEATLEKCCAEANPPACYGTVLAEFQPLVEEPKNLVKTN |
| | | CDLYEKLGEYGFQNAILVRYTQKAPQVSTPTLVEAARNLGRV |
| | | GTKCCTLPEDQRLPCVEDYLSAILNRVCLLHEKTPVSEHVTKC |
| | | CSGSLVERRPCFSALTVDETYVPKEFKAETFTFHSDICTLPEKEK |
| | | QIKKQTALAELVKHKPKATAEQLKTVMDDFAQFLDTCCKAAD |
| | | KDTCFSTEGPNLVTRCKDALAGGGGSGGGSAPTSSSTSSSTAEA |
| | | QQQQQQQQQQQQHLEQLLMDLQELLSRMENYRNLKLPRMLT |
| | | FKFYLPKQATELKDLQCLEDELGPLRHVLDLTQSKSFQLEDAE |
| | | NFISNIRVTVVKLKGSDNTFECQFDDESATVVDFLRRWIAFCQS |
| | | IISTSPQHHHHHHHHHH |

TABLE 6
Exemplary human bi-functional linear constructs

| SEQ ID | Construct | |
|---|---|---|
| NO: | Name | Sequence |
| 38 | 12-LAIR1- | MRVPAQLLGLLLLWLPGARCAIWELKKDVYVVELDWYPDAP |
| | HSA-2 | GEMVVLTCDTPEEDGITWTLDQSSEVLGSGKTLTIQVKEFGDA |
| | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSQEEDLPRPSISAEP |
| | | GTVIPLGSHVTFVCRGPVGVQTFRLERESRSTYNDTEDVSQASP |
| | | SESEARFRIDSVSEGNAGPYRCIYYKPPKWSEQSDYLELLVKET |
| | | SGGPDSPDTEPGSSAGPTQRPSDNSHNEHAPASQGLKAEHLYG |
| | | GGSGGGSDAHKSEVAHRFKDLGEENFKALVLIAFAQYLQQCPF |
| | | EDHVKLVNEVTEFAKTCVADESAENCDKSLHTLFGDKLCTVA |
| | | TLRETYGEMADCCAKQEPERNECFLQHKDDNPNLPRLVRPEV |
| | | DVMCTAFHDNEETFLKKYLYEIARRHPYFYAPELLFFAKRYKA |
| | | AFTECCQAADKAACLLPKLDELRDEGKASSAKQRLKCASLQK |
| | | FGERAFKAWAVARLSQRFPKAEFAEVSKLVTDLTKVHTECCH |
| | | GDLLECADDRADLAKYICENQDSISSKLKECCEKPLLEKSHCIA |
| | | EVENDEMPADLPSLAADFVESKDVCKNYAEAKDVFLGMFLYE |
| | | YARRHPDYSVVLLLRLAKTYETTLEKCCAAADPHECYAKVFD |
| | | EFKPLVEEPQNLIKQNCELFEQLGEYKFQNALLVRYTKKVPQV |
| | | STPTLVEVSRNLGKVGSKCCKHPEAKRMPCAEDYLSVVLNQL |
| | | CVLHEKTPVSDRVTKCCTESLVNRRPCFSALEVDETYVPKEFN |
| | | AETFTFHADICTLSEKERQIKKQTALVELVKHKPKATKEQLKA |
| | | VMDDFAAFVEKCCKADDKETCFAEEGKKLVAASQAALGLGG |
| | | GSAPTSSSTKKTQLQLEHLLLDLQMILNGINNYKNPKLTRMLTF |
| | | KFYMPKKATELKHLQCLEEELKPLEEVLNLAQSKNFHLRPRDL |
| | | ISNINVIVLELKGSETTFMCEYADETATIVEFLNRWITFCQSIIST |
| | | LTHHHHHHHHHH |
| 39 | 12-LAIR2- | MRVPAQLLGLLLLWLPGARCAIWELKKDVYVVELDWYPDAP |
| | HSA-2 | GEMVVLTCDTPEEDGITWTLDQSSEVLGSGKTLTIQVKEFGDA |
| | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSQEGALPRPSISAEP |
| | | GTVISPGSHVTFMCRGPVGVQTFRLEREDRAKYKDSYNVFRLG |
| | | PSESEARFHIDSVSEGNAGLYRCLYYKPPGWSEHSDFLELLVKE |
| | | SSGGPDSPDTEPGSSAGTVPGTEASGFDAPGGGSGGGSDAHKS |
| | | EVAHRFKDLGEENFKALVLIAFAQYLQQCPFEDHVKLVNEVTE |
| | | FAKTCVADESAENCDKSLHTLFGDKLCTVATLRETYGEMADC |
| | | CAKQEPERNECFLQHKDDNPNLPRLVRPEVDVMCTAFHDNEE |
| | | TFLKKYLYEIARRHPYFYAPELLFFAKRYKAAFTECCQAADKA |
| | | ACLLPKLDELRDEGKASSAKQRLKCASLQKFGERAFKAWAVA |
| | | RLSQRFPKAEFAEVSKLVTDLTKVHTECCHGDLLECADDRADL |
| | | AKYICENQDSISSKLKECCEKPLLEKSHCIAEVENDEMPADLPS |
| | | LAADFVESKDVCKNYAEAKDVFLGMFLYEYARRHPDYSVVLL |
| | | LRLAKTYETTLEKCCAAADPHECYAKVFDEFKPLVEEPQNLIK |
| | | QNCELFEQLGEYKFQNALLVRYTKKVPQVSTPTLVEVSRNLGK |
| | | VGSKCCKHPEAKRMPCAEDYLSVVLNQLCVLHEKTPVSDRVT |
| | | KCCTESLVNRRPCFSALEVDETYVPKEFNAETFTFHADICTLSE |
| | | KERQIKKQTALVELVKHKPKATKEQLKAVMDDFAAFVEKCCK |
| | | ADDKETCFAEEGKKLVAASQAALGLGGGSAPTSSSTKKTQLQL |
| | | EHLLLDLQMILNGINNYKNPKLTRMLTFKFYMPKKATELKHLQ |
| | | CLEEELKPLEEVLNLAQSKNFHLRPRDLISNINVIVLELKGSETT |
| | | FMCEYADETATIVEFLNRWITFCQSIISTLTHHHHHHHHHH |
| 40 | 12-Lum- | MRVPAQLLGLLLLWLPGARCAIWELKKDVYVVELDWYPDAP |
| | HSA-2 | GEMVVLTCDTPEEDGITWTLDQSSEVLGSGKTLTIQVKEFGDA |
| | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSSGQYYDYDFPLSI |
| | | YGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGSGGGSDAHKSEVAHRFKDLGEENFKALVLIAFAQYLQQ |
| | | CPFEDHVKLVNEVTEFAKTCVADESAENCDKSLHTLFGDKLCT |
| | | VATLRETYGEMADCCAKQEPERNECFLQHKDDNPNLPRLVRP |
| | | EVDVMCTAFHDNEETFLKKYLYEIARRHPYFYAPELLFFAKRY |
| | | KAAFTECCQAADKAACLLPKLDELRDEGKASSAKQRLKCASL |
| | | QKFGERAFKAWAVARLSQRFPKAEFAEVSKLVTDLTKVHTEC |
| | | CHGDLLECADDRADLAKYICENQDSISSKLKECCEKPLLEKSH |
| | | CIAEVENDEMPADLPSLAADFVESKDVCKNYAEAKDVFLGMF |
| | | LYEYARRHPDYSVVLLLRLAKTYETTLEKCCAAADPHECYAK |
| | | VFDEFKPLVEEPQNLIKQNCELFEQLGEYKFQNALLVRYTKKV |
| | | PQVSTPTLVEVSRNLGKVGSKCCKHPEAKRMPCAEDYLSVVL |
| | | NQLCVLHEKTPVSDRVTKCCTESLVNRRPCFSALEVDETYVPK |
| | | EFNAETFTFHADICTLSEKERQIKKQTALVELVKHKPKATKEQL |
| | | KAVMDDFAAFVEKCCKADDKETCFAEEGKKLVAASQAALGL |
| | | GGGSAPTSSSTKKTQLQLEHLLLDLQMILNGINNYKNPKLTRM |
| | | LTFKFYMPKKATELKHLQCLEEELKPLEEVLNLAQSKNFHLRP |
| | | RDLISNINVIVLELKGSETTFMCEYADETATIVEFLNRWITFCQSI |
| | | ISTLTHHHHHHHHHH |
| 41 | 12-LAIR1- | MRVPAQLLGLLLLWLPGARCAIWELKKDVYVVELDWYPDAP |
| | ABD-2 | GEMVVLTCDTPEEDGITWTLDQSSEVLGSGKTLTIQVKEFGDA |
| | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSQEEDLPRPSISAEP |
| | | GTVIPLGSHVTFVCRGPVGVQTFRLERESRSTYNDTEDVSQASP |
| | | SESEARFRIDSVSEGNAGPYRCIYYKPPKWSEQSDYLELLVKET |
| | | SGGPDSPDTEPGSSAGPTQRPSDNSHNEHAPASQGLKAEHLYG |
| | | GGSGGGSKVQLVESGGGLVQPGGSLRLSCAASGFTFSSFGMT |
| | | WVRQAPGKGLEWVSSISGSGSDTLYADSVRGRFTISRDNSKNT |
| | | LYLQMNSLRAEDTAVYYCTIGGSLSPSSQGTLVTVSSGGGSAP |
| | | TSSSTKKTQLQLEHLLLDLQMILNGINNYKNPKLTRMLTFKFY |
| | | MPKKATELKHLQCLEEELKPLEEVLNLAQSKNFHLRPRDLISNI |
| | | NVIVLELKGSETTFMCEYADETATIVEFLNRWITFCQSIISTLTH |
| | | HHHHHHHHH |
| 42 | 12-LAIR2- | MRVPAQLLGLLLLWLPGARCAIWELKKDVYVVELDWYPDAP |
| | ABD-2 | GEMVVLTCDTPEEDGITWTLDQSSEVLGSGKTLTIQVKEFGDA |
| | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSQEGALPRPSISAEP |
| | | GTVISPGSHVTFMCRGPVGVQTFRLEREDRAKYKDSYNVFRLG |
| | | PSESEARFHIDSVSEGNAGLYRCLYYKPPGWSEHSDFLELLVKE |
| | | SSGGPDSPDTEPGSSAGTVPGTEASGFDAPGGGSGGGSKVQLV |
| | | ESGGGLVQPGGSLRLSCAASGFTFSSFGMTWVRQAPGKGLEW |
| | | VSSISGSGSDTLYADSVRGRFTISRDNSKNTLYLQMNSLRAEDT |
| | | AVYYCTIGGSLSPSSQGTLVTVSSGGGSAPTSSSTKKTQLQLEH |
| | | LLLDLQMILNGINNYKNPKLTRMLTFKFYMPKKATELKHLQCL |
| | | EEELKPLEEVLNLAQSKNFHLRPRDLISNINVIVLELKGSETTFM |
| | | CEYADETATIVEFLNRWITFCQSIISTLTHHHHHHHHHH |
| 43 | 12-Lum- | MRVPAQLLGLLLLWLPGARCAIWELKKDVYVVELDWYPDAP |
| | ABD-2 | GEMVVLTCDTPEEDGITWTLDQSSEVLGSGKTLTIQVKEFGDA |
| | | GQYTCHKGGEVLSHSLLLLHKKEDGIWSTDILKDQKEPKNKTF |
| | | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSSGQYYDYDFPLSI |
| | | YGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHINKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGSGGGSKVQLVESGGGLVQPGGSLRLSCAASGFTFSSFGM |
| | | TWVRQAPGKGLEWVSSISGSGSDTLYADSVRGRFTISRDNSKN |
| | | TLYLQMNSLRAEDTAVYYCTIGGSLSPSSQGTLVTVSSGGGSA |
| | | PTSSSTKKTQLQLEHLLLDLQMILNGINNYKNPKLTRMLTFKFY |
| | | MPKKATELKHLQCLEEELKPLEEVLNLAQSKNFHLRPRDLISNI |
| | | NVIVLELKGSETTFMCEYADETATIVEFLNRWITFCQSIISTLTH |
| | | HHHHHHHHH |
| 44 | 12-LAIR1- | MRVPAQLLGLLLLWLPGARCAIWELKKDVYVVELDWYPDAP |
| | HSA-2 | GEMVVLTCDTPEEDGITWTLDQSSEVLGSGKTLTIQVKEFGDA |
| | (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSQEEDLPRPSISAE |
| | | PGTVIPLGSHVTFVCRGPVGVQTFRLERESRSTYNDTEDVSQAS |
| | | PSESEARFRIDSVSEGNAGPYRCIYYKPPKWSEQSDYLELLVKE |
| | | TSGGPDSPDTEPGSSAGPTQRPSDNSHNEHAPASQGLKAEHLY |
| | | GGGGSGGGSDAHKSEVAHRFKDLGEENFKALVLIAFAQYLQQ |
| | | CPFEDHVKLVNEVTEFAKTCVADESAENCDKSLHTLFGDKLCT |
| | | VATLRETYGEMADCCAKQEPERNECFLQHKDDNPNLPRLVRP |
| | | EVDVMCTAFHDNEETFLKKYLYEIARRHPYFYAPELLFFAKRY |
| | | KAAFTECCQAADKAACLLPKLDELRDEGKASSAKQRLKCASL |
| | | QKFGERAFKAWAVARLSQRFPKAEFAEVSKLVTDLTKVHTEC |
| | | CHGDLLECADDRADLAKYICENQDSISSKLKECCEKPLLEKSH |
| | | CIAEVENDEMPADLPSLAADFVESKDVCKNYAEAKDVFLGMF |
| | | LYEYARRHPDYSVVLLLRLAKTYETTLEKCCAAADPHECYAK |
| | | VFDEFKPLVEEPQNLIKQNCELFEQLGEYKFQNALLVRYTKKV |
| | | PQVSTPTLVEVSRNLGKVGSKCCKHPEAKRMPCAEDYLSVVL |
| | | NQLCVLHEKTPVSDRVTKCCTESLVNRRPCFSALEVDETYVPK |
| | | EFNAETFTFHADICTLSEKERQIKKQTALVELVKHKPKATKEQL |
| | | KAVMDDFAAFVEKCCKADDKETCFAEEGKKLVAASQAALGL |
| | | GGGGSGGGSAPTSSSTKKTQLQLEHLLLDLQMILNGINNYKNP |
| | | KLTRMLTFKFYMPKKATELKHLQCLEEELKPLEEVLNLAQSKN |
| | | FHLRPRDLISNINVIVLELKGSETTFMCEYADETATIVEFLNRWI |
| | | TFCQSIISTLTHHHHHHHHHH |
| 45 | 12-LAIR2- | MRVPAQLLGLLLLWLPGARCAIWELKKDVYVVELDWYPDAP |
| | HSA-2 | GEMVVLTCDTPEEDGITWTLDQSSEVLGSGKTLTIQVKEFGDA |
| | (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSQEGALPRPSISAE |
| | | PGTVISPGSHVTFMCRGPVGVQTFRLEREDRAKYKDSYNVFRL |
| | | GPSESEARFHIDSVSEGNAGLYRCLYYKPPGWSEHSDFLELLVK |
| | | ESSGGPDSPDTEPGSSAGTVPGTEASGFDAPGGGGSGGGSDAH |
| | | KSEVAHRFKDLGEENFKALVLIAFAQYLQQCPFEDHVKLVNEV |
| | | TEFAKTCVADESAENCDKSLHTLFGDKLCTVATLRETYGEMA |
| | | DCCAKQEPERNECFLQHKDDNPNLPRLVRPEVDVMCTAFHDN |
| | | EETFLKKYLYEIARRHPYFYAPELLFFAKRYKAAFTECCQAAD |
| | | KAACLLPKLDELRDEGKASSAKQRLKCASLQKFGERAFKAWA |
| | | VARLSQRFPKAEFAEVSKLVTDLTKVHTECCHGDLLECADDR |
| | | ADLAKYICENQDSISSKLKECCEKPLLEKSHCIAEVENDEMPAD |
| | | LPSLAADFVESKDVCKNYAEAKDVFLGMFLYEYARRHPDYSV |
| | | VLLLRLAKTYETTLEKCCAAADPHECYAKVFDEFKPLVEEPQN |
| | | LIKQNCELFEQLGEYKFQNALLVRYTKKVPQVSTPTLVEVSRN |
| | | LGKVGSKCCKHPEAKRMPCAEDYLSVVLNQLCVLHEKTPVSD |
| | | RVTKCCTESLVNRRPCFSALEVDETYVPKEFNAETFTFHADICT |
| | | LSEKERQIKKQTALVELVKHKPKATKEQLKAVMDDFAAFVEK |
| | | CCKADDKETCFAEEGKKLVAASQAALGLGGGGSGGGSAPTSS |
| | | STKKTQLQLEHLLLDLQMILNGINNYKNPKLTRMLTFKFYMPK |
| | | KATELKHLQCLEEELKPLEEVLNLAQSKNFHLRPRDLISNINVI |
| | | VLELKGSETTFMCEYADETATIVEFLNRWITFCQSIISTLTHHHH |
| | | HHHHHH |
| 46 | 12-Lum- | MRVPAQLLGLLLLWLPGARCAIWELKKDVYVVELDWYPDAP |
| | HSA-2 | GEMVVLTCDTPEEDGITWTLDQSSEVLGSGKTLTIQVKEFGDA |
| | (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSSGQYYDYDFPLS |
| | | IYGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSDAHKSEVAHRFKDLGEENFKALVLIAFAQYLQ |
| | | QCPFEDHVKLVNEVTEFAKTCVADESAENCDKSLHTLFGDKLC |
| | | TVATLRETYGEMADCCAKQEPERNECFLQHKDDNPNLPRLVR |
| | | PEVDVMCTAFHDNEETFLKKYLYEIARRHPYFYAPELLFFAKR |
| | | YKAAFTECCQAADKAACLLPKLDELRDEGKASSAKQRLKCAS |
| | | LQKFGERAFKAWAVARLSQRFPKAEFAEVSKLVTDLTKVHTE |
| | | CCHGDLLECADDRADLAKYICENQDSISSKLKECCEKPLLEKS |
| | | HCIAEVENDEMPADLPSLAADFVESKDVCKNYAEAKDVFLGM |
| | | FLYEYARRHPDYSVVLLLRLAKTYETTLEKCCAAADPHECYA |
| | | KVFDEFKPLVEEPQNLIKQNCELFEQLGEYKFQNALLVRYTKK |
| | | VPQVSTPTLVEVSRNLGKVGSKCCKHPEAKRMPCAEDYLSVV |
| | | LNQLCVLHEKTPVSDRVTKCCTESLVNRRPCFSALEVDETYVP |
| | | KEFNAETFTFHADICTLSEKERQIKKQTALVELVKHKPKATKEQ |
| | | LKAVMDDFAAFVEKCCKADDKETCFAEEGKKLVAASQAALG |
| | | LGGGGSGGGSAPTSSSTKKTQLQLEHLLLDLQMILNGINNYKN |
| | | PKLTRMLTFKFYMPKKATELKHLQCLEEELKPLEEVLNLAQSK |
| | | NFHLRPRDLISNINVIVLELKGSETTFMCEYADETATIVEFLNR |
| | | WITFCQSIISTLTHHHHHHHHHH |
| 47 | 12-LAIR1- | MRVPAQLLGLLLLWLPGARCAIWELKKDVYVVELDWYPDAP |
| | ABD-2 | GEMVVLTCDTPEEDGITWTLDQSSEVLGSGKTLTIQVKEFGDA |
| | (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSQEEDLPRPSISAE |
| | | PGTVIPLGSHVTFVCRGPVGVQTFRLERESRSTYNDTEDVSQAS |
| | | PSESEARFRIDSVSEGNAGPYRCIYYKPPKWSEQSDYLELLVKE |
| | | TSGGPDSPDTEPGSSAGPTQRPSDNSHNEHAPASQGLKAEHLY |
| | | GGGGSGGGSKVQLVESGGGLVQPGGSLRLSCAASGFTFSSFGM |
| | | TWVRQAPGKGLEWVSSISGSGSDTLYADSVRGRFTISRDNSKN |
| | | TLYLQMNSLRAEDTAVYYCTIGGSLSPSSQGTLVTVSSGGGGS |
| | | GGGSAPTSSSTKKTQLQLEHLLLDLQMILNGINNYKNPKLTRM |
| | | LTFKFYMPKKATELKHLQCLEEELKPLEEVLNLAQSKNFHLRP |
| | | RDLISNINVIVLELKGSETTFMCEYADETATIVEFLNRWITFCQSI |
| | | ISTLTHHHHHHHHHH |
| 48 | 12-LAIR2- | MRVPAQLLGLLLLWLPGARCAIWELKKDVYVVELDWYPDAP |
| | ABD-2 | GEMVVLTCDTPEEDGITWTLDQSSEVLGSGKTLTIQVKEFGDA |
| | (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSQEGALPRPSISAE |
| | | PGTVISPGSHVTFMCRGPVGVQTFRLEREDRAKYKDSYNVFRL |
| | | GPSESEARFHIDSVSEGNAGLYRCLYYKPPGWSEHSDFLELLVK |
| | | ESSGGPDSPDTEPGSSAGTVPGTEASGFDAPGGGGSGGGSKVQ |
| | | LVESGGGLVQPGGSLRLSCAASGFTFSSFGMTWVRQAPGKGLE |
| | | WVSSISGSGSDTLYADSVRGRFTISRDNSKNTLYLQMNSLRAE |
| | | DTAVYYCTIGGSLSPSSQGTLVTVSSGGGGSGGGSAPTSSSTKK |
| | | TQLQLEHLLLDLQMILNGINNYKNPKLTRMLTFKFYMPKKATE |
| | | LKHLQCLEEELKPLEEVLNLAQSKNFHLRPRDLISNINVIVLELK |
| | | GSETTFMCEYADETATIVEFLNRWITFCQSIISTLTHHHHHHHH |
| | | HH |
| 49 | 12-Lum- | MRVPAQLLGLLLLWLPGARCAIWELKKDVYVVELDWYPDAP |
| | ABD-2 | GEMVVLTCDTPEEDGITWTLDQSSEVLGSGKTLTIQVKEFGDA |
| | (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSSGQYYDYDFPLS |
| | | IYGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSKVQLVESGGGLVQPGGSLRLSCAASGFTFSSFG |
| | | MTWVRQAPGKGLEWVSSISGSGSDTLYADSVRGRFTISRDNSK |
| | | NTLYLQMNSLRAEDTAVYYCTIGGSLSPSSQGTLVTVSSGGGG |
| | | SGGGSAPTSSSTKKTQLQLEHLLLDLQMILNGINNYKNPKLTR |
| | | MLTFKFYMPKKATELKHLQCLEEELKPLEEVLNLAQSKNFHLR |
| | | PRDLISNINVIVLELKGSETTFMCEYADETATIVEFLNRWITFCQ |
| | | SIISTLTHHHHHHHHHH |
| 50 | 2-LAIR1- | MRVPAQLLGLLLLWLPGARCAAPTSSSTKKTQLQLEHLLLDLQ |
| | HSA-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSQEEDLPRPSISAE |
| | | PGTVIPLGSHVTFVCRGPVGVQTFRLERESRSTYNDTEDVSQAS |
| | | PSESEARFRIDSVSEGNAGPYRCIYYKPPKWSEQSDYLELLVKE |
| | | TSGGPDSPDTEPGSSAGPTQRPSDNSHNEHAPASQGLKAEHLY |
| | | GGGGSGGGSDAHKSEVAHRFKDLGEENFKALVLIAFAQYLQQ |
| | | CPFEDHVKLVNEVTEFAKTCVADESAENCDKSLHTLFGDKLCT |
| | | VATLRETYGEMADCCAKQEPERNECFLQHKDDNPNLPRLVRP |
| | | EVDVMCTAFHDNEETFLKKYLYEIARRHPYFYAPELLFFAKRY |
| | | KAAFTECCQAADKAACLLPKLDELRDEGKASSAKQRLKCASL |
| | | QKFGERAFKAWAVARLSQRFPKAEFAEVSKLVTDLTKVHTEC |
| | | CHGDLLECADDRADLAKYICENQDSISSKLKECCEKPLLEKSH |
| | | CIAEVENDEMPADLPSLAADFVESKDVCKNYAEAKDVFLGMF |
| | | LYEYARRHPDYSVVLLLRLAKTYETTLEKCCAAADPHECYAK |
| | | VFDEFKPLVEEPQNLIKQNCELFEQLGEYKFQNALLVRYTKKV |
| | | PQVSTPTLVEVSRNLGKVGSKCCKHPEAKRMPCAEDYLSVVL |
| | | NQLCVLHEKTPVSDRVTKCCTESLVNRRPCFSALEVDETYVPK |
| | | EFNAETFTFHADICTLSEKERQIKKQTALVELVKHKPKATKEQL |
| | | KAVMDDFAAFVEKCCKADDKETCFAEEGKKLVAASQAALGL |
| | | GGGGSGGGSIWELKKDVYVVELDWYPDAPGEMVVLTCDTPE |
| | | EDGITWTLDQSSEVLGSGKTLTIQVKEFGDAGQYTCHKGGEVL |
| | | SHSLLLLHKKEDGIWSTDILKDQKEPKNKTFLRCEAKNYSGRF |
| | | TCWWLTTISTDLTFSVKSSRGSSDPQGVTCGAATLSAERVRGD |
| | | NKEYEYSVECQEDSACPAAEESLPIEVMVDAVHKLKYENYTSS |
| | | FFIRDIIKPDPPKNLQLKPLKNSRQVEVSWEYPDTWSTPHSYFSL |
| | | TFCVQVQGKSKREKKDRVFTDKTSATVICRKNASISVRAQDRY |
| | | YSSSWSEWASVPCSGGSGGGSGGGSGGGSRNLPVATPDPGMF |
| | | PCLHHSQNLLRAVSNMLQKARQTLEFYPCTSEEIDHEDITKDKT |
| | | STVEACLPLELTKNESCLNSRETSFITNGSCLASRKTSFMMALC |
| | | LSSIYEDLKMYQVEFKTMNAKLLMDPKRQIFLDQNMLAVIDEL |
| | | MQALNFNSETVPQKSSLEEPDFYKTKIKLCILLHAFRIRAVTIDR |
| | | VMSYLNASHHHHHHHHHH |
| 51 | 2-LAIR2- | MRVPAQLLGLLLLWLPGARCAAPTSSSTKKTQLQLEHLLLDLQ |
| | HSA-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSQEGALPRPSISAE |
| | | PGTVISPGSHVTFMCRGPVGVQTFRLEREDRAKYKDSYNVFRL |
| | | GPSESEARFHIDSVSEGNAGLYRCLYYKPPGWSEHSDFLELLVK |
| | | ESSGGPDSPDTEPGSSAGTVPGTEASGFDAPGGGGSGGGSDAH |
| | | KSEVAHRFKDLGEENFKALVLIAFAQYLQQCPFEDHVKLVNEV |
| | | TEFAKTCVADESAENCDKSLHTLFGDKLCTVATLRETYGEMA |
| | | DCCAKQEPERNECFLQHKDDNPNLPRLVRPEVDVMCTAFHDN |
| | | EETFLKKYLYEIARRHPYFYAPELLFFAKRYKAAFTECCQAAD |
| | | KAACLLPKLDELRDEGKASSAKQRLKCASLQKFGERAFKAWA |
| | | VARLSQRFPKAEFAEVSKLVTDLTKVHTECCHGDLLECADDR |
| | | ADLAKYICENQDSISSKLKECCEKPLLEKSHCIAEVENDEMPAD |
| | | LPSLAADFVESKDVCKNYAEAKDVFLGMFLYEYARRHPDYSV |
| | | VLLLRLAKTYETTLEKCCAAADPHECYAKVFDEFKPLVEEPQN |
| | | LIKQNCELFEQLGEYKFQNALLVRYTKKVPQVSTPTLVEVSRN |
| | | LGKVGSKCCKHPEAKRMPCAEDYLSVVLNQLCVLHEKTPVSD |
| | | RVTKCCTESLVNRRPCFSALEVDETYVPKEFNAETFTFHADICT |
| | | LSEKERQIKKQTALVELVKHKPKATKEQLKAVMDDFAAFVEK |
| | | CCKADDKETCFAEEGKKLVAASQAALGLGGGGSGGGSIWELK |
| | | KDVYVVELDWYPDAPGEMVVLTCDTPEEDGITWTLDQSSEVL |
| | | GSGKTLTIQVKEFGDAGQYTCHKGGEVLSHSLLLLHKKEDGIW |
| | | STDILKDQKEPKNKTFLRCEAKNYSGRFTCWWLTTISTDLTFSV |
| | | KSSRGSSDPQGVTCGAATLSAERVRGDNKEYEYSVECQEDSAC |
| | | PAAEESLPIEVMVDAVHKLKYENYTSSFFIRDIIKPDPPKNLQLK |
| | | PLKNSRQVEVSWEYPDTWSTPHSYFSLTFCVQVQGKSKREKK |
| | | DRVFTDKTSATVICRKNASISVRAQDRYYSSSWSEWASVPCSG |
| | | GSGGGSGGGSGGGSRNLPVATPDPGMFPCLHHSQNLLRAVSN |
| | | MLQKARQTLEFYPCTSEEIDHEDITKDKTSTVEACLPLELTKNE |
| | | SCLNSRETSFITNGSCLASRKTSFMMALCLSSIYEDLKMYQVEF |
| | | KTMNAKLLMDPKRQIFLDQNMLAVIDELMQALNFNSETVPQK |
| | | SSLEEPDFYKTKIKLCILLHAFRIRAVTIDRVMSYLNASHHHHH |
| | | HHHHH |
| 52 | 2-Lum-HSA- | MRVPAQLLGLLLLWLPGARCAAPTSSSTKKTQLQLEHLLLDLQ |
| | 12 (normal | MILNGINNYKNPKLTRMLTFKFYMPKKATELKHLQCLEEELKP |
| | linkers) | LEEVLNLAQSKNFHLRPRDLISNINVIVLELKGSETTFMCEYAD |
| | | ETATIVEFLNRWITFCQSIISTLTGGGGSGGGSSGQYYDYDFPLS |
| | | IYGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSDAHKSEVAHRFKDLGEENFKALVLIAFAQYLQ |
| | | QCPFEDHVKLVNEVTEFAKTCVADESAENCDKSLHTLFGDKLC |
| | | TVATLRETYGEMADCCAKQEPERNECFLQHKDDNPNLPRLVR |
| | | PEVDVMCTAFHDNEETFLKKYLYEIARRHPYFYAPELLFFAKR |
| | | YKAAFTECCQAADKAACLLPKLDELRDEGKASSAKQRLKCAS |
| | | LQKFGERAFKAWAVARLSQRFPKAEFAEVSKLVTDLTKVHTE |
| | | CCHGDLLECADDRADLAKYICENQDSISSKLKECCEKPLLEKS |
| | | HCIAEVENDEMPADLPSLAADFVESKDVCKNYAEAKDVFLGM |
| | | FLYEYARRHPDYSVVLLLRLAKTYETTLEKCCAAADPHECYA |
| | | KVFDEFKPLVEEPQNLIKQNCELFEQLGEYKFQNALLVRYTKK |
| | | VPQVSTPTLVEVSRNLGKVGSKCCKHPEAKRMPCAEDYLSVV |
| | | LNQLCVLHEKTPVSDRVTKCCTESLVNRRPCFSALEVDETYVP |
| | | KEFNAETFTFHADICTLSEKERQIKKQTALVELVKHKPKATKEQ |
| | | LKAVMDDFAAFVEKCCKADDKETCFAEEGKKLVAASQAALG |
| | | LGGGGSGGGSIWELKKDVYVVELDWYPDAPGEMVVLTCDTP |
| | | EEDGITWTLDQSSEVLGSGKTLTIQVKEFGDAGQYTCHKGGEV |
| | | LSHSLLLLHKKEDGIWSTDILKDQKEPKNKTFLRCEAKNYSGR |
| | | FTCWWLTTISTDLTFSVKSSRGSSDPQGVTCGAATLSAERVRG |
| | | DNKEYEYSVECQEDSACPAAEESLPIEVMVDAVHKLKYENYTS |
| | | SFFIRDIIKPDPPKNLQLKPLKNSRQVEVSWEYPDTWSTPHSYFS |
| | | LTFCVQVQGKSKREKKDRVFTDKTSATVICRKNASISVRAQDR |
| | | YYSSSWSEWASVPCSGGSGGGSGGGSGGGSRNLPVATPDPGM |
| | | FPCLHHSQNLLRAVSNMLQKARQTLEFYPCTSEEIDHEDITKDK |
| | | TSTVEACLPLELTKNESCLNSRETSFITNGSCLASRKTSFMMAL |
| | | CLSSIYEDLKMYQVEFKTMNAKLLMDPKRQIFLDQNMLAVIDE |
| | | LMQALNFNSETVPQKSSLEEPDFYKTKIKLCILLHAFRIRAVTID |
| | | RVMSYLNASHHHHHHHHHH |
| 53 | 2-LAIR1- | MRVPAQLLGLLLLWLPGARCAAPTSSSTKKTQLQLEHLLLDLQ |
| | ABD-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSQEEDLPRPSISAE |
| | | PGTVIPLGSHVTFVCRGPVGVQTFRLERESRSTYNDTEDVSQAS |
| | | PSESEARFRIDSVSEGNAGPYRCIYYKPPKWSEQSDYLELLVKE |
| | | TSGGPDSPDTEPGSSAGPTQRPSDNSHNEHAPASQGLKAEHLY |
| | | GGGGSGGGSKVQLVESGGGLVQPGGSLRLSCAASGFTFSSFGM |
| | | TWVRQAPGKGLEWVSSISGSGSDTLYADSVRGRFTISRDNSKN |
| | | TLYLQMNSLRAEDTAVYYCTIGGSLSPSSQGTLVTVSSGGGGS |
| | | GGGSIWELKKDVYVVELDWYPDAPGEMVVLTCDTPEEDGITW |
| | | TLDQSSEVLGSGKTLTIQVKEFGDAGQYTCHKGGEVLSHSLLL |
| | | LHKKEDGIWSTDILKDQKEPKNKTFLRCEAKNYSGRFTCWWL |
| | | TTISTDLTFSVKSSRGSSDPQGVTCGAATLSAERVRGDNKEYEY |
| | | SVECQEDSACPAAEESLPIEVMVDAVHKLKYENYTSSFFIRDIIK |
| | | PDPPKNLQLKPLKNSRQVEVSWEYPDTWSTPHSYFSLTFCVQV |
| | | QGKSKREKKDRVFTDKTSATVICRKNASISVRAQDRYYSSSWS |
| | | EWASVPCSGGSGGGSGGGSGGGSRNLPVATPDPGMFPCLHHS |
| | | QNLLRAVSNMLQKARQTLEFYPCTSEEIDHEDITKDKTSTVEA |
| | | CLPLELTKNESCLNSRETSFITNGSCLASRKTSFMMALCLSSIYE |
| | | DLKMYQVEFKTMNAKLLMDPKRQIFLDQNMLAVIDELMQAL |
| | | NFNSETVPQKSSLEEPDFYKTKIKLCILLHAFRIRAVTIDRVMSY |
| | | LNASHHHHHHHHHH |
| 54 | 2-LAIR2- | MRVPAQLLGLLLLWLPGARCAAPTSSSTKKTQLQLEHLLLDLQ |
| | ABD-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSQEGALPRPSISAE |
| | | PGTVISPGSHVTFMCRGPVGVQTFRLEREDRAKYKDSYNVFRL |
| | | GPSESEARFHIDSVSEGNAGLYRCLYYKPPGWSEHSDFLELLVK |
| | | ESSGGPDSPDTEPGSSAGTVPGTEASGFDAPGGGGSGGGSKVQ |
| | | LVESGGGLVQPGGSLRLSCAASGFTFSSFGMTWVRQAPGKGLE |
| | | WVSSISGSGSDTLYADSVRGRFTISRDNSKNTLYLQMNSLRAE |
| | | DTAVYYCTIGGSLSPSSQGTLVTVSSGGGGSGGGSIWELKKDV |
| | | YVVELDWYPDAPGEMVVLTCDTPEEDGITWTLDQSSEVLGSG |
| | | KTLTIQVKEFGDAGQYTCHKGGEVLSHSLLLLHKKEDGIWSTD |
| | | ILKDQKEPKNKTFLRCEAKNYSGRFTCWWLTTISTDLTFSVKSS |
| | | RGSSDPQGVTCGAATLSAERVRGDNKEYEYSVECQEDSACPA |
| | | AEESLPIEVMVDAVHKLKYENYTSSFFIRDIIKPDPPKNLQLKPL |
| | | KNSRQVEVSWEYPDTWSTPHSYFSLTFCVQVQGKSKREKKDR |
| | | VFTDKTSATVICRKNASISVRAQDRYYSSSWSEWASVPCSGGS |
| | | GGGSGGGSGGGSRNLPVATPDPGMFPCLHHSQNLLRAVSNML |
| | | QKARQTLEFYPCTSEEIDHEDITKDKTSTVEACLPLELTKNESCL |
| | | NSRETSFITNGSCLASRKTSFMMALCLSSIYEDLKMYQVEFKT |
| | | MNAKLLMDPKRQIFLDQNMLAVIDELMQALNFNSETVPQKSS |
| | | LEEPDFYKTKIKLCILLHAFRIRAVTIDRVMSYLNASHHHHHHH |
| | | HHH |
| 55 | 2-Lum-ABD- | MRVPAQLLGLLLLWLPGARCAAPTSSSTKKTQLQLEHLLLDLQ |
| | 12 (normal | MILNGINNYKNPKLTRMLTFKFYMPKKATELKHLQCLEEELKP |
| | linkers) | LEEVLNLAQSKNFHLRPRDLISNINVIVLELKGSETTFMCEYAD |
| | | ETATIVEFLNRWITFCQSIISTLTGGGGSGGGSSGQYYDYDFPLS |
| | | IYGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSKVQLVESGGGLVQPGGSLRLSCAASGFTFSSFG |
| | | MTWVRQAPGKGLEWVSSISGSGSDTLYADSVRGRFTISRDNSK |
| | | NTLYLQMNSLRAEDTAVYYCTIGGSLSPSSQGTLVTVSSGGGG |
| | | SGGGSIWELKKDVYVVELDWYPDAPGEMVVLTCDTPEEDGIT |
| | | WTLDQSSEVLGSGKTLTIQVKEFGDAGQYTCHKGGEVLSHSLL |
| | | LLHKKEDGIWSTDILKDQKEPKNKTFLRCEAKNYSGRFTCWW |
| | | LTTISTDLTFSVKSSRGSSDPQGVTCGAATLSAERVRGDNKEYE |
| | | YSVECQEDSACPAAEESLPIEVMVDAVHKLKYENYTSSFFIRDII |
| | | KPDPPKNLQLKPLKNSRQVEVSWEYPDTWSTPHSYFSLTFCVQ |
| | | VQGKSKREKKDRVFTDKTSATVICRKNASISVRAQDRYYSSSW |
| | | SEWASVPCSGGSGGGSGGGSGGGSRNLPVATPDPGMFPCLHHS |
| | | QNLLRAVSNMLQKARQTLEFYPCTSEEIDHEDITKDKTSTVEA |
| | | CLPLELTKNESCLNSRETSFITNGSCLASRKTSFMMALCLSSIYE |
| | | DLKMYQVEFKTMNAKLLMDPKRQIFLDQNMLAVIDELMQAL |
| | | NFNSETVPQKSSLEEPDFYKTKIKLCILLHAFRIRAVTIDRVMSY |
| | | LNASHHHHHHHHHH |
| 56 | 2-HSA- | MRVPAQLLGLLLLWLPGARCAAPTSSSTKKTQLQLEHLLLDLQ |
| | LAIR1-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSDAHKSEVAHRF |
| | | KDLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETFLKKY |
| | | LYEIARRHPYFYAPELLFFAKRYKAAFTECCQAADKAACLLPK |
| | | LDELRDEGKASSAKQRLKCASLQKFGERAFKAWAVARLSQRF |
| | | PKAEFAEVSKLVTDLTKVHTECCHGDLLECADDRADLAKYICE |
| | | NQDSISSKLKECCEKPLLEKSHCIAEVENDEMPADLPSLAADFV |
| | | ESKDVCKNYAEAKDVFLGMFLYEYARRHPDYSVVLLLRLAKT |
| | | YETTLEKCCAAADPHECYAKVFDEFKPLVEEPQNLIKQNCELF |
| | | EQLGEYKFQNALLVRYTKKVPQVSTPTLVEVSRNLGKVGSKC |
| | | CKHPEAKRMPCAEDYLSVVLNQLCVLHEKTPVSDRVTKCCTE |
| | | SLVNRRPCFSALEVDETYVPKEFNAETFTFHADICTLSEKERQIK |
| | | KQTALVELVKHKPKATKEQLKAVMDDFAAFVEKCCKADDKE |
| | | TCFAEEGKKLVAASQAALGLGGGGSGGGSQEEDLPRPSISAEP |
| | | GTVIPLGSHVTFVCRGPVGVQTFRLERESRSTYNDTEDVSQASP |
| | | SESEARFRIDSVSEGNAGPYRCIYYKPPKWSEQSDYLELLVKET |
| | | SGGPDSPDTEPGSSAGPTQRPSDNSHNEHAPASQGLKAEHLYG |
| | | GGGSGGGSIWELKKDVYVVELDWYPDAPGEMVVLTCDTPEE |
| | | DGITWTLDQSSEVLGSGKTLTIQVKEFGDAGQYTCHKGGEVLS |
| | | HSLLLLHKKEDGIWSTDILKDQKEPKNKTFLRCEAKNYSGRFT |
| | | CWWLTTISTDLTFSVKSSRGSSDPQGVTCGAATLSAERVRGDN |
| | | KEYEYSVECQEDSACPAAEESLPIEVMVDAVHKLKYENYTSSF |
| | | FIRDIIKPDPPKNLQLKPLKNSRQVEVSWEYPDTWSTPHSYFSLT |
| | | FCVQVQGKSKREKKDRVFTDKTSATVICRKNASISVRAQDRYY |
| | | SSSWSEWASVPCSGGSGGGSGGGSGGGSRNLPVATPDPGMFPC |
| | | LHHSQNLLRAVSNMLQKARQTLEFYPCTSEEIDHEDITKDKTST |
| | | VEACLPLELTKNESCLNSRETSFITNGSCLASRKTSFMMALCLS |
| | | SIYEDLKMYQVEFKTMNAKLLMDPKRQIFLDQNMLAVIDELM |
| | | QALNFNSETVPQKSSLEEPDFYKTKIKLCILLHAFRIRAVTIDRV |
| | | MSYLNASHHHHHHHHHH |
| 57 | 2-HSA- | MRVPAQLLGLLLLWLPGARCAAPTSSSTKKTQLQLEHLLLDLQ |
| | LAIR2-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSDAHKSEVAHRF |
| | | KDLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETFLKKY |
| | | LYEIARRHPYFYAPELLFFAKRYKAAFTECCQAADKAACLLPK |
| | | LDELRDEGKASSAKQRLKCASLQKFGERAFKAWAVARLSQRF |
| | | PKAEFAEVSKLVTDLTKVHTECCHGDLLECADDRADLAKYICE |
| | | NQDSISSKLKECCEKPLLEKSHCIAEVENDEMPADLPSLAADFV |
| | | ESKDVCKNYAEAKDVFLGMFLYEYARRHPDYSVVLLLRLAKT |
| | | YETTLEKCCAAADPHECYAKVFDEFKPLVEEPQNLIKQNCELF |
| | | EQLGEYKFQNALLVRYTKKVPQVSTPTLVEVSRNLGKVGSKC |
| | | CKHPEAKRMPCAEDYLSVVLNQLCVLHEKTPVSDRVTKCCTE |
| | | SLVNRRPCFSALEVDETYVPKEFNAETFTFHADICTLSEKERQIK |
| | | KQTALVELVKHKPKATKEQLKAVMDDFAAFVEKCCKADDKE |
| | | TCFAEEGKKLVAASQAALGLGGGGSGGGSQEGALPRPSISAEP |
| | | GTVISPGSHVTFMCRGPVGVQTFRLEREDRAKYKDSYNVFRLG |
| | | PSESEARFHIDSVSEGNAGLYRCLYYKPPGWSEHSDFLELLVKE |
| | | SSGGPDSPDTEPGSSAGTVPGTEASGFDAPGGGGSGGGSIWELK |
| | | KDVYVVELDWYPDAPGEMVVLTCDTPEEDGITWTLDQSSEVL |
| | | GSGKTLTIQVKEFGDAGQYTCHKGGEVLSHSLLLLHKKEDGIW |
| | | STDILKDQKEPKNKTFLRCEAKNYSGRFTCWWLTTISTDLTFSV |
| | | KSSRGSSDPQGVTCGAATLSAERVRGDNKEYEYSVECQEDSAC |
| | | PAAEESLPIEVMVDAVHKLKYENYTSSFFIRDIIKPDPPKNLQLK |
| | | PLKNSRQVEVSWEYPDTWSTPHSYFSLTFCVQVQGKSKREKK |
| | | DRVFTDKTSATVICRKNASISVRAQDRYYSSSWSEWASVPCSG |
| | | GSGGGSGGGSGGGSRNLPVATPDPGMFPCLHHSQNLLRAVSN |
| | | MLQKARQTLEFYPCTSEEIDHEDITKDKTSTVEACLPLELTKNE |
| | | SCLNSRETSFITNGSCLASRKTSFMMALCLSSIYEDLKMYQVEF |
| | | KTMNAKLLMDPKRQIFLDQNMLAVIDELMQALNFNSETVPQK |
| | | SSLEEPDFYKTKIKLCILLHAFRIRAVTIDRVMSYLNASHHHHH |
| | | HHHHH |
| 58 | 2-HSA-Lum- | MRVPAQLLGLLLLWLPGARCAAPTSSSTKKTQLQLEHLLLDLQ |
| | 12 (normal | MILNGINNYKNPKLTRMLTFKFYMPKKATELKHLQCLEEELKP |
| | linkers) | LEEVLNLAQSKNFHLRPRDLISNINVIVLELKGSETTFMCEYAD |
| | | ETATIVEFLNRWITFCQSIISTLTGGGGSGGGSDAHKSEVAHRF |
| | | KDLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETFLKKY |
| | | LYEIARRHPYFYAPELLFFAKRYKAAFTECCQAADKAACLLPK |
| | | LDELRDEGKASSAKQRLKCASLQKFGERAFKAWAVARLSQRF |
| | | PKAEFAEVSKLVTDLTKVHTECCHGDLLECADDRADLAKYICE |
| | | NQDSISSKLKECCEKPLLEKSHCIAEVENDEMPADLPSLAADFV |
| | | ESKDVCKNYAEAKDVFLGMFLYEYARRHPDYSVVLLLRLAKT |
| | | YETTLEKCCAAADPHECYAKVFDEFKPLVEEPQNLIKQNCELF |
| | | EQLGEYKFQNALLVRYTKKVPQVSTPTLVEVSRNLGKVGSKC |
| | | CKHPEAKRMPCAEDYLSVVLNQLCVLHEKTPVSDRVTKCCTE |
| | | SLVNRRPCFSALEVDETYVPKEFNAETFTFHADICTLSEKERQIK |
| | | KQTALVELVKHKPKATKEQLKAVMDDFAAFVEKCCKADDKE |
| | | TCFAEEGKKLVAASQAALGLGGGGSGGGSSGQYYDYDFPLSIY |
| | | GQSSPNCAPECNCPESYPSAMYCDELKLKSVPMVPPGIKYLYL |
| | | RNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSIWELKKDVYVVELDWYPDAPGEMVVLTCDTP |
| | | EEDGITWTLDQSSEVLGSGKTLTIQVKEFGDAGQYTCHKGGEV |
| | | LSHSLLLLHKKEDGIWSTDILKDQKEPKNKTFLRCEAKNYSGR |
| | | FTCWWLTTISTDLTFSVKSSRGSSDPQGVTCGAATLSAERVRG |
| | | DNKEYEYSVECQEDSACPAAEESLPIEVMVDAVHKLKYENYTS |
| | | SFFIRDIIKPDPPKNLQLKPLKNSRQVEVSWEYPDTWSTPHSYFS |
| | | LTFCVQVQGKSKREKKDRVFTDKTSATVICRKNASISVRAQDR |
| | | YYSSSWSEWASVPCSGGSGGGSGGGSGGGSRNLPVATPDPGM |
| | | FPCLHHSQNLLRAVSNMLQKARQTLEFYPCTSEEIDHEDITKDK |
| | | TSTVEACLPLELTKNESCLNSRETSFITNGSCLASRKTSFMMAL |
| | | CLSSIYEDLKMYQVEFKTMNAKLLMDPKRQIFLDQNMLAVIDE |
| | | LMQALNFNSETVPQKSSLEEPDFYKTKIKLCILLHAFRIRAVTID |
| | | RVMSYLNASHHHHHHHHHH |
| 59 | 2-ABD- | MRVPAQLLGLLLLWLPGARCAAPTSSSTKKTQLQLEHLLLDLQ |
| | LAIR1-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSKVQLVESGGGL |
| | | VQPGGSLRLSCAASGFTFSSFGMTWVRQAPGKGLEWVSSISGS |
| | | GSDTLYADSVRGRFTISRDNSKNTLYLQMNSLRAEDTAVYYCT |
| | | IGGSLSPSSQGTLVTVSSGGGGSGGGSQEEDLPRPSISAEPGTVIP |
| | | LGSHVTFVCRGPVGVQTFRLERESRSTYNDTEDVSQASPSESEA |
| | | RFRIDSVSEGNAGPYRCIYYKPPKWSEQSDYLELLVKETSGGPD |
| | | SPDTEPGSSAGPTQRPSDNSHNEHAPASQGLKAEHLYGGGGSG |
| | | GGSIWELKKDVYVVELDWYPDAPGEMVVLTCDTPEEDGITWT |
| | | LDQSSEVLGSGKTLTIQVKEFGDAGQYTCHKGGEVLSHSLLLL |
| | | HKKEDGIWSTDILKDQKEPKNKTFLRCEAKNYSGRFTCWWLT |
| | | TISTDLTFSVKSSRGSSDPQGVTCGAATLSAERVRGDNKEYEYS |
| | | VECQEDSACPAAEESLPIEVMVDAVHKLKYENYTSSFFIRDIIKP |
| | | DPPKNLQLKPLKNSRQVEVSWEYPDTWSTPHSYFSLTFCVQVQ |
| | | GKSKREKKDRVFTDKTSATVICRKNASISVRAQDRYYSSSWSE |
| | | WASVPCSGGSGGGSGGGSGGGSRNLPVATPDPGMFPCLHHSQ |
| | | NLLRAVSNMLQKARQTLEFYPCTSEEIDHEDITKDKTSTVEACL |
| | | PLELTKNESCLNSRETSFITNGSCLASRKTSFMMALCLSSIYEDL |
| | | KMYQVEFKTMNAKLLMDPKRQIFLDQNMLAVIDELMQALNF |
| | | NSETVPQKSSLEEPDFYKTKIKLCILLHAFRIRAVTIDRVMSYLN |
| | | ASHHHHHHHHHH |
| 60 | 2-ABD- | MRVPAQLLGLLLLWLPGARCAAPTSSSTKKTQLQLEHLLLDLQ |
| | LAIR2-12 | MILNGINNYKNPKLTRMLTFKFYMPKKATELKHLQCLEEELKP |
| | (normal | LEEVLNLAQSKNFHLRPRDLISNINVIVLELKGSETTFMCEYAD |
| | linkers) | ETATIVEFLNRWITFCQSIISTLTGGGGSGGGSKVQLVESGGGL |
| | | VQPGGSLRLSCAASGFTFSSFGMTWVRQAPGKGLEWVSSISGS |
| | | GSDTLYADSVRGRFTISRDNSKNTLYLQMNSLRAEDTAVYYCT |
| | | IGGSLSPSSQGTLVTVSSGGGGSGGGSQEGALPRPSISAEPGTVI |
| | | SPGSHVTFMCRGPVGVQTFRLEREDRAKYKDSYNVFRLGPSES |
| | | EARFHIDSVSEGNAGLYRCLYYKPPGWSEHSDFLELLVKESSG |
| | | GPDSPDTEPGSSAGTVPGTEASGFDAPGGGGSGGGSIWELKKD |
| | | VYVVELDWYPDAPGEMVVLTCDTPEEDGITWTLDQSSEVLGS |
| | | GKTLTIQVKEFGDAGQYTCHKGGEVLSHSLLLLHKKEDGIWST |
| | | DILKDQKEPKNKTFLRCEAKNYSGRFTCWWLTTISTDLTFSVK |
| | | SSRGSSDPQGVTCGAATLSAERVRGDNKEYEYSVECQEDSACP |
| | | AAEESLPIEVMVDAVHKLKYENYTSSFFIRDIIKPDPPKNLQLKP |
| | | LKNSRQVEVSWEYPDTWSTPHSYFSLTFCVQVQGKSKREKKD |
| | | RVFTDKTSATVICRKNASISVRAQDRYYSSSWSEWASVPCSGG |
| | | SGGGSGGGSGGGSRNLPVATPDPGMFPCLHHSQNLLRAVSNM |
| | | LQKARQTLEFYPCTSEEIDHEDITKDKTSTVEACLPLELTKNESC |
| | | LNSRETSFITNGSCLASRKTSFMMALCLSSIYEDLKMYQVEFKT |
| | | MNAKLLMDPKRQIFLDQNMLAVIDELMQALNFNSETVPQKSS |
| | | LEEPDFYKTKIKLCILLHAFRIRAVTIDRVMSYLNASHHHHHHH |
| | | HHH |
| 61 | 2-ABD-Lum- | MRVPAQLLGLLLLWLPGARCAAPTSSSTKKTQLQLEHLLLDLQ |
| | 12 (normal | MILNGINNYKNPKLTRMLTFKFYMPKKATELKHLQCLEEELKP |
| | linkers) | LEEVLNLAQSKNFHLRPRDLISNINVIVLELKGSETTFMCEYAD |
| | | ETATIVEFLNRWITFCQSIISTLTGGGGSGGGSKVQLVESGGGL |
| | | VQPGGSLRLSCAASGFTFSSFGMTWVRQAPGKGLEWVSSISGS |
| | | GSDTLYADSVRGRFTISRDNSKNTLYLQMNSLRAEDTAVYYCT |
| | | IGGSLSPSSQGTLVTVSSGGGGSGGGSSGQYYDYDFPLSIYGQS |
| | | SPNCAPECNCPESYPSAMYCDELKLKSVPMVPPGIKYLYLRNN |
| | | QIDHIDEKAFENVTDLQWLILDHNLLENSKIKGRVFSKLKQLKK |
| | | LHINHNNLTESVGPLPKSLEDLQLTHNKITKLGSFEGLVNLTFIH |
| | | LQHNRLKEDAVSAAFKGLKSLEYLDLSFNQIARLPSGLPVSLLT |
| | | LYLDNNKISNIPDEYFKRFNALQYLRLSHNELADSGIPGNSFNV |
| | | SSLVELDLSYNKLKNIPTVNENLENYYLEVNQLEKFDIKSFCKI |
| | | LGPLSYSKIKHLRLDGNRISETSLPPDMYECLRVANEVTLNGGG |
| | | GSGGGSIWELKKDVYVVELDWYPDAPGEMVVLTCDTPEEDGI |
| | | TWTLDQSSEVLGSGKTLTIQVKEFGDAGQYTCHKGGEVLSHSL |
| | | LLLHKKEDGIWSTDILKDQKEPKNKTFLRCEAKNYSGRFTCW |
| | | WLTTISTDLTFSVKSSRGSSDPQGVTCGAATLSAERVRGDNKE |
| | | YEYSVECQEDSACPAAEESLPIEVMVDAVHKLKYENYTSSFFIR |
| | | DIIKPDPPKNLQLKPLKNSRQVEVSWEYPDTWSTPHSYFSLTFC |
| | | VQVQGKSKREKKDRVFTDKTSATVICRKNASISVRAQDRYYSS |
| | | SWSEWASVPCSGGSGGGSGGGSGGGSRNLPVATPDPGMFPCL |
| | | HHSQNLLRAVSNMLQKARQTLEFYPCTSEEIDHEDITKDKTST |
| | | VEACLPLELTKNESCLNSRETSFITNGSCLASRKTSFMMALCLS |
| | | SIYEDLKMYQVEFKTMNAKLLMDPKRQIFLDQNMLAVIDELM |
| | | QALNFNSETVPQKSSLEEPDFYKTKIKLCILLHAFRIRAVTIDRV |
| | | MSYLNASHHHHHHHHHH |
| 62 | 12-LAIR1- | MRVPAQLLGLLLLWLPGARCAIWELKKDVYVVELDWYPDAP |
| | HSA H464Q- | GEMVVLTCDTPEEDGITWTLDQSSEVLGSGKTLTIQVKEFGDA |
| | 2 (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSQEEDLPRPSISAE |
| | | PGTVIPLGSHVTFVCRGPVGVQTFRLERESRSTYNDTEDVSQAS |
| | | PSESEARFRIDSVSEGNAGPYRCIYYKPPKWSEQSDYLELLVKE |
| | | TSGGPDSPDTEPGSSAGPTQRPSDNSHNEHAPASQGLKAEHLY |
| | | GGGGSGGGSDAHKSEVAHRFKDLGEENFKALVLIAFAQYLQQ |
| | | CPFEDHVKLVNEVTEFAKTCVADESAENCDKSLHTLFGDKLCT |
| | | VATLRETYGEMADCCAKQEPERNECFLQHKDDNPNLPRLVRP |
| | | EVDVMCTAFHDNEETFLKKYLYEIARRHPYFYAPELLFFAKRY |
| | | KAAFTECCQAADKAACLLPKLDELRDEGKASSAKQRLKCASL |
| | | QKFGERAFKAWAVARLSQRFPKAEFAEVSKLVTDLTKVHTEC |
| | | CHGDLLECADDRADLAKYICENQDSISSKLKECCEKPLLEKSH |
| | | CIAEVENDEMPADLPSLAADFVESKDVCKNYAEAKDVFLGMF |
| | | LYEYARRHPDYSVVLLLRLAKTYETTLEKCCAAADPHECYAK |
| | | VFDEFKPLVEEPQNLIKQNCELFEQLGEYKFQNALLVRYTKKV |
| | | PQVSTPTLVEVSRNLGKVGSKCCKHPEAKRMPCAEDYLSVVL |
| | | NQLCVLQEKTPVSDRVTKCCTESLVNRRPCFSALEVDETYVPK |
| | | EFNAETFTFHADICTLSEKERQIKKQTALVELVKHKPKATKEQL |
| | | KAVMDDFAAFVEKCCKADDKETCFAEEGKKLVAASQAALGL |
| | | GGGGSGGGSAPTSSSTKKTQLQLEHLLLDLQMILNGINNYKNP |
| | | KLTRMLTFKFYMPKKATELKHLQCLEEELKPLEEVLNLAQSKN |
| | | FHLRPRDLISNINVIVLELKGSETTFMCEYADETATIVEFLNRWI |
| | | TFCQSIISTLTHHHHHHHHHH |
| 63 | 12-LAIR2- | MRVPAQLLGLLLLWLPGARCAIWELKKDVYVVELDWYPDAP |
| | HSA H464Q | GEMVVLTCDTPEEDGITWTLDQSSEVLGSGKTLTIQVKEFGDA |
| | -2 (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSQEGALPRPSISAE |
| | | PGTVISPGSHVTFMCRGPVGVQTFRLEREDRAKYKDSYNVFRL |
| | | GPSESEARFHIDSVSEGNAGLYRCLYYKPPGWSEHSDFLELLVK |
| | | ESSGGPDSPDTEPGSSAGTVPGTEASGFDAPGGGGSGGGSDAH |
| | | KSEVAHRFKDLGEENFKALVLIAFAQYLQQCPFEDHVKLVNEV |
| | | TEFAKTCVADESAENCDKSLHTLFGDKLCTVATLRETYGEMA |
| | | DCCAKQEPERNECFLQHKDDNPNLPRLVRPEVDVMCTAFHDN |
| | | EETFLKKYLYEIARRHPYFYAPELLFFAKRYKAAFTECCQAAD |
| | | KAACLLPKLDELRDEGKASSAKQRLKCASLQKFGERAFKAWA |
| | | VARLSQRFPKAEFAEVSKLVTDLTKVHTECCHGDLLECADDR |
| | | ADLAKYICENQDSISSKLKECCEKPLLEKSHCIAEVENDEMPAD |
| | | LPSLAADFVESKDVCKNYAEAKDVFLGMFLYEYARRHPDYSV |
| | | VLLLRLAKTYETTLEKCCAAADPHECYAKVFDEFKPLVEEPQN |
| | | LIKQNCELFEQLGEYKFQNALLVRYTKKVPQVSTPTLVEVSRN |
| | | LGKVGSKCCKHPEAKRMPCAEDYLSVVLNQLCVLQEKTPVSD |
| | | RVTKCCTESLVNRRPCFSALEVDETYVPKEFNAETFTFHADICT |
| | | LSEKERQIKKQTALVELVKHKPKATKEQLKAVMDDFAAFVEK |
| | | CCKADDKETCFAEEGKKLVAASQAALGLGGGGSGGGSAPTSS |
| | | STKKTQLQLEHLLLDLQMILNGINNYKNPKLTRMLTFKFYMPK |
| | | KATELKHLQCLEEELKPLEEVLNLAQSKNFHLRPRDLISNINVI |
| | | VLELKGSETTFMCEYADETATIVEFLNRWITFCQSIISTLTHHHH |
| | | HHHHHH |
| 64 | 12-Lum- | MRVPAQLLGLLLLWLPGARCAIWELKKDVYVVELDWYPDAP |
| | HSA_H464Q- | GEMVVLTCDTPEEDGITWTLDQSSEVLGSGKTLTIQVKEFGDA |
| | 2 (normal | GQYTCHKGGEVLSHSLLLLHKKEDGIWSTDILKDQKEPKNKTF |
| | linkers) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGGSGGGSSGQYYDYDFPLS |
| | | IYGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSDAHKSEVAHRFKDLGEENFKALVLIAFAQYLQ |
| | | QCPFEDHVKLVNEVTEFAKTCVADESAENCDKSLHTLFGDKLC |
| | | TVATLRETYGEMADCCAKQEPERNECFLQHKDDNPNLPRLVR |
| | | PEVDVMCTAFHDNEETFLKKYLYEIARRHPYFYAPELLFFAKR |
| | | YKAAFTECCQAADKAACLLPKLDELRDEGKASSAKQRLKCAS |
| | | LQKFGERAFKAWAVARLSQRFPKAEFAEVSKLVTDLTKVHTE |
| | | CCHGDLLECADDRADLAKYICENQDSISSKLKECCEKPLLEKS |
| | | HCIAEVENDEMPADLPSLAADFVESKDVCKNYAEAKDVFLGM |
| | | FLYEYARRHPDYSVVLLLRLAKTYETTLEKCCAAADPHECYA |
| | | KVFDEFKPLVEEPQNLIKQNCELFEQLGEYKFQNALLVRYTKK |
| | | VPQVSTPTLVEVSRNLGKVGSKCCKHPEAKRMPCAEDYLSVV |
| | | LNQLCVLQEKTPVSDRVTKCCTESLVNRRPCFSALEVDETYVP |
| | | KEFNAETFTFHADICTLSEKERQIKKQTALVELVKHKPKATKEQ |
| | | LKAVMDDFAAFVEKCCKADDKETCFAEEGKKLVAASQAALG |
| | | LGGGGSGGGSAPTSSSTKKTQLQLEHLLLDLQMILNGINNYKN |
| | | PKLTRMLTFKFYMPKKATELKHLQCLEEELKPLEEVLNLAQSK |
| | | NFHLRPRDLISNINVIVLELKGSETTFMCEYADETATIVEFLNR |
| | | WITFCQSIISTLTHHHHHHHHHH |
| 65 | 2-LAIR1- | MRVPAQLLGLLLLWLPGARCAAPTSSSTKKTQLQLEHLLLDLQ |
| | HSA H464Q- | MILNGINNYKNPKLTRMLTFKFYMPKKATELKHLQCLEEELKP |
| | 12 (normal | LEEVLNLAQSKNFHLRPRDLISNINVIVLELKGSETTFMCEYAD |
| | linkers) | ETATIVEFLNRWITFCQSIISTLTGGGGSGGGSQEEDLPRPSISAE |
| | | PGTVIPLGSHVTFVCRGPVGVQTFRLERESRSTYNDTEDVSQAS |
| | | PSESEARFRIDSVSEGNAGPYRCIYYKPPKWSEQSDYLELLVKE |
| | | TSGGPDSPDTEPGSSAGPTQRPSDNSHNEHAPASQGLKAEHLY |
| | | GGGGSGGGSDAHKSEVAHRFKDLGEENFKALVLIAFAQYLQQ |
| | | CPFEDHVKLVNEVTEFAKTCVADESAENCDKSLHTLFGDKLCT |
| | | VATLRETYGEMADCCAKQEPERNECFLQHKDDNPNLPRLVRP |
| | | EVDVMCTAFHDNEETFLKKYLYEIARRHPYFYAPELLFFAKRY |
| | | KAAFTECCQAADKAACLLPKLDELRDEGKASSAKQRLKCASL |
| | | QKFGERAFKAWAVARLSQRFPKAEFAEVSKLVTDLTKVHTEC |
| | | CHGDLLECADDRADLAKYICENQDSISSKLKECCEKPLLEKSH |
| | | CIAEVENDEMPADLPSLAADFVESKDVCKNYAEAKDVFLGMF |
| | | LYEYARRHPDYSVVLLLRLAKTYETTLEKCCAAADPHECYAK |
| | | VFDEFKPLVEEPQNLIKQNCELFEQLGEYKFQNALLVRYTKKV |
| | | PQVSTPTLVEVSRNLGKVGSKCCKHPEAKRMPCAEDYLSVVL |
| | | NQLCVLQEKTPVSDRVTKCCTESLVNRRPCFSALEVDETYVPK |
| | | EFNAETFTFHADICTLSEKERQIKKQTALVELVKHKPKATKEQL |
| | | KAVMDDFAAFVEKCCKADDKETCFAEEGKKLVAASQAALGL |
| | | GGGGSGGGSIWELKKDVYVVELDWYPDAPGEMVVLTCDTPE |
| | | EDGITWTLDQSSEVLGSGKTLTIQVKEFGDAGQYTCHKGGEVL |
| | | SHSLLLLHKKEDGIWSTDILKDQKEPKNKTFLRCEAKNYSGRF |
| | | TCWWLTTISTDLTFSVKSSRGSSDPQGVTCGAATLSAERVRGD |
| | | NKEYEYSVECQEDSACPAAEESLPIEVMVDAVHKLKYENYTSS |
| | | FFIRDIIKPDPPKNLQLKPLKNSRQVEVSWEYPDTWSTPHSYFSL |
| | | TFCVQVQGKSKREKKDRVFTDKTSATVICRKNASISVRAQDRY |
| | | YSSSWSEWASVPCSGGSGGGSGGGSGGGSRNLPVATPDPGMF |
| | | PCLHHSQNLLRAVSNMLQKARQTLEFYPCTSEEIDHEDITKDKT |
| | | STVEACLPLELTKNESCLNSRETSFITNGSCLASRKTSFMMALC |
| | | LSSIYEDLKMYQVEFKTMNAKLLMDPKRQIFLDQNMLAVIDEL |
| | | MQALNFNSETVPQKSSLEEPDFYKTKIKLCILLHAFRIRAVTIDR |
| | | VMSYLNASHHHHHHHHHH |
| 66 | 2-LAIR2- | MRVPAQLLGLLLLWLPGARCAAPTSSSTKKTQLQLEHLLLDLQ |
| | HSA_H464Q | MILNGINNYKNPKLTRMLTFKFYMPKKATELKHLQCLEEELKP |
| | -12 (normal | LEEVLNLAQSKNFHLRPRDLISNINVIVLELKGSETTFMCEYAD |
| | linkers) | ETATIVEFLNRWITFCQSIISTLTGGGGSGGGSQEGALPRPSISAE |
| | | PGTVISPGSHVTFMCRGPVGVQTFRLEREDRAKYKDSYNVFRL |
| | | GPSESEARFHIDSVSEGNAGLYRCLYYKPPGWSEHSDFLELLVK |
| | | ESSGGPDSPDTEPGSSAGTVPGTEASGFDAPGGGGSGGGSDAH |
| | | KSEVAHRFKDLGEENFKALVLIAFAQYLQQCPFEDHVKLVNEV |
| | | TEFAKTCVADESAENCDKSLHTLFGDKLCTVATLRETYGEMA |
| | | DCCAKQEPERNECFLQHKDDNPNLPRLVRPEVDVMCTAFHDN |
| | | EETFLKKYLYEIARRHPYFYAPELLFFAKRYKAAFTECCQAAD |
| | | KAACLLPKLDELRDEGKASSAKQRLKCASLQKFGERAFKAWA |
| | | VARLSQRFPKAEFAEVSKLVTDLTKVHTECCHGDLLECADDR |
| | | ADLAKYICENQDSISSKLKECCEKPLLEKSHCIAEVENDEMPAD |
| | | LPSLAADFVESKDVCKNYAEAKDVFLGMFLYEYARRHPDYSV |
| | | VLLLRLAKTYETTLEKCCAAADPHECYAKVFDEFKPLVEEPQN |
| | | LIKQNCELFEQLGEYKFQNALLVRYTKKVPQVSTPTLVEVSRN |
| | | LGKVGSKCCKHPEAKRMPCAEDYLSVVLNQLCVLQEKTPVSD |
| | | RVTKCCTESLVNRRPCFSALEVDETYVPKEFNAETFTFHADICT |
| | | LSEKERQIKKQTALVELVKHKPKATKEQLKAVMDDFAAFVEK |
| | | CCKADDKETCFAEEGKKLVAASQAALGLGGGGSGGGSIWELK |
| | | KDVYVVELDWYPDAPGEMVVLTCDTPEEDGITWTLDQSSEVL |
| | | GSGKTLTIQVKEFGDAGQYTCHKGGEVLSHSLLLLHKKEDGIW |
| | | STDILKDQKEPKNKTFLRCEAKNYSGRFTCWWLTTISTDLTFSV |
| | | KSSRGSSDPQGVTCGAATLSAERVRGDNKEYEYSVECQEDSAC |
| | | PAAEESLPIEVMVDAVHKLKYENYTSSFFIRDIIKPDPPKNLQLK |
| | | PLKNSRQVEVSWEYPDTWSTPHSYFSLTFCVQVQGKSKREKK |
| | | DRVFTDKTSATVICRKNASISVRAQDRYYSSSWSEWASVPCSG |
| | | GSGGGSGGGSGGGSRNLPVATPDPGMFPCLHHSQNLLRAVSN |
| | | MLQKARQTLEFYPCTSEEIDHEDITKDKTSTVEACLPLELTKNE |
| | | SCLNSRETSFITNGSCLASRKTSFMMALCLSSIYEDLKMYQVEF |
| | | KTMNAKLLMDPKRQIFLDQNMLAVIDELMQALNFNSETVPQK |
| | | SSLEEPDFYKTKIKLCILLHAFRIRAVTIDRVMSYLNASHHHHH |
| | | HHHHH |
| 67 | 2-Lum- | MRVPAQLLGLLLLWLPGARCAAPTSSSTKKTQLQLEHLLLDLQ |
| | HSA H464Q- | MILNGINNYKNPKLTRMLTFKFYMPKKATELKHLQCLEEELKP |
| | 12 (normal | LEEVLNLAQSKNFHLRPRDLISNINVIVLELKGSETTFMCEYAD |
| | linkers) | ETATIVEFLNRWITFCQSIISTLTGGGGSGGGSSGQYYDYDFPLS |
| | | IYGQSSPNCAPECNCPESYPSAMYCDELKLKSVPMVPPGIKYLY |
| | | LRNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSDAHKSEVAHRFKDLGEENFKALVLIAFAQYLQ |
| | | QCPFEDHVKLVNEVTEFAKTCVADESAENCDKSLHTLFGDKLC |
| | | TVATLRETYGEMADCCAKQEPERNECFLQHKDDNPNLPRLVR |
| | | PEVDVMCTAFHDNEETFLKKYLYEIARRHPYFYAPELLFFAKR |
| | | YKAAFTECCQAADKAACLLPKLDELRDEGKASSAKQRLKCAS |
| | | LQKFGERAFKAWAVARLSQRFPKAEFAEVSKLVTDLTKVHTE |
| | | CCHGDLLECADDRADLAKYICENQDSISSKLKECCEKPLLEKS |
| | | HCIAEVENDEMPADLPSLAADFVESKDVCKNYAEAKDVFLGM |
| | | FLYEYARRHPDYSVVLLLRLAKTYETTLEKCCAAADPHECYA |
| | | KVFDEFKPLVEEPQNLIKQNCELFEQLGEYKFQNALLVRYTKK |
| | | VPQVSTPTLVEVSRNLGKVGSKCCKHPEAKRMPCAEDYLSVV |
| | | LNQLCVLQEKTPVSDRVTKCCTESLVNRRPCFSALEVDETYVP |
| | | KEFNAETFTFHADICTLSEKERQIKKQTALVELVKHKPKATKEQ |
| | | LKAVMDDFAAFVEKCCKADDKETCFAEEGKKLVAASQAALG |
| | | LGGGGSGGGSIWELKKDVYVVELDWYPDAPGEMVVLTCDTP |
| | | EEDGITWTLDQSSEVLGSGKTLTIQVKEFGDAGQYTCHKGGEV |
| | | LSHSLLLLHKKEDGIWSTDILKDQKEPKNKTFLRCEAKNYSGR |
| | | FTCWWLTTISTDLTFSVKSSRGSSDPQGVTCGAATLSAERVRG |
| | | DNKEYEYSVECQEDSACPAAEESLPIEVMVDAVHKLKYENYTS |
| | | SFFIRDIIKPDPPKNLQLKPLKNSRQVEVSWEYPDTWSTPHSYFS |
| | | LTFCVQVQGKSKREKKDRVFTDKTSATVICRKNASISVRAQDR |
| | | YYSSSWSEWASVPCSGGSGGGSGGGSGGGSRNLPVATPDPGM |
| | | FPCLHHSQNLLRAVSNMLQKARQTLEFYPCTSEEIDHEDITKDK |
| | | TSTVEACLPLELTKNESCLNSRETSFITNGSCLASRKTSFMMAL |
| | | CLSSIYEDLKMYQVEFKTMNAKLLMDPKRQIFLDQNMLA VIDE |
| | | LMQALNFNSETVPQKSSLEEPDFYKTKIKLCILLHAFRIRAVTID |
| | | RVMSYLNASHHHHHHHHHH |
| 68 | 2- | MRVPAQLLGLLLLWLPGARCAAPTSSSTKKTQLQLEHLLLDLQ |
| | HSA H464Q- | MILNGINNYKNPKLTRMLTFKFYMPKKATELKHLQCLEEELKP |
| | LAIR1-12 | LEEVLNLAQSKNFHLRPRDLISNINVIVLELKGSETTFMCEYAD |
| | (normal | ETATIVEFLNRWITFCQSIISTLTGGGGSGGGSDAHKSEVAHRF |
| | linkers) | KDLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETFLKKY |
| | | LYEIARRHPYFYAPELLFFAKRYKAAFTECCQAADKAACLLPK |
| | | LDELRDEGKASSAKQRLKCASLQKFGERAFKAWAVARLSQRF |
| | | PKAEFAEVSKLVTDLTKVHTECCHGDLLECADDRADLAKYICE |
| | | NQDSISSKLKECCEKPLLEKSHCIAEVENDEMPADLPSLAADFV |
| | | ESKDVCKNYAEAKDVFLGMFLYEYARRHPDYSVVLLLRLAKT |
| | | YETTLEKCCAAADPHECYAKVFDEFKPLVEEPQNLIKQNCELF |
| | | EQLGEYKFQNALLVRYTKKVPQVSTPTLVEVSRNLGKVGSKC |
| | | CKHPEAKRMPCAEDYLSVVLNQLCVLQEKTPVSDRVTKCCTE |
| | | SLVNRRPCFSALEVDETYVPKEFNAETFTFHADICTLSEKERQIK |
| | | KQTALVELVKHKPKATKEQLKAVMDDFAAFVEKCCKADDKE |
| | | TCFAEEGKKLVAASQAALGLGGGGSGGGSQEEDLPRPSISAEP |
| | | GTVIPLGSHVTFVCRGPVGVQTFRLERESRSTYNDTEDVSQASP |
| | | SESEARFRIDSVSEGNAGPYRCIYYKPPKWSEQSDYLELLVKET |
| | | SGGPDSPDTEPGSSAGPTQRPSDNSHNEHAPASQGLKAEHLYG |
| | | GGGSGGGSIWELKKDVYVVELDWYPDAPGEMVVLTCDTPEE |
| | | DGITWTLDQSSEVLGSGKTLTIQVKEFGDAGQYTCHKGGEVLS |
| | | HSLLLLHKKEDGIWSTDILKDQKEPKNKTFLRCEAKNYSGRFT |
| | | CWWLTTISTDLTFSVKSSRGSSDPQGVTCGAATLSAERVRGDN |
| | | KEYEYSVECQEDSACPAAEESLPIEVMVDAVHKLKYENYTSSF |
| | | FIRDIIKPDPPKNLQLKPLKNSRQVEVSWEYPDTWSTPHSYFSLT |
| | | FCVQVQGKSKREKKDRVFTDKTSATVICRKNASISVRAQDRYY |
| | | SSSWSEWASVPCSGGSGGGSGGGSGGGSRNLPVATPDPGMFPC |
| | | LHHSQNLLRAVSNMLQKARQTLEFYPCTSEEIDHEDITKDKTST |
| | | VEACLPLELTKNESCLNSRETSFITNGSCLASRKTSFMMALCLS |
| | | SIYEDLKMYQVEFKTMNAKLLMDPKRQIFLDQNMLAVIDELM |
| | | QALNFNSETVPQKSSLEEPDFYKTKIKLCILLHAFRIRAVTIDRV |
| | | MSYLNASHHHHHHHHHH |
| 69 | 2- | MRVPAQLLGLLLLWLPGARCAAPTSSSTKKTQLQLEHLLLDLQ |
| | HSA_H464Q- | MILNGINNYKNPKLTRMLTFKFYMPKKATELKHLQCLEEELKP |
| | LAIR2-12 | LEEVLNLAQSKNFHLRPRDLISNINVIVLELKGSETTFMCEYAD |
| | (normal | ETATIVEFLNRWITFCQSIISTLTGGGGSGGGSDAHKSEVAHRF |
| | linkers) | KDLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETFLKKY |
| | | LYEIARRHPYFYAPELLFFAKRYKAAFTECCQAADKAACLLPK |
| | | LDELRDEGKASSAKQRLKCASLQKFGERAFKAWAVARLSQRF |
| | | PKAEFAEVSKLVTDLTKVHTECCHGDLLECADDRADLAKYICE |
| | | NQDSISSKLKECCEKPLLEKSHCIAEVENDEMPADLPSLAADFV |
| | | ESKDVCKNYAEAKDVFLGMFLYEYARRHPDYSVVLLLRLAKT |
| | | YETTLEKCCAAADPHECYAKVFDEFKPLVEEPQNLIKQNCELF |
| | | EQLGEYKFQNALLVRYTKKVPQVSTPTLVEVSRNLGKVGSKC |
| | | CKHPEAKRMPCAEDYLSVVLNQLCVLQEKTPVSDRVTKCCTE |
| | | SLVNRRPCFSALEVDETYVPKEFNAETFTFHADICTLSEKERQIK |
| | | KQTALVELVKHKPKATKEQLKAVMDDFAAFVEKCCKADDKE |
| | | TCFAEEGKKLVAASQAALGLGGGGSGGGSQEGALPRPSISAEP |
| | | GTVISPGSHVTFMCRGPVGVQTFRLEREDRAKYKDSYNVFRLG |
| | | PSESEARFHIDSVSEGNAGLYRCLYYKPPGWSEHSDFLELLVKE |
| | | SSGGPDSPDTEPGSSAGTVPGTEASGFDAPGGGGSGGGSIWELK |
| | | KDVYVVELDWYPDAPGEMVVLTCDTPEEDGITWTLDQSSEVL |
| | | GSGKTLTIQVKEFGDAGQYTCHKGGEVLSHSLLLLHKKEDGIW |
| | | STDILKDQKEPKNKTFLRCEAKNYSGRFTCWWLTTISTDLTFSV |
| | | KSSRGSSDPQGVTCGAATLSAERVRGDNKEYEYSVECQEDSAC |
| | | PAAEESLPIEVMVDAVHKLKYENYTSSFFIRDIIKPDPPKNLQLK |
| | | PLKNSRQVEVSWEYPDTWSTPHSYFSLTFCVQVQGKSKREKK |
| | | DRVFTDKTSATVICRKNASISVRAQDRYYSSSWSEWASVPCSG |
| | | GSGGGSGGGSGGGSRNLPVATPDPGMFPCLHHSQNLLRAVSN |
| | | MLQKARQTLEFYPCTSEEIDHEDITKDKTSTVEACLPLELTKNE |
| | | SCLNSRETSFITNGSCLASRKTSFMMALCLSSIYEDLKMYQVEF |
| | | KTMNAKLLMDPKRQIFLDQNMLAVIDELMQALNFNSETVPQK |
| | | SSLEEPDFYKTKIKLCILLHAFRIRAVTIDRVMSYLNASHHHHH |
| | | HHHHH |
| 70 | 2- | MRVPAQLLGLLLLWLPGARCAAPTSSSTKKTQLQLEHLLLDLQ |
| | HSA_H464Q- | MILNGINNYKNPKLTRMLTFKFYMPKKATELKHLQCLEEELKP |
| | Lum-12 | LEEVLNLAQSKNFHLRPRDLISNINVIVLELKGSETTFMCEYAD |
| | (normal | ETATIVEFLNRWITFCQSIISTLTGGGGSGGGSDAHKSEVAHRF |
| | linkers) | KDLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETFLKKY |
| | | LYEIARRHPYFYAPELLFFAKRYKAAFTECCQAADKAACLLPK |
| | | LDELRDEGKASSAKQRLKCASLQKFGERAFKAWAVARLSQRF |
| | | PKAEFAEVSKLVTDLTKVHTECCHGDLLECADDRADLAKYICE |
| | | NQDSISSKLKECCEKPLLEKSHCIAEVENDEMPADLPSLAADFV |
| | | ESKDVCKNYAEAKDVFLGMFLYEYARRHPDYSVVLLLRLAKT |
| | | YETTLEKCCAAADPHECYAKVFDEFKPLVEEPQNLIKQNCELF |
| | | EQLGEYKFQNALLVRYTKKVPQVSTPTLVEVSRNLGKVGSKC |
| | | CKHPEAKRMPCAEDYLSVVLNQLCVLQEKTPVSDRVTKCCTE |
| | | SLVNRRPCFSALEVDETYVPKEFNAETFTFHADICTLSEKERQIK |
| | | KQTALVELVKHKPKATKEQLKAVMDDFAAFVEKCCKADDKE |
| | | TCFAEEGKKLVAASQAALGLGGGGSGGGSSGQYYDYDFPLSIY |
| | | GQSSPNCAPECNCPESYPSAMYCDELKLKSVPMVPPGIKYLYL |
| | | RNNQIDHIDEKAFENVTDLQWLILDHNLLENSKIKGRVFSKLK |
| | | QLKKLHINHNNLTESVGPLPKSLEDLQLTHNKITKLGSFEGLVN |
| | | LTFIHLQHNRLKEDAVSAAFKGLKSLEYLDLSFNQIARLPSGLP |
| | | VSLLTLYLDNNKISNIPDEYFKRFNALQYLRLSHNELADSGIPG |
| | | NSFNVSSLVELDLSYNKLKNIPTVNENLENYYLEVNQLEKFDIK |
| | | SFCKILGPLSYSKIKHLRLDGNRISETSLPPDMYECLRVANEVTL |
| | | NGGGGSGGGSIWELKKDVYVVELDWYPDAPGEMVVLTCDTP |
| | | EEDGITWTLDQSSEVLGSGKTLTIQVKEFGDAGQYTCHKGGEV |
| | | LSHSLLLLHKKEDGIWSTDILKDQKEPKNKTFLRCEAKNYSGR |
| | | FTCWWLTTISTDLTFSVKSSRGSSDPQGVTCGAATLSAERVRG |
| | | DNKEYEYSVECQEDSACPAAEESLPIEVMVDAVHKLKYENYTS |
| | | SFFIRDIIKPDPPKNLQLKPLKNSRQVEVSWEYPDTWSTPHSYFS |
| | | LTFCVQVQGKSKREKKDRVFTDKTSATVICRKNASISVRAQDR |
| | | YYSSSWSEWASVPCSGGSGGGSGGGSGGGSRNLPVATPDPGM |
| | | FPCLHHSQNLLRAVSNMLQKARQTLEFYPCTSEEIDHEDITKDK |
| | | TSTVEACLPLELTKNESCLNSRETSFITNGSCLASRKTSFMMAL |
| | | CLSSIYEDLKMYQVEFKTMNAKLLMDPKRQIFLDQNMLAVIDE |
| | | LMQALNFNSETVPQKSSLEEPDFYKTKIKLCILLHAFRIRAVTID |
| | | RVMSYLNASHHHHHHHHHH |
| 73 | 12-LAIR2- | MRVPAQLLGLLLLWLPGARCAIWELKKDVYVVELDWYPDAP |
| | HSA-2 (no | GEMVVLTCDTPEEDGITWTLDQSSEVLGSGKTLTIQVKEFGDA |
| | Histidine | GQYTCHKGGEVLSHSLLLLHKKEDGIWSTDILKDQKEPKNKTF |
| | Tag) | LRCEAKNYSGRFTCWWLTTISTDLTFSVKSSRGSSDPQGVTCG |
| | | AATLSAERVRGDNKEYEYSVECQEDSACPAAEESLPIEVMVDA |
| | | VHKLKYENYTSSFFIRDIIKPDPPKNLQLKPLKNSRQVEVSWEY |
| | | PDTWSTPHSYFSLTFCVQVQGKSKREKKDRVFTDKTSATVICR |
| | | KNASISVRAQDRYYSSSWSEWASVPCSGGSGGGSGGGSGGGS |
| | | RNLPVATPDPGMFPCLHHSQNLLRAVSNMLQKARQTLEFYPCT |
| | | SEEIDHEDITKDKTSTVEACLPLELTKNESCLNSRETSFITNGSCL |
| | | ASRKTSFMMALCLSSIYEDLKMYQVEFKTMNAKLLMDPKRQI |
| | | FLDQNMLAVIDELMQALNFNSETVPQKSSLEEPDFYKTKIKLCI |
| | | LLHAFRIRAVTIDRVMSYLNASGGGSGGGSQEGALPRPSISAEP |
| | | GTVISPGSHVTFMCRGPVGVQTFRLEREDRAKYKDSYNVFRLG |
| | | PSESEARFHIDSVSEGNAGLYRCLYYKPPGWSEHSDFLELLVKE |
| | | SSGGPDSPDTEPGSSAGTVPGTEASGFDAPGGGSGGGSDAHKS |
| | | EVAHRFKDLGEENFKALVLIAFAQYLQQCPFEDHVKLVNEVTE |
| | | FAKTCVADESAENCDKSLHTLFGDKLCTVATLRETYGEMADC |
| | | CAKQEPERNECFLQHKDDNPNLPRLVRPEVDVMCTAFHDNEE |
| | | TFLKKYLYEIARRHPYFYAPELLFFAKRYKAAFTECCQAADKA |
| | | ACLLPKLDELRDEGKASSAKQRLKCASLQKFGERAFKAWAVA |
| | | RLSQRFPKAEFAEVSKLVTDLTKVHTECCHGDLLECADDRADL |
| | | AKYICENQDSISSKLKECCEKPLLEKSHCIAEVENDEMPADLPS |
| | | LAADFVESKDVCKNYAEAKDVFLGMFLYEYARRHPDYSVVLL |
| | | LRLAKTYETTLEKCCAAADPHECYAKVFDEFKPLVEEPQNLIK |
| | | QNCELFEQLGEYKFQNALLVRYTKKVPQVSTPTLVEVSRNLGK |
| | | VGSKCCKHPEAKRMPCAEDYLSVVLNQLCVLHEKTPVSDRVT |
| | | KCCTESLVNRRPCFSALEVDETYVPKEFNAETFTFHADICTLSE |
| | | KERQIKKQTALVELVKHKPKATKEQLKAVMDDFAAFVEKCCK |
| | | ADDKETCFAEEGKKLVAASQAALGLGGGSAPTSSSTKKTQLQL |
| | | EHLLLDLQMILNGINNYKNPKLTRMLTFKFYMPKKATELKHLQ |
| | | CLEEELKPLEEVLNLAQSKNFHLRPRDLISNINVIVLELKGSETT |
| | | FMCEYADETATIVEFLNRWITFCQSIISTLT |

VI. Methods for Making Immunomodulatory Fusion Proteins

The immunomodulatory fusion proteins of the present invention are made using recombinant DNA technology. In some aspects, the domains of the immunomodulatory fusion proteins described herein (e.g., collagen-binding domains, cytokines) are made in transformed host cells using recombinant DNA techniques. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The immunomodulatory fusion proteins of the present invention are isolated and purified using one or more methods known in the art, including centrifugation, depth filtration, cell lysis, homogenization, freeze thawing, affinity purification, gel filtration, size exchange chromatography, ion exchange chromatography, hydrophobic interaction exchange chromatography, and mixed-mode chromatography. In certain embodiments, the fusion proteins described herein are purified by size exchange chromatography with a protein A resin. In certain embodiments, the fusion proteins described herein are purified by size exchange chromatography with Capto™ Blue resin. In certain embodiments, the fusion proteins described herein are purified by size exchange chromatography with CaptureSelect™ HSA resin. In certain embodiments, the purified fusion proteins described herein are concentrated by any suitable method known in the art. In certain embodiments, the purified fusion protein is concentrated to a concentration of 0.1-100 mg/ml, 1-50 mg/ml, or 10-30 mg/ml. In certain embodiments, the purified fusion protein is concentrated to a concentration of 0.1-100 mg/ml, 1-50 mg/ml, or 10-30 mg/ml without detectable aggregation of the fusion protein. In certain embodiments, the purified fusion protein is concentrated to a concentration of about 20 mg/ml without detectable aggregation of the fusion protein.

In one exemplary embodiment, codon-optimized DNA sequences encoding comprising IL-12, IL-2, a collagen-binding protein, and albumin were synthesized and cloned into a pD2610-v1 vector. Plasmids were transformed into DH10B competent cells for expansion. Purified expression vectors were transiently transfected into HEK293 cells. Recombinant proteins were purified via anion exchange using Q Sepharose resin and preparative size exclusion chromatography (SEC).

VII. Pharmaceutical Compositions and Modes of Administration

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975]

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. In certain embodiments, the disclosure provides for a pharmaceutical composition comprising an immunomodulatory fusion protein with a pharmaceutically acceptable diluent, carrier, solubilizer, emulsifier, preservative and/or adjuvant.

In certain embodiments, the disclosure provides for a pharmaceutical composition comprising an immunomodulatory fusion protein with a pharmaceutically acceptable diluent, carrier, solubilizer, emulsifier, preservative and/or adjuvant.

In certain embodiments, the effective amount of a pharmaceutical composition comprising immunomodulatory fusion protein to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which the immunomodulatory fusion protein is being used, the route of administration, and the size (body weight, body surface or organ size) and/or condition (the age and general health) of the patient. In certain embodiments, the clinician can titer the dosage and modify the route of administration to obtain the optimal therapeutic effect.

VIII. Methods of Treating

The immunomodulatory fusion proteins and/or nucleic acids expressing them, described herein, are useful for treating a disorder associated with abnormal apoptosis or a differentiative process (e.g., cellular proliferative disorders (e.g., hyperproliferative disorders) or cellular differentiative disorders, such as cancer). Non-limiting examples of cancers that are amenable to treatment with the methods of the present disclosure are described below.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein, comprising, e.g., immunomodulatory fusion protein, can be administered to a patient who has cancer.

As used herein, the terms “cancer” (or “cancerous”), “hyperproliferative,” and “neoplastic” refer to cells having the capacity for autonomous growth (i.e., an abnormal state or condition characterized by rapidly proliferating cell growth). Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The immunomodulatory fusion proteins can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

In certain embodiments, the immunomodulatory fusion proteins disclosed herein are used to treat cancer. In certain embodiments, the immunomodulatory fusion proteins disclosed herein are used to treat melanoma, leukemia, lung cancer, breast cancer, prostate cancer, ovarian cancer, colon cancer, and brain cancer.

In certain embodiments, the immunomodulatory fusion proteins disclosed herein inhibit the growth and/or proliferation of tumor cells. In certain embodiments, the immunomodulatory fusion proteins disclosed herein reduce tumor size. In certain embodiments, the immunomodulatory fusion proteins disclosed herein inhibit metastases of a primary tumor.

In certain embodiments, administration of the immunomodulatory fusion proteins disclosed herein to a subject do not result in cytokine release syndrome after administration to a subject. In certain embodiments, the subject does not experience grade 4 cytokine release syndrome. In certain embodiments, the subject does not experience one or more symptoms associated with grade 4 cytokine release syndrome selected from the group consisting of hypotension, organ toxicity, fever and/or respiratory distress resulting in a need for supplemental Oxygen.

In certain embodiments, the administration of the fusion proteins disclosed herein, when administered either intravenously or intratumorally in a subject with cancer, the level of cytokines is increased in the serum of the subject after administration compared to IV or IT administration of recombinant IL-2 and/or IL-12. In certain embodiments, the cytokines that are increased in the serum of the subject are selected from INFγ, IP-10 and MCP-1.

### Combination Therapy

In some embodiments, the immunomodulatory fusion proteins are used in combination with other therapies. In some embodiments, the immunomodulatory fusion proteins are used in combination with additional therapeutic agents to treat cancer. For example, in some embodiments the immunomodulatory fusion proteins are used in combination with another immunotherapy. Exemplary immunotherapies include, but are not limited to, chimeric antigen receptor (CAR) T cell therapy, tumor-associated antigen targeting antibodies, immune checkpoint inhibitors, and cancer vaccines.

### I. Tumor-Associated Antigen Targeting Antibodies

In some aspects, the disclosure provides immunomodulatory fusion proteins to be used or performed in conjunction with antibodies that target tumor antigens.

Therapeutic monoclonal antibodies have been conceived as a class of pharmaceutically active agents which should allow tumor selective treatment by targeting tumor selective antigens or epitopes.

Methods of producing antibodies, and antigen binding fragments thereof, are well known in the art and are disclosed in, e.g., U.S. Pat. Nos. 7,247,301, 7,923,221, and U.S. Patent Application 2008/0138336, all of which are herein incorporated by reference in their entirety.

Therapeutic antibodies that can be used in the methods of the present disclosure include, but are not limited to, any of the art-recognized anti-cancer antibodies that are approved for use, in clinical trials, or in development for clinical use. In certain embodiments, more than one anticancer antibody can be included in the combination therapy of the present disclosure.

Non-limiting examples of anti-cancer antibodies include the following, without limitation: trastuzumab (HERCEPTIWM, by Genentech, South San Francisco, Calif), which is used to treat HER-2/neu positive breast cancer or metastatic breast cancer; bevacizumab (AVASTIWM by Genentech), which are used to treat colorectal cancer, metastatic colorectal cancer, breast cancer, metastatic breast cancer, non-small cell lung cancer, or renal cell carcinoma; rituximab (RITUXAWM by Genentech), which is used to treat non-Hodgkin's lymphoma or chronic lymphocytic leukemia; pertuzumab (OMNITARG™ by Genentech), which is used to treat breast cancer, prostate cancer, non-small cell lung cancer, or ovarian cancer; cetuximab (ERBITUX™ by ImClone Systems Incorporated, New York, N.Y.), which can be used to treat colorectal cancer, metastatic colorectal cancer, lung cancer, head and neck cancer, colon cancer, breast cancer, prostate cancer, gastric cancer, ovarian cancer, brain cancer, pancreatic cancer, esophageal cancer, renal cell cancer, prostate cancer, cervical cancer, or bladder cancer; IMC-1 Cl 1 (Im Clone Systems Incorporated), which is used to treat colorectal cancer, head and neck cancer, as well as other potential cancer targets; tositumomab and tositumomab and iodine I 131 (BEXXAR XM by Corixa Corporation, Seattle, Wash.), which is used to treat non-Hodgkin's lymphoma, which can be CD20 positive, follicular, non-Hodgkin's lymphoma, with and without transformation, whose disease is refractory to Rituximab and has relapsed following chemotherapy; In111 ibritumomab tiuxetan; Y90 ibritumomab tiuxetan; In111 ibritumomab tiuxetan and Y90 ibritumomab tiuxetan (ZEVALIN™ by Biogen Idee, Cambridge, Mass.), which is used to treat lymphoma or non-Hodgkin's lymphoma, which can include relapsed follicular lymphoma; relapsed or refractory, low grade or follicular non-Hodgkin's lymphoma; or transformed B-cell non-Hodgkin's lymphoma; EMD 7200 (EMD Pharmaceuticals, Durham, N.C.), which is used for treating non-small cell lung cancer or cervical cancer; SGN-30 (a genetically engineered monoclonal antibody targeted to CD30 antigen by Seattle Genetics, Bothell, Wash.), which is used for treating Hodgkin's lymphoma or non-Hodgkin's lymphoma; SGN-15 (a genetically engineered monoclonal antibody targeted to a Lewisy-related antigen that is conjugated to doxorubicin by Seattle Genetics), which is used for treating non-small cell lung cancer; SGN-33 (a humanized antibody targeted to CD33 antigen by Seattle Genetics), which is used for treating acute myeloid leukemia (AML) and myelodysplasia syndromes (MDS); SGN-40 (a humanized monoclonal antibody targeted to CD40 antigen by Seattle Genetics), which is used for treating multiple myeloma or non-Hodgkin's lymphoma; SGN-35 (a genetically engineered monoclonal antibody targeted to a CD30 antigen that is conjugated to auristatin E by Seattle Genetics), which is used for treating non-Hodgkin's lymphoma; SGN-70 (a humanized antibody targeted to CD70 antigen by Seattle Genetics), which is used for treating renal cancer and nasopharyngeal carcinoma; SGN-75 (a conjugate comprised of the SGN70 antibody and an Auristatin derivative by Seattle Genetics); and SGN-17/19 (a fusion protein containing antibody and enzyme conjugated to melphalan prodrug by Seattle Genetics), which is used for treating melanoma or metastatic melanoma.

### II. Immune Checkpoint Blockade

In some aspects, the disclosure provides immunomodulatory fusion proteins to be used or performed in conjunction with immune checkpoint inhibitors or immune checkpoint blockers.

T cell activation and effector functions are balanced by co-stimulatory and inhibitory signals, referred to as “immune checkpoints.” Inhibitory ligands and receptors that regulate T cell effector functions are overexpressed on tumorcells. Subsequently, agonists of co-stimulatory receptors or antagonists of inhibitory signals, result in the amplification of antigen-specific T cell responses. In contrast to therapeutic antibodies which target tumor cells directly, immune checkpoint blocker enhances endogenous anti-tumor activity.

In certain embodiments, the immune checkpoint blocker suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardall, D., Nature. 12: 252-264, 2012.

In certain embodiments, the immune checkpoint blocker is an antibody or an antigen-binding portion thereof, that disrupts or inhibits signaling from an inhibitory immunoregulator. In certain embodiments, the immune checkpoint blocker is a small molecule that disrupts or inhibits signaling from an inhibitory immunoregulator.

### EXAMPLES

The invention now being generally described, will be more readily understood by reference to the following examples, which are included merely for purposes of illustration of certain aspects and embodiments of the present invention, and is not intended to limit the invention.

### Example 1: Method of Preparation of Linear Constructs

The proteins of the present invention are typically made using recombinant DNA technology. In one exemplary embodiment, codon-optimized DNA sequences encoding comprising IL-12, IL-2, a collagen-binding protein, and albumin were synthesized and cloned into a pD2610-v1 vector. Plasmids were transformed into DH10B competent cells for expansion. Purified expression vectors were transiently transfected into HEK293 cells. Recombinant proteins were purified via anion exchange using Q Sepharose resin and preparative size exclusion chromatography (SEC). Concentrated protein was evaluated for product quality using analytical SEC. Proteins were subsequently polished with another round of preparative SEC prior to in vitro and in vivo evaluation

The proteins are isolated and purified using methods known in the art including centrifugation, depth filtration, cell lysis, homogenization, freeze thawing, affinity purification, gel filtration, ion exchange chromatography, hydrophobic interaction exchange chromatography, and mixed-mode chromatography.

### Example 2: Recombinant Collagen-Binding Fusion Proteins Bind Collagen In Vitro

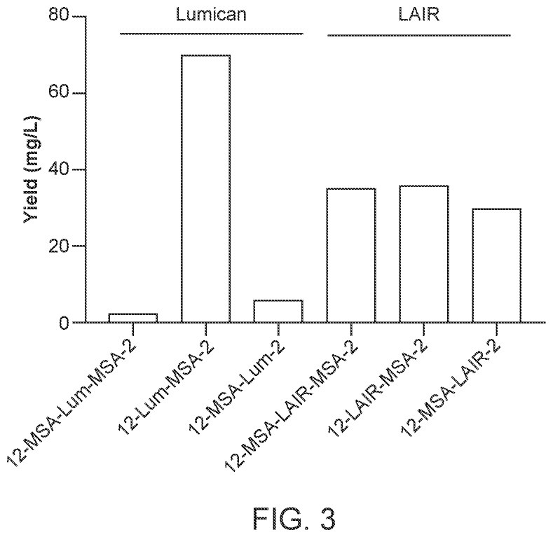

To evaluate the ability of collagen-binding immunomodulatory molecules to bind collagen, the collagen-binding fusion proteins expressed and purified as described in Example 1 were tested for their ability to bind to collagen I-coated plates by ELISA with linear fusion constructs and anti-His detection. Briefly, collagen I (Corning) coated 96-well plates were blocked at room temperature for 1 hour with 1% wt/vol bovine serum albumin (BSA). His×10-containing proteins were incubated on plates for 1.5 hours at increasing concentration. Wells were subsequently washed and incubated with an anti-His tag detection antibody (Abcam) for 1.5 hours. Bound His×10-tagged collagen-binding fusion proteins were visualized with TMB development followed by absorbance reads at 450 nm minus absorbance reads at 650 nm. As shown in FIG. 4A, LAIR-containing construct effected stronger binding to collagen compared to Lum-containing construct. Furthermore, placing Lumican between MSA and IL-2 enabled tighter binding to collagen than placing Lumican between MSA and IL-2. As shown in FIG. 4B, three LAIR-containing constructs using different spacer between LAIR and IL-2 effected comparable level of collagen binding

LAIR fusions potently bind collagen. LAIR fusion binds with tighter affinity than lumican fusion. Optionality to select weak or strong binding pending in vivo data and biological activity.

### Example 3: Recombinant Collagen-Binding Fusion Proteins Maintain IL-2 Cytokine Activity

To evaluate the ability of collagen-binding immunomodulatory molecules to maintain IL-2 cytokine activity in the presence of collage, samples were serially diluted in assay media and 50 μl diluted samples and 50 μl assay media added to either normal tissue-culture plates or collagen I (Corning) coated plates and incubated for 1 hour. About 25,000 CTLL-2 cells were subsequently transferred to each well in 100 μl assay media and incubated for 3 days. Following incubation, 20 μl Promega Substrate Cell Titer 96 Aqueous One Solution Reagent was added to each well, incubated at 37C, and absorbance read at 490 nm.

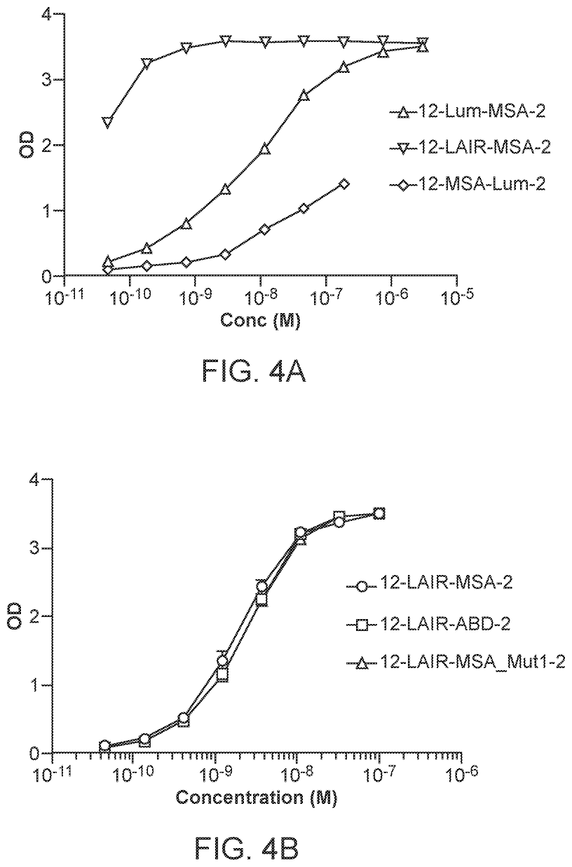

As shown in FIGS. 5A-5D, bi-functional constructs containing both IL-2 and IL-12 effected IL-2 activity at a level comparable to IL-2 alone. Furthermore, the IL-2 activity is not affected by collagen binding, and is independent of the choice of the spacer or the choice of the collagen binding domain.

### Example 4: Recombinant Collagen-Binding Fusion Proteins Maintain IL-12 Cytokine Activity

To evaluate the ability of collagen-binding immunomodulatory molecules to maintain IL-2 cytokine activity in the presence of collagen, samples were serially diluted in assay media and 50 μl diluted samples and 50 μl assay media were added to either normal tissue-culture plates or Corning collagen I-coated plates and incubated for 1 hr. About 15,000 2D6 cells were subsequently transferred to each well in 100 μl assay media and incubated for 4 days. Following incubation, 20 μl Promega Substrate Cell Titer 96 Aqueous One Solution Reagent was added to each well, incubated at 37° C., and absorbance read at 490 nm.

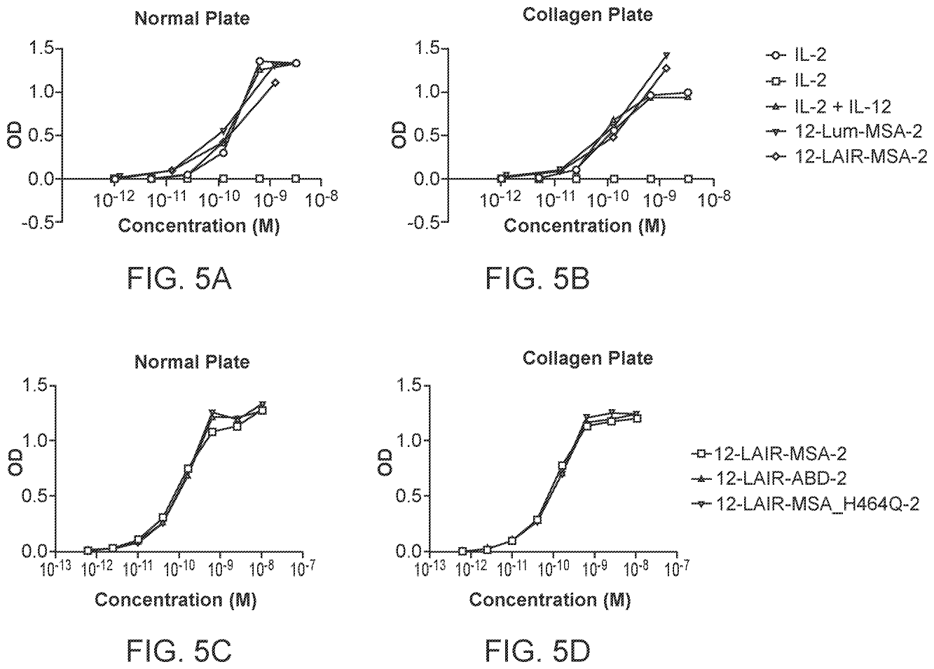

As shown in FIGS. 6A-6B, bi-functional constructs containing both IL-2 and IL-12 effected IL-12 activity at a level comparable to IL-12 alone. Furthermore, the IL-12 activity is not affected by collagen binding, and is independent of the choice of the collagen binding domain.

### Example 5: Synergistic Effect of Immunomodulatory Collagen-Binding Molecules and Anti-Tumor Antigen Antibody in Mouse Melanoma Tumor Model

To evaluate efficacy and toxicity of by bi-functional constructs and the combination of mono-functional constructs, C57BL/6 mice were inoculated on the right rear flank with 200,000 Bl6F10 cells in 0.1 ml PBS. After 9 days post-inoculation (day 0), mice were randomized into treatment groups (n=10). Mice were treated with intratumoral injections of 100 pmol on days 0 and 6 with 100 pmol of: (1) PBS, (2) a combination of an IL-2 mono-functional linear construct comprising an MSA (MSA-2) and an IL-12 mono-functional linear construct comprising an MSA (12-MSA), (3) a combination of an IL-2 mono-functional linear construct comprising an MSA and a collagen-binding domain (LAIR-MSA-2) and an IL-12 mono-functional linear construct comprising an MSA and collagen-binding domain (12-MSA-LAIR), (4) a bi-functional linear constructs comprising MSA and a collagen-binding domain 12-Lum-MSA-2, and (5) a bi-functional linear constructs comprising MSA and a collagen-binding domain 12-LAIR-MSA-2. Mice were monitored for tumor outgrowth and body weight loss at least twice a week and were euthanized if found to be moribund, if body weight loss >20%, or if tumor volume >3,000 mm3.

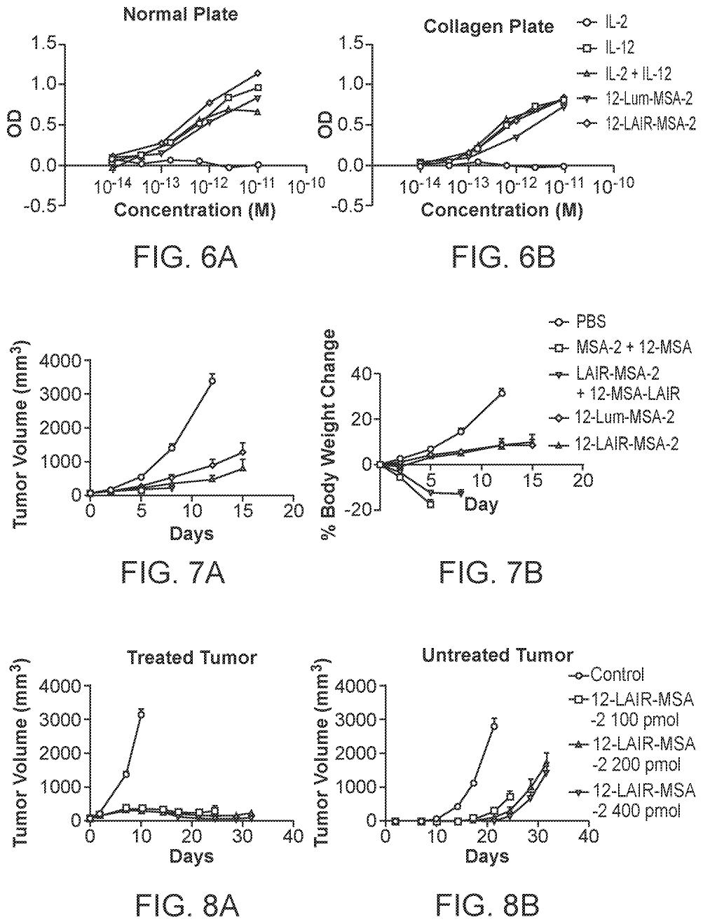

As shown in FIGS. 7A-7B, the tumor growth and body weight upon treatment by bi-functional constructs or combinations of mono-functional constructs show that both bi-functional linear constructs 12-Lum-MSA-2 and 12-LAIR-MSA-2 demonstrated superior safety profile indicated by lack of body weight loss, illustrating toxicity associated with systemic exposure of cytokines, compared to combination of mono-functional constructs regardless of whether the mono-functional constructs contain a collagen binding domain. Both 12-Lum-MSA-2 and 12-LAIR-MSA-2 effected significant tumor growth inhibition.

### Example 6: Linear Construct Monotherapy in B16F10 Model—Abscopal Effect and Dual Flank Model

To further evaluate the dose-response therapeutic efficacy of the bi-functional linear construct comprising MSA and a collagen-binding domain, 12-LAIR-MSA-2, was evaluated in a dual-flank inoculated subcutaneous Bl6F10 melanoma syngeneic model in C57BL/6 mice. Control C57BL/6 mice were inoculated with 200,000 Bl6F10 cells in 0.1 mL PBS on either the right rear flank (treated tumor plot) or on the left rear flank 10 days later (untreated tumor plot). Other mice on study were inoculated with 200,000 Bl6F10 cells in 0.1 mL PBS on the right rear flank and on the left rear flank 10d later. 8 days post-inoculation of the tumor on the right rear flank (day 0), mice were randomized into treatment groups (n=15). Mice were treated with intratumoral injections of indicated doses of 12-LAIR-MSA-2 on days 0, 6, and 12 in the right rear flank tumor. Mice were monitored for tumor outgrowth on both flanks and body weight loss at least twice a week and were euthanized if found to be moribund, if body weight loss >20%, or if total tumor volume >3,000 mm 3.

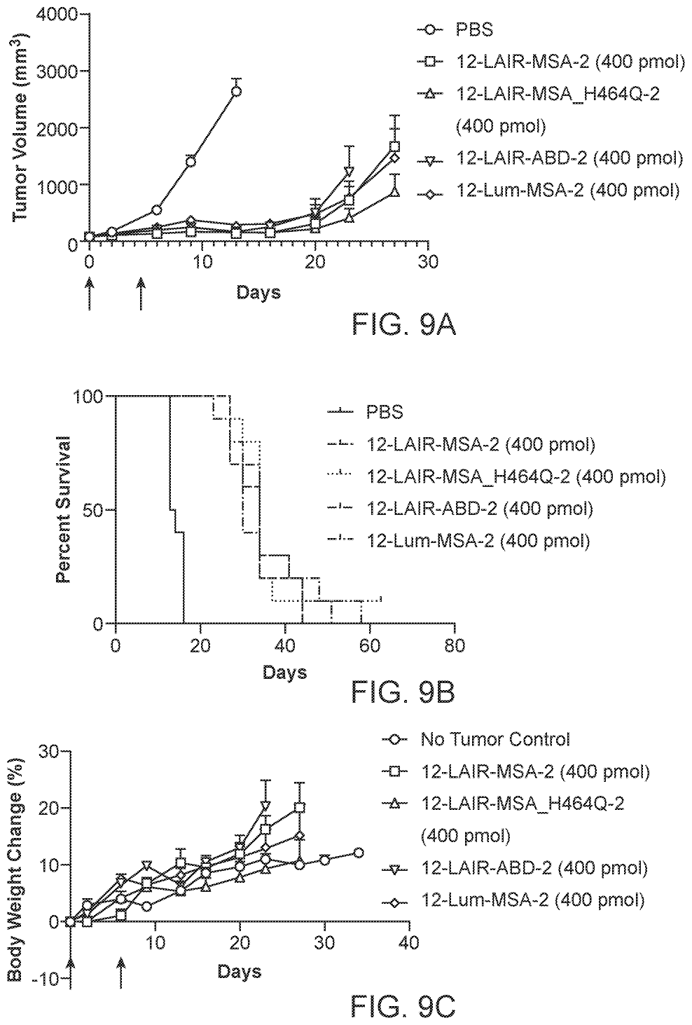

As shown in FIGS. 8A-8B, the bi-functional linear constructs 12-LAIR-MSA-2, at all dose levels tested, effected significantly tumor growth inhibition, both in the treated tumor (FIG. 8A) and the untreated tumor (FIG. 8B), demonstrating abscopal effect.

### Example 7: Linear Constructs Comparison in B16F10 Model

The efficacy and toxicity various bi-functional constructs were evaluated in a Bl6F10 mouse model. C57BL/6 mice were inoculated on the right rear flank with 200,000 Bl6F10 cells in 0.1 ml PBS. 7 days post-inoculation (day 0), mice were randomized into treatment groups (n=10). Mice were treated with intratumoral injections of 400 pmol of (1) PBS control, (2) 12-LAIR-MSA-2, (3) 12-LAIR-MSA H464Q-2, (4) 12-LAIR-ABD-2, and (5) 12-Lum-MSA-2 n days 0 and 6. Mice were monitored for tumor outgrowth and body weight loss at least twice a week and were euthanized if found to be moribund, if body weight loss >20%, or if tumor volume >3,000 mm 3.

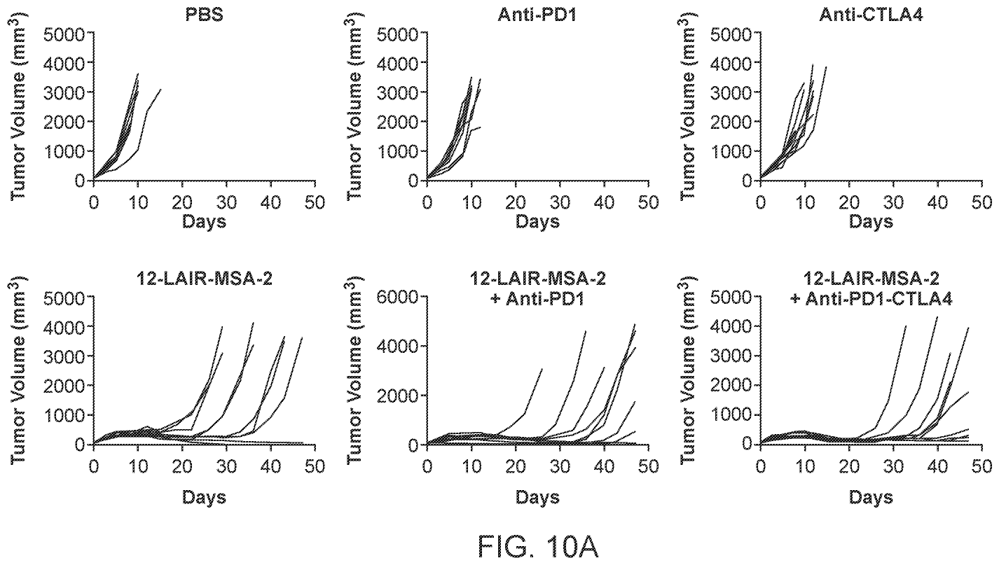

As shown in FIGS. 9A-9C, all bi-functional constructs tested effected significant tumor growth inhibition, demonstrated good safety profile reflected by the lack of body weight loss, and extended survival of the animals compared to PBS control group.

### Example 8: Linear Construct in B16F10 Model—Checkpoint Combination

To evaluate 12-LAIR-MSA-2 in combination with checkpoint inhibitors anti-PD1 or anti-CTLA, C57BL/6 mice were inoculated on the right rear flank with 200,000 Bl6F10 cells in 0.1 ml PBS. 7 days post-inoculation (day 0), mice were randomized into treatment groups (n=10). Mice were treated with intratumoral (IT) injections of PBS or 400 pmol of 12-LAIR-MSA-2 and intraperitoneal (IP) injections of isotype control (Rat IgG2a), anti-PD1 (clone RMP1-14), or anti-CTLA4 (9D9) as indicated. IT injections were performed on days 0, 6, and 12 while IP injections were performed BIW until end of study. Mice were monitored for tumor outgrowth and body weight loss at least twice a week and were euthanized if found to be moribund, if body weight loss >20%, or if tumor volume >3,000 mm 3.

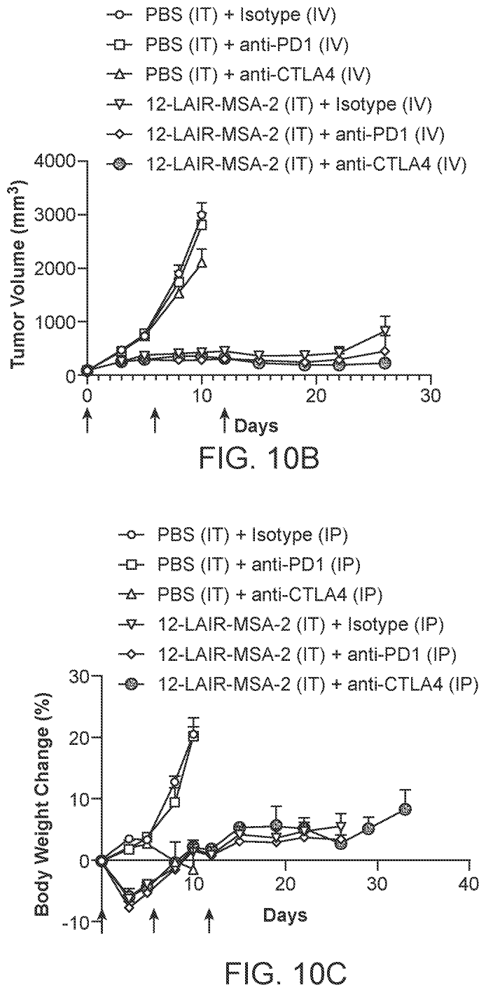

As shown in FIGS. 10A-10B, treatment with either anti-PD1 or anti-CTLA4 alone did not affect tumor growth inhibition. Treatment with bi-functional construct 12-LAIR-MSA-2 alone resulted in significant tumor growth inhibition. The anti-tumor activity of 12-LAIR-MSA-2 was further enhanced by the combination with either anti-PD1 or anti-CTLA4. As shown in FIG. 10C, the addition of either anti-PD1 or anti-CTLA4 to bi-functional construct 12-LAIR-MSA-2 did not result in additional weight loss compared to treatment with 12-LAIR-MSA-2 alone.

### Example 9: Linear Construct Monotherapy in MC38 Model—Safety and Efficacy

The dose-response therapeutic efficacy of the bi-functional linear construct comprising MSA and a collagen-binding domain, 12-LAIR-MSA-2, was evaluated in an MC38 model in C57BL/6 mice. C57BL/6 mice were inoculated on the right rear flank with 1,000,000 MC38 cells in 0.1 ml PBS. After 6 days post-inoculation (day 0), mice were randomized into treatment groups (n=10). Mice were treated with intratumoral injections of indicated doses of 12-LAIR-MSA-2 on days 0 and 6. Mice were monitored for tumor outgrowth and body weight loss at least twice a week and were euthanized if found to be moribund, if body weight loss >20%, or if tumor volume >3,000 mm 3.

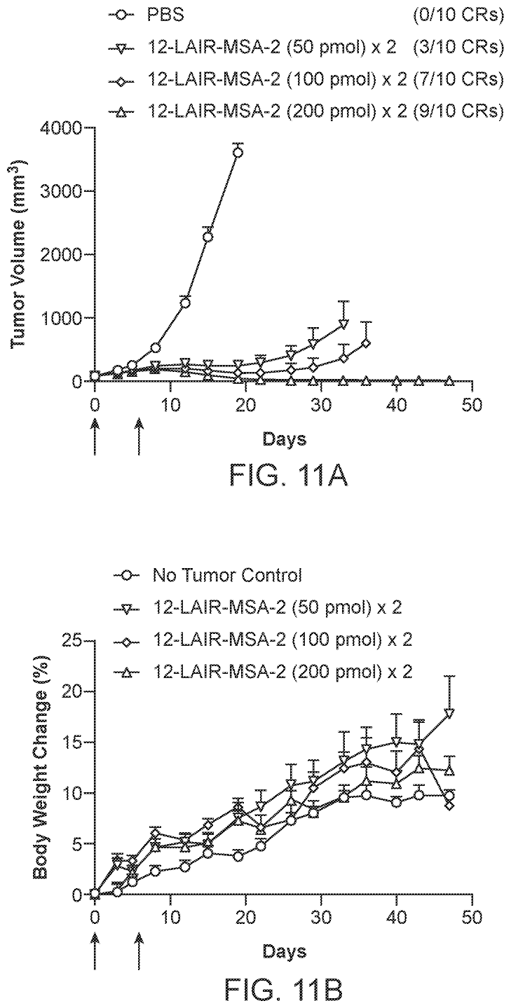

As shown in FIG. 11A, treatment with 12-LAIR-MSA-2 at all dose levels resulted in significant tumor growth inhibition. Furthermore, dose response was observed with treatment at the highest dose level resulting in the highest complete response (CR) rate. As shown in FIG. 11B, none of the treatment groups showed significant body weight loss.

### Example 10: Linear Constructs Comparison in MC38 Model

The efficacy and toxicity various bi-functional constructs were evaluated in a Bl6F10 mouse model. Mice were treated with intratumoral injections of indicated doses of PBS, 12-LAIR-MSA-2, 12-LAIR-ABD-2, and 12-Lum-MSA-2 on days 0 and 6. Mice were treated with intraperitoneal injections of isotype control (Rat IgG2a) or anti-PD1 (clone RMP1-14) BIW for three weeks if indicated. Mice were monitored for tumor outgrowth and body weight loss at least twice a week and were euthanized if found to be moribund, if body weight loss >20%, or if tumor volume >3,000 mm 3.

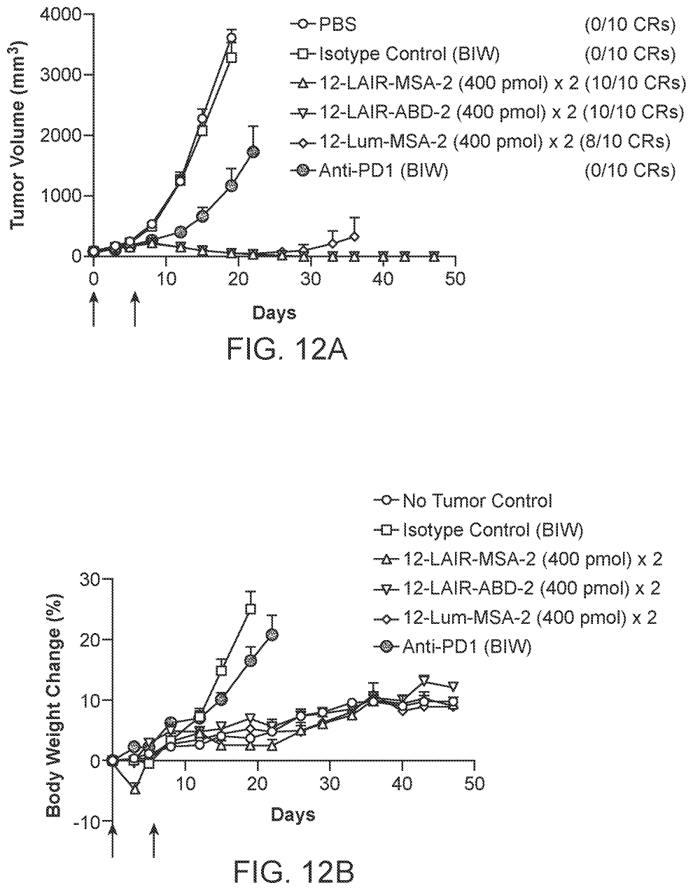

As shown in FIG. 12A, bi-functional constructs containing different collagen binding domains or spacer between IL-2 and the collagen binding domain, all resulted in significant tumor growth inhibition and CR rate. In comparison, treatment with anti-PD1 in the same model did not effect comparable degree of tumor growth control and did not result in any cures. As shown in FIG. 12B, none of the treatment groups showed significant body weight loss.

### Example 11: Linear Construct Monotherapy in CT26 Model—Safety and Efficacy

The dose-response therapeutic efficacy of the bi-functional linear construct comprising MSA and a collagen-binding domain, 12-LAIR-MSA-2, was evaluated in a CT26 model in BALB/c mice. BALB/c mice were inoculated on the right rear flank with 500,000 CT26 cells in 0.1 ml PBS. 6 days post-inoculation (day 0), mice were randomized into treatment groups (n=10). Mice were treated with intratumoral injections of indicated doses of PBS or 12-LAIR-MSA-2, with treatments administered on days 0, 6, and 12. Mice were treated with intraperitoneal injections of isotype control (Rat IgG2a) or anti-PD1 (clone RMP1-14) BIW for three weeks if indicated. Mice were monitored for tumor outgrowth and body weight loss at least twice a week and were euthanized if found to be moribund, if body weight loss >20%, or if tumor volume >3,000 mm 3.

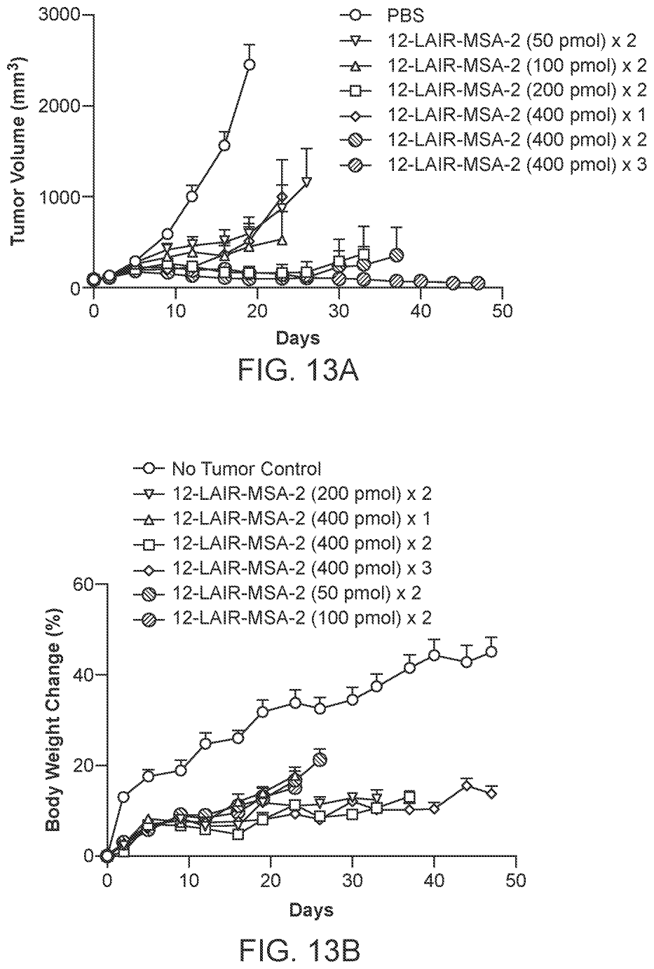

As shown in FIGS. 13A-13B tumor growth inhibition and body weight change upon treatment with 12-LAIR-MSA-2 at various dose levels or dose frequencies. None of the treatment groups showed body weight loss, and dose-dependent anti-tumor activity was observed.

### Example 12: Linear Constructs Comparison in MC38 Model

The efficacy and toxicity various bi-functional constructs were evaluated in a Bl6F10 mouse model. BALB/c mice were inoculated on the right rear flank with 500,000 CT26 cells in 0.1 ml PBS. 6 days post-inoculation (day 0), mice were randomized into treatment groups (n=10). Mice were treated with intratumoral injections of indicated doses of PBS, 12-LAIR-MSA-2, 12-LAIR-ABD-2, and 12-Lum-MSA-2 the indicated number of times, with treatments administered on days 0, 6, and 12. Mice were treated with intraperitoneal injections of isotype control (Rat IgG2a) or anti-PD1 (clone RMP1-14) BIW for three weeks if indicated. Mice were monitored for tumor outgrowth and body weight loss at least twice a week and were euthanized if found to be moribund, if body weight loss >20%, or if tumor volume >3,000 mm 3.

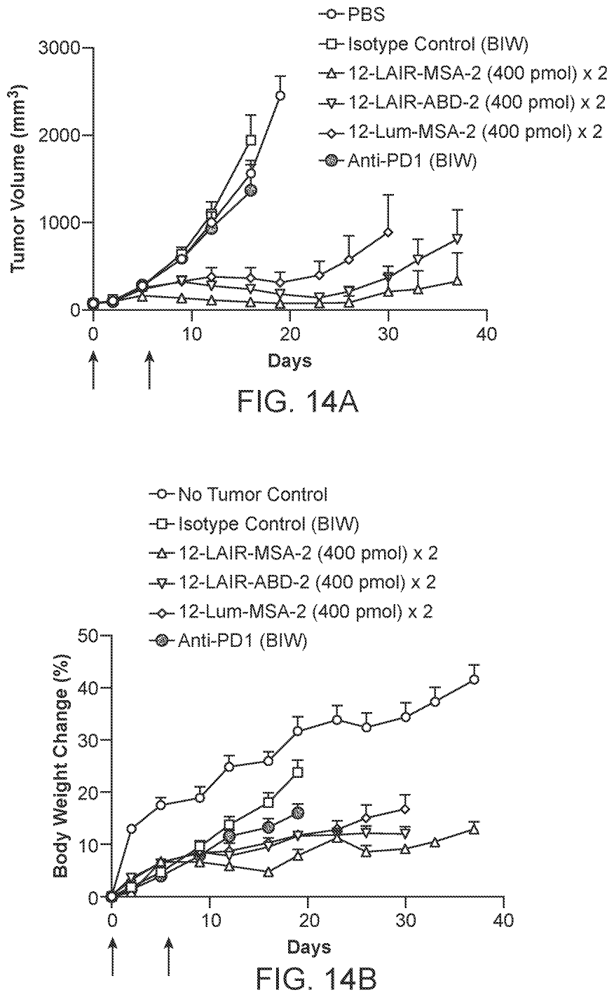

As shown in FIGS. 14A-14B, bi-functional constructs containing different collagen binding domains or spacer between IL-2 and the collagen binding domain, all resulted in significant tumor growth inhibition. In comparison, treatment with anti-PD1 in the same model did not effect tumor growth inhibition. None of the treatment groups showed body weight loss.

### Example 13: Linear Construct in B16F10 Model—IT and IV Administration

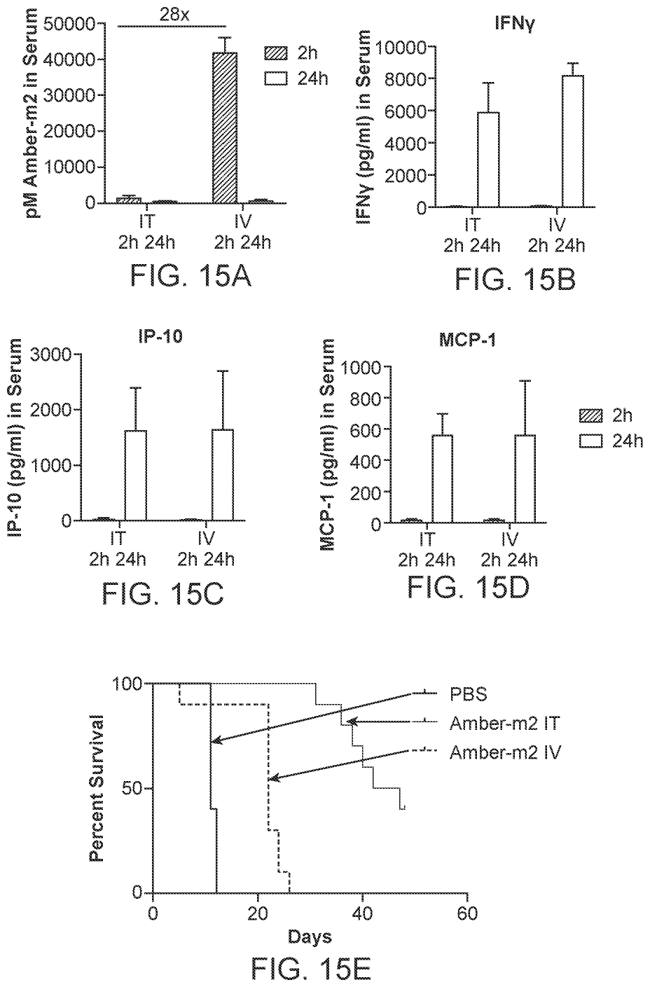

The efficacy of intratumoral (IT) compared to intravenous (IV) administration of the 12-LAIR-MSA-2 construct was evaluated in a Bl6F10 mouse model. C57BL/6 mice were inoculated on the right rear flank with 200,000 Bl6F10 cells in 0.1 ml PBS. Seven days post-inoculation (day 0), mice were randomized into treatment groups (n=10). Mice were treated with either intravenous or intratumoral injections of 400 pmol of PBS control or 12-LAIR-MSA-2. Two hours or 24 hours after administration, the amount of 12-LAIR-MSA-2 in the serum was measured (FIG. 15A). After two hours, there was a significant reduction in serum levels of the fusion protein when delivered IT compared to IV. After 24 hours, there were very low levels of fusion protein detected in mice administered the fusion protein either by IT or IV. Cytokines interferon gamma (INF-γ), interferon gamma inducible protein (IP-10) and monocyte chemoattractant protein-1 (MCP-1) were also measured either 2 h or 24 h after administration of the fusion protein by IT or IV administration (FIG. 15B-15D). The level of cytokines after 24 h was not significantly different when compared to mice that were administered the fusion protein by IT or IV. The efficacy of treatment, as measured by survival, however, was significantly improved in mice that were administered the fusion protein by IT administration as compared to IV administration (FIG. 15E). These results confirm that the fusion proteins described herein are effective at reducing serum concentrations of the fusion protein and improving survival of subjects when the fusion proteins are administered by intratumoral administration.

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

1. An immunomodulatory fusion protein comprising:
(i) an IL-2;
(ii) an IL-12;
(iii) a collagen-binding domain, wherein the collagen-binding domain comprises a leucine-rich repeat from a human proteoglycan Class II member of the small leucine-rich proteoglycan (SLRP) family; or
a human type I glycoprotein having an Ig-like domain, and
(iv) a linear polypeptide spacer.

2. The immunomodulatory fusion protein of claim 1, wherein the fusion protein is linear, a continuous chain, or a continuous polypeptide chain.

3-6. (canceled)

7. The immunomodulatory fusion protein of claim 1, wherein the IL-2 is at the N-terminus and the IL-12 is at the C-terminus.

8. The immunomodulatory fusion protein of claim 1, wherein the linear polypeptide spacer is positioned in between the IL-2 and the collagen-binding domain and/or the collagen-binding domain is positioned in between the IL-12 and the linear polypeptide spacer.

9-46. (canceled)

47. The immunomodulatory fusion protein of claim 1, further comprising a second linear polypeptide spacer.

48. The immunomodulatory fusion protein of claim 47, wherein the IL-12 is at the N terminus and is operably linked to the first linear polypeptide spacer, which is operably linked to the collagen binding domain, which is operably linked to the second linear polypeptide spacer, which is operably linked to the IL-2 at the C terminus of the protein, and wherein the protein is linear and/or the IL-12 is at the C terminus and is operably linked to the first linear polypeptide spacer, which is operably linked to the collagen binding domain, which is operably linked to the second linear polypeptide spacer, which is operably linked to the IL-2 at the N terminus of the protein, and wherein the protein is linear.

49-52. (canceled)

53. The immunomodulatory fusion protein of claim 1, wherein the leucine-rich repeat from a human proteoglycan Class II member of the small leucine-rich proteoglycan (SLRP) family comprises lumican.

54. The immunomodulatory fusion protein of claim 53, wherein the lumican comprises at least about 80% sequence identity to the amino acid sequence as set forth in SEQ ID NO: 11.

55. The immunomodulatory fusion protein of claim 1, wherein the human type I glycoprotein having an Ig-like domain is LAIR 1.

56. The immunomodulatory fusion protein of claim 55, wherein LAIR1 comprises at least about 80% sequence identity to the amino acid sequence as set forth in SEQ ID NO: 13 or SEQ ID NO: 14.

57. (canceled)

58. The immunomodulatory fusion protein of claim 1, wherein the human type I glycoprotein having an Ig-like domain is LAIR 2.

59. The immunomodulatory fusion protein of claim 58, wherein LAIR2 comprises at least 80% identity to the amino acid sequence as set forth in SEQ ID NO: 15.

60. The immunomodulatory fusion protein of claim 1, wherein the IL-2 comprises human IL-2 or human wild-type IL-2.

61. (canceled)

62. The immunomodulatory fusion protein of claim 1, wherein the IL-2 comprises at least about 80% sequence identity to the amino acid sequence as set forth in SEQ ID NO: 1 or SEQ ID NO: 2.

63. (canceled)

64. The immunomodulatory fusion protein of claim 1, wherein the IL-12 comprises human IL-12 or human wild-type IL-12.

65. (canceled)

66. The immunomodulatory fusion protein of claim 1, wherein the IL-12 comprises at least about 80% sequence identity to the amino acid sequence as set forth in SEQ ID NO: 5 or SEQ ID NO: 6.

67. The immunomodulatory fusion protein of claim 1, wherein the linear polypeptide spacer is an albumin or an albumin binding domain.

68. (canceled)

69. The immunomodulatory fusion protein of claim 67, wherein the albumin comprises human albumin or human serum albumin.

70. (canceled)

71. The immunomodulatory fusion protein of claim 67, wherein the albumin comprises at least about 80% sequence identity to the amino acid sequence as set forth in SEQ ID NOs: 16-18, or the albumin binding domain comprises at least about 80% sequence identity to the amino acid sequence as set forth in SEQ ID NO: 19.

72. (canceled)

73. The immunomodulatory fusion protein of claim 1, wherein the molecular weight of immunomodulatory fusion protein has a molecular weight of at least about 100-about 1000 kDa.

74. A pharmaceutical composition comprising an immunomodulatory fusion protein of claim 1, and a pharmaceutically acceptable carrier.

75-93. (canceled)

94. An immunomodulatory fusion protein comprising:
(i) an IL-2;
(ii) an IL-12;
(iii) a LAIR2 collagen-binding domain,
wherein LAIR2 comprises at least 80% identity to the amino acid sequence as set forth in SEQ ID NO: 15; and
(iv) an albumin;
wherein the albumin comprises at least about 80% sequence identity to the amino acid sequence as set forth in SEQ ID NO: 16-18.
